(19) United States
(12) Patent Application Publication
(10) Pub. No.: US 2012/0100619 A1
(43) Pub. Date: Apr. 26, 2012

(54) PICHIA PASTORIS LOCI ENCODING ENZYMES IN THE METHIONINE BIOSYNTHETIC PATHWAY

(76) Inventor: Juergen Nett, Grantham, NH (US)

(21) Appl. No.: 13/272,590

(22) Filed: Oct. 13, 2011

Related U.S. Application Data
(60) Provisional application No. 61/406,232, filed on Oct. 25, 2010.

(51) Int. Cl. C12N 15/81; C12N 15/54; C12N 1/19

(52) U.S. Cl. 435/483; 435/320.1; 435/254.23; 536/23.2

(57) ABSTRACT

Disclosed are the MET1, MET3, MET4, MET6, MET7, MET8, MET10, MET14, MET16, MET17, MET19, MET22, MET2, and MET28 genes encoding various enzymes in the methionine biosynthesis pathway of Pichia pastoris. The loci in the Pichia pastoris genome encoding these enzymes are useful sites for stable integration of heterologous nucleic acid molecules into the Pichia pastoris genome. The genes or gene fragments encoding the particular enzymes may be used as selection markers for constructing recombinant Pichia pastoris.

### CROSS REFERENCE TO RELATED APPLICATIONS

N/A

### BACKGROUND OF THE INVENTION

(1) Field of the Invention

The present invention relates to the isolation of the MET1, MET3, MET4, MET6, MET7, MET8, MET10, MET14, MET16, MET17, MET19, MET22, MET27 and MET28 genes encoding various enzymes in the methionine biosynthesis pathway of Pichia pastoris. The loci in the Pichia pastoris genome encoding these enzymes are useful sites for stable integration of heterologous nucleic acid molecules into the Pichia pastoris genome. The present invention further relates to genes or gene fragments encoding the particular enzymes, which may be used as selection markers for constructing recombinant Pichia pastoris.

(2) Description of Related Art

Recombinant bioengineering technology has enabled the ability to introduce heterologous or foreign genes into host cells that can then be used for the production and isolation of the proteins encoded by the heterologous genes. Numerous recombinant expression systems are available for expressing heterologous genes in mammalian cell culture, plant and insect cell culture, and microorganisms such as yeast and bacteria.

Yeast strains such as Pichia pastoris are well known in the art for production of heterologous recombinant proteins. DNA transformation systems in yeast have been developed (Cregg et al., Mol. Cell. Bio. 5: 3376 (1985)) in which an exogenous gene is integrated into the P. pastoris genome, often accompanied by a selectable marker gene which corresponds to an auxotrophy in the host strain for selection of the transformed cells. Biosynthetic marker genes include ADE1, ARG4, HIS4 and URA3 (Cereghino et al., Gene 263: 159-169 (2001)) as well as ARG1, ARG2, ARG3, HIS1, HIS2, HIS5 and HIS6 (U.S. Pat. No. 7,479,389) and URA5 (U.S. Pat. No. 7,514,253).

Extensive genetic engineering projects, such as the generation of a biosynthetic pathway not normally found in yeast, require the expression of several genes in parallel. In the past, very few loci within the yeast genome were known that enabled integration of an expression construct for protein production and thus only a small number of genes could be expressed. What is needed, therefore, is a method to express multiple proteins in Pichia pastoris using a myriad of available integration sites.

In order to extend the engineering of recombinant expression systems, and to further the development of novel expression systems such as the use of lower eukaryotic hosts to express mammalian proteins with human-like glycosylation, it is necessary to design improved methods and materials to extend the skilled artisan's ability to accomplish complex goals, such as integrating multiple genetic units into a host, with minimal disturbance of the genome of the host organism.

### BRIEF SUMMARY OF THE INVENTION

The present invention provides isolated polynucleotides comprising or consisting of nucleic acid sequences from the MET1, MET3, MET4, MET6, MET7, MET8, MET10, MET14, MET16, MET17, MET19, MET22, MET27, or MET28 locus of the yeast Pichia pastoris; including degenerate variants of these sequences; and related nucleic acid sequences and fragments. The invention also provides vectors and host cells comprising all or fragments of the isolated polynucleotides. The invention further provides host cells comprising a disruption, deletion, or mutation of a nucleic acid sequence from the MET1, MET3, MET4, MET6, MET7, MET8, MET10, MET14, MET16, MET17, MET19, MET22, MET27, or MET28 locus of Pichia pastoris wherein the host cells have reduced activity of the polypeptide encoded by the nucleic acid sequence compared to a host cell without the disruption, deletion, or mutation.

The present invention further provides methods and vectors for integrating heterologous DNA into the MET1, MET3, MET4, MET6, MET7, MET8, MET10, MET14, MET16, MET17, MET19, MET22, MET27, or MET28 locus of Pichia pastoris. The present invention further provides the use of a nucleic acid sequence encoding the enzyme encoded by any one of the loci for use as a selectable marker in methods in which a vector containing the nucleic acid sequence is transformed into the host cell that is auxotrophic for the enzyme.

In one aspect, the method provides a method for constructing recombinant Pichia pastoris that expresses one or more heterologous peptides, proteins, and/or functional nucleic acid molecules of interest in a Pichia pastoris host cell that is auxotrophic for methionine. The method comprises providing a methionine autotrophic strain of the Pichia pastoris that is met1, met3, met4, met6, met7, met8, met10, met14, met16, met17, met19, met22, met27, or met28 and transforming the auxotrophic strain with a vector, which comprises nucleic acid molecules encoding (i) a marker gene or open reading frame (ORF) that complements the auxotrophy of the auxotrophic strain operably linked to a promoter and (ii) a recombinant protein operably linked to a promoter, wherein the vector renders the auxotrophic strain prototrophic and the recombinant Pichia pastoris expresses one or more of the heterologous peptides, proteins, and/or functional nucleic acid molecules of interest.

In particular embodiments, the vector is an integration vector, which is capable of integrating into a particular location in the genome of the Pichia pastoris host cell in which case, the method comprises providing a methionine autotrophic strain of the Pichia pastoris that is met1, met3, met4, met6, met7, met8, met10, met14, met16, met17, met19, met22, met27, or met28 and transforming the auxotrophic strain with a integration vector, which comprises nucleic acid molecules encoding (i) a marker gene or open reading frame (ORF) that complements the auxotrophy of the auxotrophic strain operably linked to a promoter and (ii) one or more heterologous peptides, proteins, and/or functional nucleic acid molecules of interest operably linked to a promoter, wherein the integration vector is capable of targeting a particular region of the host cell genome and integrating into the targeted region of the host genome and the marker gene or ORF renders the auxotrophic strain prototrophic and the recombinant Pichia pastoris expresses the one or more heterologous peptides, proteins, and/or functional nucleic acid molecules of interest.

The met1, met3, met4, met6, met7, met8, met10, met14, met16, met17, met19, met22, met27, or met28 auxotrophic strain of the Pichia pastoris is constructed by transforming a Pichia pastoris host cell with a vector capable of integrating into the MET1, MET3, MET4, MET6, MET7, MET8, MET10, MET14, MET16, MET17, MET19, MET22, MET27, or MET28 locus wherein when the vector integrates into the locus to disrupt or delete the locus, the integration into the locus produces a recombinant Pichia pastoris that is auxotrophic for methionine.

In one aspect, the integration vector for constructing an auxotrophic strain comprises a heterologous nucleic acid fragment flanked on the 5′ end with a nucleic acid sequence from the 5′ region of the locus and on the 3′ end with a nucleic acid sequence from the 3′ region of the locus. The integration vector is capable of integrating into the genome by double-crossover homologous recombination. In particular aspects, the heterologous nucleic acid fragments encode one or more heterologous peptides, proteins, and/or functional nucleic acid molecules of interest.

In another aspect, the integration vector for constructing an auxotrophic strain comprises a nucleic acid fragment of the locus in which a region of the locus comprising the open reading frame (ORF) encoding Met1p, Met3p, Met4p, Met6p, Met7p, Met8p, Met10p, Met14p, Met16p, Met17p, Met19p, Met22p, Met27p, or Met28p has been excised. Thus, the integration vector comprises the 5′ region of the locus and the 3′ region of the locus and lacks part or all of the ORF encoding the Met1p, Met3p, Met4p, Met6p, Met7p, Met8p, Met10p, Met14p, Met16p, Met17p, Met19p, Met22p, Met27p, or Met28p. The integration vector is capable of integrating into the genome by double-crossover homologous recombination. In further aspects, the integration vector further includes one or more nucleic acid fragments, each encoding one or more heterologous peptides, proteins, and/or functional nucleic acid molecules of interest.

In a further aspect, provided is an integration vector comprising the open reading frame (ORF) encoding Met1p, Met3p, Met4p, Met6p, Met7p, Met8p, Met10p, Met14p, Met16p, Met17p, Met19p, Met22p, Met27p, or Met28p operably linked to a heterologous promoter and a heterologous transcription termination sequence. The integration vector can further include a nucleic acid molecule that targets a region of the host cell genome for integrating the integration vector thereinto that does not include the ORF and which can further include one or more nucleic acid molecules encoding one or more heterologous peptides, proteins, and/or functional nucleic acid molecules of interest. The integration vector comprising the ORF encoding the Met1p, Met3p, Met4p, Met6p, Met7p, Met8p, Met10p, Met14p, Met16p, Met17p, Met19p, Met22p, Met27p, or Met28p is useful for complementing the auxotrophy of a host cell auxotrophic for methionine as a result of a deletion or disruption of the MET1, MET3, MET4, MET6, MET7, MET8, MET10, MET14, MET16, MET17, MET19, MET22, MET27, or MET28 locus, respectively.

In another aspect, provided is an integration vector comprising the open reading frame encoding Met1p, Met3p, Met4p, Met6p, Met7p, Met8p, Met10p, Met14p, Met16p, Met17p, Met19p, Met22p, Met27p, or Met28p and the flanking promoter sequence and transcription termination sequence. The integration vector can further include a nucleic acid molecule that targets a region of the host cell genome for integrating the integration vector thereinto that does not include the ORF and which can further include one or more nucleic acid molecules encoding one or more heterologous peptides, proteins, and/or functional nucleic acid molecules of interest. The integration vector comprising the ORF encoding the Met1p, Met3p, Met4p, Met6p, Met7p, Met8p, Met10p, Met14p, Met16p, Met17p, Met19p, Met22p, Met27p, or Met28p is useful for complementing the auxotrophy of a host cell auxotrophic for methionine as a result of a deletion or disruption of the MET1, MET3, MET4, MET6, MET7, MET8, MET10, MET14, MET16, MET17, MET19, MET22, MET27, or MET28 locus, respectively.

In further aspects, provided is an expression system comprising (a) a Pichia pastoris host cell in which all or part of the endogenous MET1, MET3, MET4, MET6, MET7, MET8, MET10, MET14, MET16, MET17, MET19, MET22, MET27, or MET28 locus has been deleted or disrupted to render the host cell auxotrophic for methionine; and (b) an integration vector comprising (1) a nucleic acid molecule encoding a gene or open reading frame that complements the auxotrophy; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In further aspects, provided is an expression system comprising (a) a Pichia pastoris host cell in which all or part of the endogenous MET1, MET3, MET4, MET6, MET7, MET8, MET10, MET14, MET16, MET17, MET19, MET22, MET27, or MET28 gene has been deleted or disrupted to render the host cell auxotrophic for methionine; and (b) an integration vector comprising (1) a nucleic acid molecule encoding a gene or open reading frame that complements the auxotrophy; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In further aspects, provided is an expression system comprising (a) a Pichia pastoris host cell in which all or part of the endogenous gene encoding Met1p, Met3p, Met4p, Met6p, Met7p, Met8p, Met10p, Met14p, Met16p, Met17p, Met19p, Met22p, Met27p, or Met28p, respectively, has been deleted or disrupted to render the host auxotrophic for methionine; and (b) an integration vector comprising (1) a nucleic acid molecule encoding a gene or open reading frame that complements the auxotrophy; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In further aspects, provided is an expression system comprising (a) a Pichia pastoris host cell in which all or part of the endogenous MET1, MET3, MET4, MET6, MET7, MET8, MET10, MET14, MET16, MET17, MET19, MET22, MET27, or MET28 gene or locus has been deleted or disrupted to render the host cell auxotrophic for methionine; and (b) an integration vector comprising (1) a nucleic acid molecule encoding a gene or open reading frame that complements the auxotrophy; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In further aspects, provided is an expression system comprising (a) a Pichia pastoris host cell in which all or part of the endogenous gene encoding Met1p, Met3p, Met4p, Met6p, Met7p, Met8p, Met10p, Met14p, Met16p, Met17p, Met19p, Met22p, Met27p, or Met28p, respectively, has been deleted or disrupted to render the host cell auxotrophic for methionine; and (b) an integration vector comprising (1) a nucleic acid molecule encoding a gene or open reading frame that complements the auxotrophy; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In further aspects, provided is an expression system comprising (a) a Pichia pastoris host cell in which all or part of the endogenous MET1, MET3, MET4, MET6, MET7, MET8, MET10, MET14, MET16, MET17, MET19, MET22, MET27, or MET28 gene encoding Met1p, Met3p, Met4p, Met6p, Met7p, Met8p, Met10p, Met14p, Met16p, Met17p, Met19p, Met22p, Met27p, or Met28p, respectively, has been deleted or disrupted to render the host cell auxotrophic for methionine; and (b) an integration vector comprising (1) a nucleic acid molecule encoding a gene or open reading frame that complements the auxotrophy; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In further aspects, provided is an expression system comprising (a) a Pichia pastoris host cell in which all or part of the endogenous MET1, MET3, MET4, MET6, MET7, MET8, MET10, MET14, MET16, MET17, MET19, MET22, MET27, or MET28 gene or locus has been deleted or disrupted to render the host cell auxotrophic for methionine; and (b) an integration vector comprising (1) a nucleic acid molecule encoding the Met1p, Met3p, Met4p, Met6p, Met7p, Met8p, Met10p, Met14p, Met16p, Met17p, Met19p, Met22p, Met27p, or Met28p, respectively; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In further aspects, provided is an expression system comprising (a) a Pichia pastoris host cell in which all or part of the endogenous MET1, MET3, MET4, MET6, MET7, MET8, MET10, MET14, MET16, MET17, MET19, MET22, MET27, or MET28 gene or locus encoding Met1p, Met3p, Met4p, Met6p, Met7p, Met8p, Met10p, Met14p, Met16p, Met17p, Met19p, Met22p, Met27p, or Met28p, respectively, has been deleted or disrupted to render the host cell auxotrophic for methionine; and (b) an integration vector comprising (1) a nucleic acid molecule encoding the Met1p, Met3p, Met4p, Met6p, Met7p, Met8p, Met10p, Met14p, Met16p, Met17p, Met19p, Met22p, Met27p, or Met28p, respectively; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

Also, provided is a method for producing a recombinant Pichia pastoris host cell that expresses one or more heterologous peptides, proteins, and/or functional nucleic acid molecules of interest peptide comprising (a) providing the host cell in which all or part of the endogenous MET1, MET3, MET4, MET6, MET7, MET8, MET10, MET14, MET16, MET17, MET19, MET22, MET27, or MET28 gene encoding Met1p, Met3p, Met4p, Met6p, Met7p, Met8p, Met10p, Met14p, Met16p, Met17p, Met19p, Met22p, Met27p, or Met28p, respectively, has been deleted or disrupted to render the host cell auxotrophic for methionine; and (a) transforming the host cell with an integration vector comprising (1) a nucleic acid molecule encoding a gene or open reading frame that complements the auxotrophy; (2) a nucleic acid molecule having one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination, wherein the transformed host cell produces the one or more heterologous peptides, proteins, and/or functional nucleic acid molecules of interest.

Also, provided is a method for producing a recombinant Pichia pastoris host cell that expresses one or more heterologous peptides, proteins, and/or functional nucleic acid molecules of interest ptide comprising (a) providing the host cell in which all or part of the endogenous MET1, MET3, MET4, MET6, MET7, MET8, MET10, MET14, MET16, MET17, MET19, MET22, MET27, or MET28 gene encoding Met1p, Met3p, Met4p, Met6p, Met7p, Met8p, Met10p, Met14p, Met16p, Met17p, Met19p, Met22p, Met27p, or Met28p, respectively, has been deleted or disrupted to render the host cell auxotrophic for methionine; and (a) transforming the host cell with an integration vector comprising (1) a nucleic acid molecule encoding the Met1p, Met3p, Met4p, Met6p, Met7p, Met8p, Met10p, Met14p, Met16p, Met17p, Met19p, Met22p, Met27p, or Met28p, respectively; (2) a nucleic acid molecule having one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination, wherein the transformed host cell produces the one or more heterologous peptides, proteins, and/or functional nucleic acid molecules of interest.

Further provided is an isolated nucleic acid molecule comprising the MET1, MET3, MET4, MET6, MET7, MET8, MET10, MET14, MET16, MET17, MET19, MET22, MET27, or MET28 gene of Pichia pastoris.

International Application No. WO2009085135 discloses that operably linking an auxotrophic marker gene or ORF to a minimal promoter in the integration vector, that is a promoter that has low transcriptional activity, enabled the production of recombinant host cells that contain a sufficient number of copies of the integration vector integrated into the genome of the auxotrophic host cell to render the cell prototrophic and which render the cells capable of producing amounts of the recombinant protein or functional nucleic acid molecule of interest that are greater than the amounts that would be produced in a cell that contained only one copy of the integration vector integrated into the genome.

Therefore, provided is a method in which a methionine autotrophic strain of the Pichia pastoris that is met1, met3, met4, met6, met7, met8, met10, met14, met16, met17, met19, met22, met27, or met28 is obtained or constructed and an integration vector is provided that is capable of integrating into the genome of the auxotrophic strain and which comprises nucleic acid molecules encoding a marker gene or ORF that compliments the auxotrophy and is operably linked to a weak promoter, an attenuated endogenous or heterologous promoter, a cryptic promoter, or a truncated endogenous or heterologous promoter and a recombinant protein. Host cells in which a number of the integration vectors have been integrated into the genome to compliment the auxotrophy of the host cell are selected in medium that lacks the metabolite that compliments the auxotrophy and maintained by propagating the host cells in medium that lacks the metabolite that compliments the auxotrophy or in medium that contains the metabolite because in that case, cells that evict the vectors including the marker will grow more slowly.

In a further embodiment, provided is an expression system comprising (a) a host cell in which all or part of the endogenous MET1, MET3, MET4, MET6, MET7, MET8, MET10, MET14, MET16, MET17, MET19, MET22, MET27, or MET28 gene or locus has been deleted or disrupted to render the host cell auxotrophic for methionine; and (b) an integration vector comprising (1) a nucleic acid molecule comprising an open reading frame (ORF) encoding a function that is complementary to the function of the endogenous gene encoding the auxotrophic selectable marker protein and which is operably linked to a weak promoter, an attenuated endogenous or heterologous promoter, a cryptic promoter, a truncated endogenous or heterologous promoter, or no promoter; (2) a nucleic acid molecule having an insertion site for the insertion of one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination.

In a further still embodiment, provided is a method for expression of a recombinant protein in a host cell comprising (a) providing the host cell in which all or part of the endogenous MET1, MET3, MET4, MET6, MET7, MET8, MET10, MET14, MET16, MET17, MET19, MET22, MET27, or MET28 gene or locus has been deleted or disrupted to render the host cell auxotrophic for methionine; and (a) transforming the host cell with an integration vector comprising (1) a nucleic acid molecule comprising an open reading frame (ORF) encoding a function that is complementary to the function of the endogenous gene encoding the auxotrophic selectable marker protein and which is operably linked to a weak promoter, an attenuated endogenous or heterologous promoter, a cryptic promoter, a truncated endogenous or heterologous promoter, or no promoter; (2) a nucleic acid molecule having one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination, wherein the transformed host cell produces the recombinant protein.

In a further still embodiment, provided is a method for expression of a recombinant protein in a host cell comprising (a) providing the host cell in which all or part of the endogenous gene encoding Met1p, Met3p, Met4p, Met6p, Met7p, Met8p, Met10p, Met14p, Met16p, Met17p, Met19p, Met22p, Met27p, or Met28p, has been deleted or disrupted to render the host cell auxotrophic for methionine; and (a) transforming the host cell with an integration vector comprising (1) a nucleic acid molecule comprising an open reading frame (ORF) encoding a function that is complementary to the function of the endogenous gene encoding the auxotrophic selectable marker protein and which is operably linked to a weak promoter, an attenuated endogenous or heterologous promoter, a cryptic promoter, a truncated endogenous or heterologous promoter, or no promoter; (2) a nucleic acid molecule having one or more expression cassettes comprising a nucleic acid molecule encoding one or more heterologous peptides, proteins, and/or functional nucleic acid molecules of interest, and (3) a targeting nucleic acid molecule that directs insertion of the integration vector into a particular location of the genome of the host cell by homologous recombination, wherein the transformed host cell produces the recombinant protein.

In further still aspects, the integration vector comprises multiple insertion sites for the insertion of one or more expression cassettes encoding the one or more heterologous peptides, proteins and/or functional nucleic acid molecules of interest. In further still aspects, the integration vector comprises more than one expression cassette. In further still aspects, the integration vector comprises little or no homologous DNA sequence between the expression cassettes. In further still aspects, the integration vector comprises a first expression cassette encoding a light chain of a monoclonal antibody and a second expression cassette encoding a heavy chain of a monoclonal antibody.

Further provided is a plasmid vector that is capable of integrating into a Pichia pastoris locus selected from the group consisting of MET1, MET3, MET4, MET6, MET7, MET8, MET10, MET14, MET16, MET17, MET19, MET22, MET27, or MET28. In further aspects, the plasmid vector of claim 1 comprising a nucleotide sequence with at least 95% identity to a nucleotide sequence comprising at least 25, 50, 75, 100, 125, 150, 175, or 200 contiguous nucleotides of SEQ ID NO:1, 3, 5, 7, 9, 11, 13, 15, 17, 19, 21, 23, 25, or 27. The plasmid vector can in further aspects include a nucleic acid molecule encoding a heterologous peptide, protein, or functional nucleic acid molecule of interest.

Further provided is a method for producing a recombinant Pichia pastoris auxotrophic for methionine, comprising: transforming a Pichia pastoris host cell with the plasmid vector capable of integrating into the MET1, MET3, MET4, MET6, MET7, MET8, MET10, MET14, MET16, MET17, MET19, MET22, MET27, or MET28 locus, wherein the plasmid vector integrates into the locus to disrupt or delete the locus to produce the recombinant Pichia pastoris auxotrophic for methionine.

Further provided is a recombinant Pichia pastoris produced by any one of the above-mentioned methods.

Further provided is a nucleic acid molecule comprising a nucleotide sequence with at least 95% to a nucleotide sequence comprising at least 25, 50, 75, 100, 125, 150, 175, or 200 contiguous nucleotides of SEQ ID NO:1, 3, 5, 7, 9, 11, 13, 15, 17, 19, 21, 23, 25, or 27.

Further provided is a plasmid vector comprising a nucleic acid sequence encoding a Pichia pastoris enzyme selected from the group consisting of Met1p, Met3p, Met4p, Met6p, Met7p, Met8p, Met10p, Met14p, Met16p, Met17p, Met19p, Met22p, Met27p, or Met28p. In particular aspects, the plasmid vector comprises a nucleotide sequence with at least 95% identity to a nucleotide sequence comprising at least 25, 50, 75, 100, 125, 150, 175, or 200 contiguous nucleotides of SEQ ID NO:1, 3, 5, 7, 9, 11, 13, 15, 17, 19, 21, 23, 25, or 27.

Further provided is a method for rendering a recombinant Pichia pastoris that is auxotrophic for methionine into a recombinant Pichia pastoris prototrophic for methionine comprising: (a) providing a recombinant met1, met3, met4, met6, met7, met8, met10, met14, met16, met17, met19, met22, met27, or met28 Pichia pastoris host cell auxotrophic for methionine; and (b) transforming the recombinant Pichia pastoris with a plasmid vector encoding the enzyme that complements the auxotrophy to render the recombinant Pichia pastoris auxotrophic for methionine into a Pichia pastoris prototrophic for methionine.

In particular aspects, the host cell auxotrophic for methionine has a deletion or disruption of the MET1, MET3, MET4, MET6, MET7, MET8, MET10, MET14, MET16, MET17, MET19, MET22, MET27, or MET28 locus.

In further aspects, the plasmid vector encoding the enzyme that complements the auxotrophy integrates into a location in the genome of the host cell. In further aspects, the location is any location within the genome but is not the MET1, MET3, MET4, MET6, MET7, MET8, MET10, MET14, MET16, MET17, MET19, MET22, MET27, or MET28 locus, for example, for example, the plasmid vector integrates in a location of the genome for ectopic expression of the nucleic acid molecule encoding the MET1, MET3, MET4, MET6, MET7, MET8, MET10, MET14, MET16, MET17, MET19, MET22, MET27, or MET28 gene or open reading frame encoding the Met1p, Met3p, Met4p, Met6p, Met7p, Met8p, Met10p, Met14p, Met16p, Met17p, Met19p, Met22p, Met27p, or Met28p and which complements the auxotrophy.

In further still aspects, the Pichia pastoris host cell that has been modified to be capable of producing glycoproteins having hybrid or complex N-glycans.

In a further aspect, provided are host cells in which at least one of Met1p, Met3p, Met4p, Met6p, Met7p, Met8p, Met10p, Met14p, Met16p, Met17p, Met19p, Met22p, Met27p, or Met28p is ectopically expressed in the host cell. In further aspects, the host cell has one or more of the MET1, MET3, MET4, MET6, MET7, MET8, MET10, MET14, MET16, MET17, MET19, MET22, MET27, or MET28 loci deleted or disrupted and the host cell ectopically expresses the Met1p, Met3p, Met4p, Met6p, Met7p, Met8p, Met10p, Met14p, Met16p, Met17p, Met19p, Met22p, Met27p, or Met28p encoded by the deleted or disrupted loci. Further provided is a host cell that is prototrophic for methionine but wherein one or more of Met1p, Met3p, Met4p, Met6p, Met7p, Met8p, Met10p, Met14p, Met16p, Met17p, Met19p, Met22p, Met27p, or Met28p is ectopically expressed.

Further provided are isolated nucleic aid molecules comprising the 5′ or 3′ non-coding region of the MET1, MET3, MET4, MET6, MET7, MET8, MET10, MET14, MET16, MET17, MET19, MET22, MET27, or MET28 locus. Further provided are expression vectors comprising a nucleic acid molecule encoding a sequence of interest operably linked at the 5′ end with the 5′ non-coding region of the MET1, MET3, MET4, MET6, MET7, MET8, MET10, MET14, MET16, MET17, MET19, MET22, MET27, or MET28 locus. Further provided are expression vectors comprising a nucleic acid molecule encoding a sequence of interest operably linked at the 3′ end with the 3′ non-coding region of the MET1, MET3, MET4, MET6, MET7, MET8, MET10, MET14, MET16, MET17, MET19, MET22, MET27, or MET28 locus. Further provided are expression vectors comprising a nucleic acid molecule encoding a sequence of interest operably linked at the 5′ end with the 5′ non-coding region of the MET1, MET3, MET4, MET6, MET7, MET8, MET10, MET14, MET16, MET17, MET19, MET22, MET27, or MET28 locus and at the 3′ end with the 3′ non-coding region of the MET1, MET3, MET4, MET6, MET7, MET8, MET10, MET14, MET16, MET17, MET19, MET22, MET27, or MET28 locus.

Further provided are polyclonal and monoclonal antibodies against Met1p, Met3p, Met4p, Met6p, Met7p, Met8p, Met10p, Met14p, Met16p, Met17p, Met19p, Met22p, Met27p, or Met28p.

### DEFINITIONS

Unless otherwise defined herein, scientific and technical terms and phrases used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

The genetic nomenclature for naming chromosomal genes of yeast is used herein. Each gene, allele, or locus is designated by three italicized letters. Dominant alleles are denoted by using uppercase letters for all letters of the gene symbol, for example, MET8 for the methionine 8 gene, whereas lowercase letters denote the recessive allele, for example, the auxotrophic marker for methionine 8, met8. Wild-type genes are denoted by superscript “+” and mutants by a “−” superscript. The symbol Δ can denote partial or complete deletion. Insertion of genes follow the bacterial nomenclature by using the symbol “::”, for example, trp2::MET8 denotes the insertion of the MET8 gene at the TRP2 locus, in which MET8 is dominant (and functional) and trp2 is recessive (and defective). Proteins encoded by a gene are referred to by the relevant gene symbol, non-italicized, with an initial uppercase letter and usually with the suffix ‘p”, for example, the methionine 8 protein encoded by MET8 is Met8p. Phenotypes are designated by a non-italic, three letter abbreviation corresponding to the gene symbol, initial letter in uppercase. Wild-type strains are indicated by a “+” superscript and mutants are designated by a “−” superscript. For example, Met8⁺ is a wild-type phenotype whereas met8⁻ is an auxotrophic phenotype (requires methionine).

The term “vector” as used herein is intended to refer to a nucleic acid molecule capable of transporting another nucleic acid molecule to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”).

The term “integration vector” refers to a vector that can integrate into a host cell and which carries a selection marker gene or open reading frame (ORF), a targeting nucleic acid molecule, one or more genes or nucleic acid molecules of interest, and a nucleic acid sequence that functions as a microorganism autonomous DNA replication start site, herein after referred to as an origin of DNA replication, such as ORI for bacteria. The integration vector can only be replicated in the host cell if it has been integrated into the host cell genome by a process of DNA recombination such as homologous recombination that integrates a linear piece of DNA into a specific locus of the host cell genome. For example, the targeting nucleic acid molecule targets the integration vector to the corresponding region in the genome where it then by homologous recombination integrates into the genome.

The term “selectable marker gene”, “selection marker gene”, “selectable marker sequence” or the like refers to a gene or nucleic acid sequence carried on a vector that confers to a transformed host a genetic advantage with respect to a host that does not contain the marker gene. For example, the P. pastoris URA5 gene is a selectable marker gene because its presence can be selected for by the ability of cells containing the gene to grow in the absence of uracil. Its presence can also be selected against by the inability of cells containing the gene to grow in the presence of 5-FOA. Selectable marker genes or sequences do not necessarily need to display both positive and negative selectability. Non-limiting examples of marker sequences or genes from P. pastoris include ADE1, ADE2 ARG4, HIS4, LYS2, URA5, and URA3. In general, a selectable marker gene as used the expression systems disclosed herein encodes a gene product that complements an auxotrophic mutation in the host. An auxotrophic mutation or auxotrophy is the inability of an organism to synthesize a particular organic compound or metabolite required for its growth (as defined by IUPAC). An auxotroph is an organism that displays this characteristic; auxotrophic is the corresponding adjective. Auxotrophy is the opposite of prototrophy.

The term “a targeting nucleic acid molecule” refers to a nucleic acid molecule carried on the vector plasmid that directs the insertion by homologous recombination of the vector integration plasmid into a specific homologous locus in the host called the “target locus”.

The term “sequence of interest” or “gene of interest” or “nucleic acid molecule of Interest” refers to a nucleic acid sequence, typically encoding a protein or a functional RNA, that is not normally produced in the host cell. The methods disclosed herein allow efficient expression of one or more sequences of interest or genes of interest stably integrated into a host cell genome. Non-limiting examples of sequences of interest include sequences encoding one or more polypeptides having an enzymatic activity, e.g., an enzyme which affects N-glycan synthesis in a host such as mannosyltransferases, N-acetylglucosaminyltransferases, UDP-N-acetylglucosamine transporters, galactosyltransferases, UDP-N-acetylgalactosyltransferase, sialyltransferases, fucosyltransferases, erythropoietin, cytokines such as interferon-α, interferon-β, interferon-γ, interferon-ω, and granulocyte-CSF, coagulation factors such as factor VIII, factor IX, and human protein C, soluble IgE receptor α-chain, IgG, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, and osteoprotegerin.

The term “operatively linked” refers to a linkage in which a expression control sequence is contiguous with the gene or sequence of interest or selectable marker gene or sequence to control expression of the gene or sequence, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell,” “expression host system,” “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “eukaryotic” refers to a nucleated cell or organism, and includes insect cells, plant cells, mammalian cells, animal cells, and lower eukaryotic cells.

The term “lower eukaryotic cells” includes yeast, unicellular and multicellular or filamentous fungi. Yeast and fungi include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa.

The term “peptide” as used herein refers to a short polypeptide, e.g., one that is typically less than about 50 amino acids long and more typically less than about 30 amino acids long. The term as used herein encompasses analogs, derivatives, and mimetics that mimic structural and thus, biological function of polypeptides and proteins.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, 20, and 25 amino acids in length. Fusions also include larger polypeptides, or even entire proteins, such as the green fluorescent protein (GFP) chromophore-containing proteins having particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

The term “functional nucleic acid molecule” refers to a nucleic acid molecule that, upon introduction into a host cell or expression in a host cell, specifically interferes with expression of a protein. In general, functional nucleic acid molecules have the capacity to reduce expression of a protein by directly interacting with a transcript that encodes the protein. Ribozymes, antisense nucleic acid molecules, and siRNA molecules, including shRNA molecules, short RNAs (typically less than 400 bases in length), and micro-RNAs (miRNAs) constitute exemplary functional nucleic acid molecules.

The function of a gene encoding a protein is said to be ‘reduced’ when that gene has been modified, for example, by deletion, insertion, mutation or substitution of one or more nucleotides, such that the modified gene encodes a protein which has at least 20% to 50% lower activity, in particular aspects, at least 40% lower activity or at least 50% lower activity, when measured in a standard assay, as compared to the protein encoded by the corresponding gene without such modification. The function of a gene encoding a protein is said to be ‘eliminated’ when the gene has been modified, for example, by deletion, insertion, mutation or substitution of one or more nucleotides, such that the modified gene encodes a protein which has at least 90% to 99% lower activity, in particular aspects, at least 95% lower activity or at least 99% lower activity, when measured in a standard assay, as compared to the protein encoded by the corresponding gene without such modification.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs cotranslationally in the lumen of the ER and continues in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.” Abbreviations used herein are of common usage in the art, see, e.g., abbreviations of sugars, above. Other common abbreviations include “PNGase”, or “glycanase” or “glucosidase” which all refer to peptide N-glycosidase F (EC 3.2.2.18).

Unless otherwise indicated, a “nucleic acid molecule comprising SEQ ID NO:X” refers to a nucleic acid molecule, at least a portion of which has either (i) the sequence of SEQ ID NO:X, or (ii) a sequence complementary to SEQ ID NO:X. The choice between the two is dictated by the context. For instance, if the nucleic acid molecule is used as a probe, the choice between the two is dictated by the requirement that the probe be complementary to the desired target.

An “isolated” or “substantially pure” nucleic acid molecule or polynucleotide (e.g., an RNA, DNA or a mixed polymer) comprising the MET1, MET3, MET4, MET6, MET7, MET8, MET10, MET14, MET16, MET17, MET19, MET22, MET27, or MET28 gene or fragment thereof is one which is substantially separated from other cellular components that naturally accompany the native polynucleotide in its natural host cell, e.g., ribosomes, polymerases, and genomic sequences with which it is naturally associated. The term embraces a nucleic acid molecule or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid molecule or polynucleotide so described has itself been physically removed from its native environment. For instance, an endogenous nucleic acid sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g., by homologous recombination) for the native promoter of a gene in the genome of a human cell, such that this gene has an altered expression pattern. This gene would now become “isolated” because it is separated from at least some of the sequences that naturally flank it.

A nucleic acid molecule is also considered “isolated” if it contains any modifications that do not naturally occur to the corresponding nucleic acid molecule in a genome. For instance, an endogenous coding sequence is considered “isolated” if it contains an insertion, deletion or a point mutation introduced artificially, e.g., by human intervention. An “isolated nucleic acid molecule” also includes a nucleic acid molecule integrated into a host cell chromosome at a heterologous site, a nucleic acid molecule construct present as an episome. Moreover, an “isolated nucleic acid molecule” can be substantially free of other cellular material, or substantially free of culture medium when produced by recombinant techniques, or substantially free of chemical precursors or other chemicals when chemically synthesized.

As used herein, the phrase “degenerate variant” of nucleic acid sequence comprising the MET1, MET3, MET4, MET6, MET7, MET8, MET10, MET14, MET16, MET17, MET19, MET22, MET27, or MET28 gene or fragment thereof encompasses nucleic acid sequences that can be translated, according to the standard genetic code, to provide an amino acid sequence identical to that translated from the reference nucleic acid sequence.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art that can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences (Pearson, 1990, herein incorporated by reference). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

The term “substantial homology” or “substantial similarity,” when referring to a nucleic acid molecule or fragment thereof, indicates that, when optimally aligned with appropriate nucleotide insertions or deletions with another nucleic acid molecule (or its complementary strand), there is nucleotide sequence identity in at least about 50%, more preferably 60% of the nucleotide bases, usually at least about 70%, more usually at least about 80%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid molecule or fragment thereof hybridizes to another nucleic acid molecule, to a strand of another nucleic acid molecule, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acid molecules, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., supra, page 9.51, hereby incorporated by reference. For purposes herein, “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled artisan that hybridization at 65° C. will occur at different rates depending on a number of factors including the length and percent identity of the sequences which are hybridizing.

The term “mutated” when applied to nucleic acid sequences comprising the MET1, MET3, MET4, MET6, MET7, MET8, MET10, MET14, MET16, MET17, MET19, MET22, MET27, or MET28 gene or fragment thereof means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product. See, e.g., Leung, D. W., et al., Technique, 1, pp. 11-15 (1989) and Caldwell, R. C. & Joyce G. F., PCR Methods Applic., 2, pp. 28-33 (1992)); and “oligonucleotide-directed mutagenesis” (a process which enables the generation of site-specific mutations in any cloned DNA segment of interest. See, e.g., Reidhaar-Olson, J. F. & Sauer, R. T., et al., Science, 241, pp. 53-57 (1988)).

The term “isolated protein” or “isolated polypeptide” is a protein or polypeptide such as Met1p, Met3p, Met4p, Met6p, Met7p, Met8p, Met10p, Met14p, Met16p, Met17p, Met19p, Met22p, Met27p, or Met28p that by virtue of its origin or source of derivation (1) is not associated with naturally associated components that accompany it in its native state, (2) when it exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well-known in the art. As thus defined, “isolated” does not necessarily require that the protein, polypeptide, peptide or oligopeptide so described has been physically removed from its native environment.

The term “polypeptide fragment” as used herein refers to a polypeptide derived from Met1p, Met3p, Met4p, Met6p, Met7p, Met8p, Met10p, Met14p, Met16p, Met17p, Met19p, Met22p, Met27p, or Met28p that has an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to Met1p, Met3p, Met4p, Met6p, Met7p, Met8p, Met10p, Met14p, Met16p, Met17p, Met19p, Met22p, Met27p, or Met28p polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well-known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well-known in the art. See Ausubel et al., Current Potocols in Molecular Biology, Greene Publishing Associates (1992, and supplement sto 2002) hereby incorporated by reference.

A “polypeptide mutant” or “mutein” refers to a Met1p, Met3p, Met4p, Met6p, Met7p, Met8p, Met10p, Met14p, Met16p, Met17p, Met19p, Met22p, Met27p, or Met28p polypeptide whose sequence contains an insertion, duplication, deletion, rearrangement or substitution of one or more amino acids compared to the amino acid sequence of a native or wild type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A Met1p, Met3p, Met4p, Met6p, Met7p, Met8p, Met10p, Met14p, Met16p, Met17p, Met19p, Met22p, Met27p, or Met28p mutein has at least 70% overall sequence homology to its wild-type counterpart. Even more preferred are muteins having 80%, 85% or 90% overall sequence homology to the wild-type protein. In an even more preferred embodiment, a mutein exhibits 95% sequence identity, even more preferably 97%, even more preferably 98% and even more preferably 99% overall sequence identity. Sequence homology may be measured by any common sequence analysis algorithm, such as Gap or Bestfit.

Preferred amino acid substitutions are those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethylmethionine, β-N-acetylmethionine, O-phosphoserine, N-acetylserine, N-formylmethionine, 3-methylhistidine, 5-hydroxymethionine, s-N-methylmethionine, and other similar amino acids and imino acids (e.g., 4-hydroxyproline). In the polypeptide notation used herein, the left-hand direction is the amino terminal direction and the right hand direction is the carboxy-terminal direction, in accordance with standard usage and convention.

A Met1p, Met3p, Met4p, Met6p, Met7p, Met8p, Met10p, Met14p, Met16p, Met17p, Met19p, Met22p, Met27p, or Met28p protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences). In a preferred embodiment, a homologous protein is one that exhibits 60% sequence homology to the wild type protein, more preferred is 70% sequence homology. Even more preferred are homologous proteins that exhibit 80%, 85% or 90% sequence homology to the wild type protein. In a yet more preferred embodiment, a homologous protein exhibits 95%, 97%, 98% or 99% sequence identity. As used herein, homology between two regions of amino acid sequence (especially with respect to predicted structural similarities) is interpreted as implying similarity in function.

When “homologous” is used in reference to Met1p, Met3p, Met4p, Met6p, Met7p, Met8p, Met10p, Met14p, Met16p, Met17p, Met19p, Met22p, Met27p, or Met28p proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for Met1p, Met3p, Met4p, Met6p, Met7p, Met8p, Met10p, Met14p, Met16p, Met17p, Met19p, Met22p, Met27p, or Met28p polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A preferred algorithm when comparing a inhibitory molecule sequence to a database containing a large number of sequences from different organisms is the computer program BLAST (Altschul, S. F. et al. (1990) J. Mol. Biol. 215:403-410; Gish and States (1993) Nature Genet. 3:266-272; Madden, T. L. et al. (1996) Meth. Enzymol. 266:131-141; Altschul, S. F. et al. (1997) Nucleic Acids Res. 25:3389-3402; Zhang, J. and Madden, T. L. (1997) Genome Res. 7:649-656), especially blastp or tblastn (Altschul et al., 1997). Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, herein incorporated by reference). For example, percent sequence identity between amino acid sequences can be determined using FASTA with its default parameters (a word size of 2 and the PAM250 scoring matrix), as provided in GCG Version 6.1, herein incorporated by reference.

As used herein, the terms “antibody,” “immunoglobulin,” “immunoglobulins”, “IgG1”, “antibodies”, and “immunoglobulin molecule” are used interchangeably. Each immunoglobulin molecule has a unique structure that allows it to bind its specific antigen, but all immunoglobulins have the same overall structure as described herein. The basic immunoglobulin structural unit is known to comprise a tetramer of subunits. Each tetramer has two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Light chains are classified as either kappa or lambda. Heavy chains are classified as gamma, mu, alpha, delta, or epsilon, and define the antibody's isotype as IgG, IgM, IgA, IgD, and IgE, respectively.

The light and heavy chains are subdivided into variable regions and constant regions (See generally, Fundamental Immunology (Paul, W., ed., 2nd ed. Raven Press, N.Y., 1989), Ch. 7. The variable regions of each light/heavy chain pair form the antibody binding site. Thus, an intact antibody has two binding sites. Except in bifunctional or bispecific immunoglobulins, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. The terms include naturally occurring forms, as well as fragments and derivatives. Included within the scope of the term are classes of immunoglobulins (Igs), namely, IgG, IgA, IgE, IgM, and IgD. Also included within the scope of the terms are the subtypes of IgGs, namely, IgG1, IgG2, IgG3, and IgG4. The term is used in the broadest sense and includes single monoclonal immunoglobulins (including agonist and antagonist immunoglobulins) as well as antibody compositions which will bind to multiple epitopes or antigens. The terms specifically cover monoclonal immunoglobulins (including full length monoclonal immunoglobulins), polyclonal immunoglobulins, multispecific immunoglobulins (for example, bispecific immunoglobulins), and antibody fragments so long as they contain or are modified to contain at least the portion of the CH₂ domain of the heavy chain immunoglobulin constant region which comprises an N-linked glycosylation site of the CH₂ domain, or a variant thereof. The C_{H2} domain of each heavy chain of an antibody contains a single site for N-linked glycosylation: this is usually at the asparagine residue 297 (Asn-297) (Kabat et al., Sequences of proteins of immunological interest, Fifth Ed., U.S. Department of Health and Human Services, NIH Publication No. 91-3242). Included within the terms are molecules comprising only the Fc region, such as immunoadhesins (U.S. Published Patent Application No. 20040136986), Fc fusions, and antibody-like molecules.

The term “monoclonal antibody” (mAb) as used herein refers to an antibody obtained from a population of substantially homogeneous immunoglobulins, i.e., the individual immunoglobulins comprising the population are identical except for possible naturally occurring mutations that may be present in minor amounts. Monoclonal immunoglobulins are highly specific, being directed against a single antigenic site. Furthermore, in contrast to conventional (polyclonal) antibody preparations which typically include different immunoglobulins directed against different determinants (epitopes), each mAb is directed against a single determinant on the antigen. In addition to their specificity, monoclonal immunoglobulins are advantageous in that they can be synthesized by hybridoma culture, uncontaminated by other immunoglobulins. The term “monoclonal” indicates the character of the antibody as being obtained from a substantially homogeneous population of immunoglobulins, and is not to be construed as requiring production of the antibody by any particular method. For example, the monoclonal immunoglobulins to be used in accordance with the present invention may be made by the hybridoma method first described by Kohler et al., Nature, 256:495 (1975), or may be made by recombinant DNA methods (See, for example, U.S. Pat. No. 4,816,567 to Cabilly et al.).

The term “fragments” within the scope of the terms “antibody” or “immunoglobulin” include those produced by digestion with various proteases, those produced by chemical cleavage and/or chemical dissociation and those produced recombinantly, so long as the fragment remains capable of specific binding to a target molecule. Among such fragments are Fc, Fab, Fab′, Fv, F(ab′)₂, and single chain Fv (scFv) fragments. Hereinafter, the term “immunoglobulin” also includes the term “fragments” as well.

The term “Fc” fragment refers to the ‘fragment crystallized’ C-terminal region of the antibody containing the CH₂ and CH₃ domains (FIG. 1). The term “Fab” fragment refers to the ‘fragment antigen binding’ region of the antibody containing the V_{H}, C_{H}1, V_{L} and C_{L} domains.

Immunoglobulins further include immunoglobulins or fragments that have been modified in sequence but remain capable of specific binding to a target molecule, including: interspecies chimeric and humanized immunoglobulins; antibody fusions; heteromeric antibody complexes and antibody fusions, such as diabodies (bispecific immunoglobulins), single-chain diabodies, and intrabodies (See, for example, Intracellular Immunoglobulins: Research and Disease Applications, (Marasco, ed., Springer-Verlag New York, Inc., 1998).

The term “catalytic antibody” refers to immunoglobulin molecules that are capable of catalyzing a biochemical reaction. Catalytic immunoglobulins are well known in the art and have been described in U.S. Pat. Nos. 7,205,136; 4,888,281; 5,037,750 to Schochetman et al., U.S. Pat. Nos. 5,733,757; 5,985,626; and 6,368,839 to Barbas, III et al.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting in any manner.

### DETAILED DESCRIPTION OF THE INVENTION

The present invention provides methods and vectors for integrating heterologous DNA into the MET1, MET3, MET4, MET6, MET7, MET8, MET10, MET14, MET16, MET17, MET19, MET22, MET27, or MET28 locus. The present invention further provides the use of a nucleic acid sequence encoding the enzyme encoded by any one of the loci for use as a selectable marker in methods in which a plasmid vector containing the nucleic acid sequence is transformed into the host cell that is auxotrophic for methionine because the gene in the genome encoding the enzyme has been deleted or disrupted. Table 1 provides a description of several of the enzymes in the methionine biosynthetic pathway.

TABLE 1
Auxotrophic Markers

| Locus | Description |
|---|---|
| MET1 | S-adenosyl-L-methionine uroporphyrinogen III transmethylase, involved |
| | in sulfate assimilation, methionine metabolism, and siroheme biosynthesis. |
| | Null mutant is viable, and is a methionine auxotroph |
| MET2 | L-homoserine-O-acetyltransferase, catalyzes the conversion of homoserine |
| | to O-acetyl homoserine which is the first step of the methionine |
| | biosynthetic pathway. Null mutant is viable, and is a methionine |
| | auxotroph. |
| MET3 | ATP sulfurylase, catalyzes the primary step of intracellular sulfate |
| | activation, essential for assimilatory reduction of sulfate to sulfide, |
| | involved in methionine metabolism. Null mutant is viable, and is a |
| | methionine auxotroph. |
| MET4 | Leucine-zipper transcriptional activator, responsible for the regulation of |
| | the sulfur amino acid pathway, requires different combinations of the |
| | auxiliary factors Cbf1p, Met28p, Met31p and Met32p. Null mutant is |
| | viable, is methionine auxotroph, and shows increased acetaldehyde |
| | sensitivity. |
| MET5 | Sulfite reductase beta subunit, involved in amino acid biosynthesis, |
| | transcription repressed by methionine. Loss of function mutants are |
| | methionine requiring and sensitive to the cell wall perturbing agent |
| | calcoflour white. |
| MET6 | Cobalamin-independent methionine synthase, involved in amino acid |
| | biosynthesis; requires a minimum of two glutamates on the |
| | methyltetrahydrofolate substrate, similar to bacterial metE homologs. Null |
| | mutant is viable, and is a methionine auxotroph. |
| MET7 | Folylpolyglutamate synthetase, catalyzes extension of the glutamate chains |
| | of the folate coenzymes, required for methionine synthesis and for |
| | maintenance of mitochondrial DNA, present in both the cytoplasm and |
| | mitochondria. Null mutant is viable, requires methionine for growth, and is |
| | respiration-deficient. |
| MET8 | Bifunctional dehydrogenase and ferrochelatase, involved in the |
| | biosynthesis of siroheme; also involved in the expression of PAPS |
| | reductase and sulfite reductase. Null mutant is viable, and is a methionine |
| | auxotroph. |
| MET10 | Subunit alpha of assimilatory sulfite reductase, which is responsible for the |
| | conversion of sulfite into sulfide. Null mutant is a methionine auxotroph. |
| MET14 | Adenylylsulfate kinase, required for sulfate assimilation and involved in |
| | methionine metabolism. Null mutant is viable, and is a methionine |
| | auxotroph. |
| MET16 | 3′-phosphoadenylsulfate reductase, reduces 3′-phosphoadenylyl sulfate to |
| | adenosine-3′,5′-bisphosphate and free sulfite using reduced thioredoxin as |
| | cosubstrate, involved in sulfate assimilation and methionine metabolism. |
| | Null mutant is viable, and is a methionine auxotroph. |
| MET17 | O-acetyl homoserine-O-acetyl serine sulfhydrylase, required for sulfur |
| | amino acid synthesis. Null mutant is viable, methionine auxotroph, |
| | becomes darkly pigmented in the presence of Pb2+ ions, resistant to |
| | methylmercury, and exhibits increased levels of H2S |
| MET18 | DNA repair and TFIIH regulator, required for both nucleotide excision |
| | repair (NER) and RNA polymerase II (RNAP II) transcription; possible |
| | role in assembly of a multiprotein complex(es) required for NER and |
| | RNAP II transcription. Null mutant is viable but is temperature-sensitive, |
| | defective in ability to remove UV_induced dimers from nuclear DNA, and |
| | shows enhanced UV-induced mutations; extracts from mutant exhibit |
| | thermolabile defect in RNA Pol II transcription; methionine auxotroph. |
| MET19 | Glucose-6-phosphate dehydrogenase (G6PD), catalyzes the first step of the |
| | pentose phosphate pathway; involved in adapting to oxidatve stress; |
| | homolog of the human G6PD which is deficient in patients with hemolytic |
| | anemia. Null mutant is viable, sensitive to oxidizing agents; methionine |
| | requiring |
| MET22 | Bisphosphate-3′-nucleotidase, involved in salt tolerance and methionine |
| | biogenesis; dephosphorylates 3′-phosphoadenosine-5′-phosphate and 3′- |
| | phosphoadenosine-5′-phosphosulfate, intermediates of the sulfate |
| | assimilation pathway. Methionine requiring; lacks 3′- |
| | phosphoadenylylsulfate (PAPS) reductase activity; unable to grow on |
| | sulfate as sole sulfur source; overexpression confers lithium resistance; |
| | pAp accumulation in met22 mutants (or under MET22 inhibition) inhibits |
| | the 5′−>3′ exoribonucleases Xrn1p and Rat1p. |
| MET27 | ATP-binding protein that is a subunit of the homotypic vacuole fusion and |
| | vacuole protein sorting (HOPS) complex; essential for membrane docking |
| | and fusion at both the Golgi-to-endosome and endosome-to-vacuole stages |
| | of protein transport. Null mutant is temperature sensitive, has defective |
| | vacuolar morphology and protein localization, and is methionine auxotroph |
| | Is also called VPS33. |
| MET28 | Transcriptional activator in the Cbf1p-Met4p-Met28p complex, |
| | participates in the regulation of sulfur metabolism. Null mutant is viable |
| | but is a methionine-auxotroph and resistant to toxic analogs of sulfate. |

The genome of Pichia pastoris was sequenced and annotated by Schutter et al. (Nature Biotechnol. 27: 561-569 (2009)) and Mattanovitch et al., (Microbial Cell Factories 8: 53-56 (2009)). The nucleic acid sequences for the MET1, MET3, MET4, MET6, MET7, MET8, MET10, MET14, MET16, MET17, MET19, MET22, MET27, and MET28 loci are provided in SEQ ID NO:1, 3, 5, 7, 9, 11, 13, 15, 17, 19, 21, 23, 25, and 27, respectively.

Provided herein is an isolated nucleic acid molecule having a nucleic acid sequence comprising or consisting of a wild-type P. pastoris MET1 gene sequence (SEQ ID NO:1), and homologs, variants and derivatives thereof. Further provided is a nucleic acid molecule comprising or consisting of a sequence which is a degenerate variant of the wild-type P. pastoris MET1 gene. In particular aspects, the nucleic acid molecule comprises or consists of a sequence which is a variant of the P. pastoris MET1 gene (SEQ ID NO: 1) having at least 65% identity to the wild-type gene or to a nucleotide sequence comprising at least 25, 50, 75, 100, 125, 150, 175, or 200 contiguous nucleotides of SEQ ID NO:1. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:1. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:1. The nucleic acid molecule encodes a polypeptide having the amino acid sequence of SEQ ID NO:2. Also provided is a nucleic acid molecule encoding a polypeptide sequence that is at least 65% identical to an amino acid sequence comprising the amino acid sequence of SEQ ID NO:2 or an amino acid sequence comprising at least 25, 50, 75, 100, 125, 150, 175, or 200 contiguous amino acids of SEQ ID NO:2. Typically the nucleic acid molecule encodes a polypeptide sequence of at least 70%, 75% or 80% identity to an amino acid sequence comprising the amino acid sequence of SEQ ID NO:2 or an amino acid sequence comprising at least 25, 50, 75, 100, 125, 150, 175, or 200 contiguous amino acids of SEQ ID NO:2. In further aspects, the encoded polypeptide is 85%, 90% or 95% identical to an amino acid sequence comprising the amino acid sequence of SEQ ID NO:2 or an amino acid sequence comprising at least 25, 50, 75, 100, 125, 150, 175, or 200 contiguous amino acids of SEQ ID NO:2 or 98%, 99%, 99.9% identical to an amino acid sequence comprising the amino acid sequence of SEQ ID NO:2 or an amino acid sequence comprising at least 25, 50, 75, 100, 125, 150, 175, or 200 contiguous amino acids of SEQ ID NO:2.

Provided herein is an isolated nucleic acid molecule having a nucleic acid sequence comprising or consisting of a wild-type P. pastoris MET3 gene sequence (SEQ ID NO:3), and homologs, variants and derivatives thereof. Further provided is a nucleic acid molecule comprising or consisting of a sequence which is a degenerate variant of the wild-type P. pastoris MET3 gene. In particular aspects, the nucleic acid molecule comprises or consists of a sequence which is a variant of the P. pastoris MET3 gene (SEQ ID NO: 3) having at least 65% identity to the wild-type gene or to a nucleotide sequence comprising at least 25, 50, 75, 100, 125, 150, 175, or 200 contiguous nucleotides of SEQ ID NO:3. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:3. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:3. The nucleic acid molecule encodes a polypeptide having the amino acid sequence of SEQ ID NO:4. Also provided is a nucleic acid molecule encoding a polypeptide sequence that is at least 65% identical to an amino acid sequence comprising the amino acid sequence of SEQ ID NO:4 or an amino acid sequence comprising at least 25, 50, 75, 100, 125, 150, 175, or 200 contiguous amino acids of SEQ ID NO:4. Typically the nucleic acid molecule encodes a polypeptide sequence of at least 70%, 75% or 80% identity to an amino acid sequence comprising the amino acid sequence of SEQ ID NO:4 or an amino acid sequence comprising at least 25, 50, 75, 100, 125, 150, 175, or 200 contiguous amino acids of SEQ ID NO:4. In further aspects, the encoded polypeptide is 85%, 90% or 95% identical to an amino acid sequence comprising the amino acid sequence of SEQ ID NO:4 or an amino acid sequence comprising at least 25, 50, 75, 100, 125, 150, 175, or 200 contiguous amino acids of SEQ ID NO:4 or 98%, 99%, 99.9% identical to an amino acid sequence comprising the amino acid sequence of SEQ ID NO:4 or an amino acid sequence comprising at least 25, 50, 75, 100, 125, 150, 175, or 200 contiguous amino acids of SEQ ID NO:4.

Provided herein is an isolated nucleic acid molecule having a nucleic acid sequence comprising or consisting of a wild-type P. pastoris MET4 gene sequence (SEQ ID NO:5), and homologs, variants and derivatives thereof. Further provided is a nucleic acid molecule comprising or consisting of a sequence which is a degenerate variant of the wild-type P. pastoris MET4 gene. In particular aspects, the nucleic acid molecule comprises or consists of a sequence which is a variant of the P. pastoris MET4 gene (SEQ ID NO: 5) having at least 65% identity to the wild-type gene or to a nucleotide sequence comprising at least 25, 50, 75, 100, 125, 150, 175, or 200 contiguous nucleotides of SEQ ID NO:5. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:5. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:5. The nucleic acid molecule encodes a polypeptide having the amino acid sequence of SEQ ID NO:6. Also provided is a nucleic acid molecule encoding a polypeptide sequence that is at least 65% identical to an amino acid sequence comprising the amino acid sequence of SEQ ID NO:6 or an amino acid sequence comprising at least 25, 50, 75, 100, 125, 150, 175, or 200 contiguous amino acids of SEQ ID NO:6. Typically the nucleic acid molecule encodes a polypeptide sequence of at least 70%, 75% or 80% identity to an amino acid sequence comprising the amino acid sequence of SEQ ID NO:6 or an amino acid sequence comprising at least 25, 50, 75, 100, 125, 150, 175, or 200 contiguous amino acids of SEQ ID NO:6. In further aspects, the encoded polypeptide is 85%, 90% or 95% identical to an amino acid sequence comprising the amino acid sequence of SEQ ID NO:6 or an amino acid sequence comprising at least 25, 50, 75, 100, 125, 150, 175, or 200 contiguous amino acids of SEQ ID NO:6 or 98%, 99%, 99.9% identical to an amino acid sequence comprising the amino acid sequence of SEQ ID NO:6 or an amino acid sequence comprising at least 25, 50, 75, 100, 125, 150, 175, or 200 contiguous amino acids of SEQ ID NO:6.

Provided herein is an isolated nucleic acid molecule having a nucleic acid sequence comprising or consisting of a wild-type P. pastoris MET6 gene sequence (SEQ ID NO:7), and homologs, variants and derivatives thereof. Further provided is a nucleic acid molecule comprising or consisting of a sequence which is a degenerate variant of the wild-type P. pastoris MET6 gene. In particular aspects, the nucleic acid molecule comprises or consists of a sequence which is a variant of the P. pastoris MET6 gene (SEQ ID NO: 7) having at least 65% identity to the wild-type gene or to a nucleotide sequence comprising at least 25, 50, 75, 100, 125, 150, 175, or 200 contiguous nucleotides of SEQ ID NO:7. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:7. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:7. The nucleic acid molecule encodes a polypeptide having the amino acid sequence of SEQ ID NO:8. Also provided is a nucleic acid molecule encoding a polypeptide sequence that is at least 65% identical to an amino acid sequence comprising the amino acid sequence of SEQ ID NO:8 or an amino acid sequence comprising at least 25, 50, 75, 100, 125, 150, 175, or 200 contiguous amino acids of SEQ ID NO:8. Typically the nucleic acid molecule encodes a polypeptide sequence of at least 70%, 75% or 80% identity to an amino acid sequence comprising the amino acid sequence of SEQ ID NO:8 or an amino acid sequence comprising at least 25, 50, 75, 100, 125, 150, 175, or 200 contiguous amino acids of SEQ ID NO:8. In further aspects, the encoded polypeptide is 85%, 90% or 95% identical to an amino acid sequence comprising the amino acid sequence of SEQ ID NO:8 or an amino acid sequence comprising at least 25, 50, 75, 100, 125, 150, 175, or 200 contiguous amino acids of SEQ ID NO:8 or 98%, 99%, 99.9% identical to an amino acid sequence comprising the amino acid sequence of SEQ ID NO:8 or an amino acid sequence comprising at least 25, 50, 75, 100, 125, 150, 175, or 200 contiguous amino acids of SEQ ID NO:8.

Provided herein is an isolated nucleic acid molecule having a nucleic acid sequence comprising or consisting of a wild-type P. pastoris MET7 gene sequence (SEQ ID NO:9), and homologs, variants and derivatives thereof. Further provided is a nucleic acid molecule comprising or consisting of a sequence which is a degenerate variant of the wild-type P. pastoris MET7 gene. In particular aspects, the nucleic acid molecule comprises or consists of a sequence which is a variant of the P. pastoris MET7 gene (SEQ ID NO: 9) having at least 65% identity to the wild-type gene or to a nucleotide sequence comprising at least 25, 50, 75, 100, 125, 150, 175, or 200 contiguous nucleotides of SEQ ID NO:9. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:9. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:9. The nucleic acid molecule encodes a polypeptide having the amino acid sequence of SEQ ID NO:10. Also provided is a nucleic acid molecule encoding a polypeptide sequence that is at least 65% identical to an amino acid sequence comprising the amino acid sequence of SEQ ID NO:10 or an amino acid sequence comprising at least 25, 50, 75, 100, 125, 150, 175, or 200 contiguous amino acids of SEQ ID NO:10. Typically the nucleic acid molecule encodes a polypeptide sequence of at least 70%, 75% or 80% identity to an amino acid sequence comprising the amino acid sequence of SEQ ID NO:10 or an amino acid sequence comprising at least 25, 50, 75, 100, 125, 150, 175, or 200 contiguous amino acids of SEQ ID NO:10. In further aspects, the encoded polypeptide is 85%, 90% or 95% identical to an amino acid sequence comprising the amino acid sequence of SEQ ID NO:10 or an amino acid sequence comprising at least 25, 50, 75, 100, 125, 150, 175, or 200 contiguous amino acids of SEQ ID NO:10 or 98%, 99%, 99.9% identical to an amino acid sequence comprising the amino acid sequence of SEQ ID NO:10 or an amino acid sequence comprising at least 25, 50, 75, 100, 125, 150, 175, or 200 contiguous amino acids of SEQ ID NO:10.

Provided herein is an isolated nucleic acid molecule having a nucleic acid sequence comprising or consisting of a wild-type P. pastoris MET8 gene sequence (SEQ ID NO:11), and homologs, variants and derivatives thereof. Further provided is a nucleic acid molecule comprising or consisting of a sequence which is a degenerate variant of the wild-type P. pastoris MET8 gene. In particular aspects, the nucleic acid molecule comprises or consists of a sequence which is a variant of the P. pastoris MET8 gene (SEQ ID NO: 11) having at least 65% identity to the wild-type gene or to a nucleotide sequence comprising at least 25, 50, 75, 100, 125, 150, 175, or 200 contiguous nucleotides of SEQ ID NO:11. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:11. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:11. The nucleic acid molecule encodes a polypeptide having the amino acid sequence of SEQ ID NO:12. Also provided is a nucleic acid molecule encoding a polypeptide sequence that is at least 65% identical to an amino acid sequence comprising the amino acid sequence of SEQ ID NO:12 or an amino acid sequence comprising at least 25, 50, 75, 100, 125, 150, 175, or 200 contiguous amino acids of SEQ ID NO:12. Typically the nucleic acid molecule encodes a polypeptide sequence of at least 70%, 75% or 80% identity to an amino acid sequence comprising the amino acid sequence of SEQ ID NO:12 or an amino acid sequence comprising at least 25, 50, 75, 100, 125, 150, 175, or 200 contiguous amino acids of SEQ ID NO:12. In further aspects, the encoded polypeptide is 85%, 90% or 95% identical to an amino acid sequence comprising the amino acid sequence of SEQ ID NO:12 or an amino acid sequence comprising at least 25, 50, 75, 100, 125, 150, 175, or 200 contiguous amino acids of SEQ ID NO:12 or 98%, 99%, 99.9% identical to an amino acid sequence comprising the amino acid sequence of SEQ ID NO:12 or an amino acid sequence comprising at least 25, 50, 75, 100, 125, 150, 175, or 200 contiguous amino acids of SEQ ID NO:12.

Provided herein is an isolated nucleic acid molecule having a nucleic acid sequence comprising or consisting of a wild-type P. pastoris MET10 gene sequence (SEQ ID NO:13), and homologs, variants and derivatives thereof. Further provided is a nucleic acid molecule comprising or consisting of a sequence which is a degenerate variant of the wild-type P. pastoris MET10 gene. In particular aspects, the nucleic acid molecule comprises or consists of a sequence which is a variant of the P. pastoris MET10 gene (SEQ ID NO: 13) having at least 65% identity to the wild-type gene or to a nucleotide sequence comprising at least 25, 50, 75, 100, 125, 150, 175, or 200 contiguous nucleotides of SEQ ID NO:13. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:13. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:13. The nucleic acid molecule encodes a polypeptide having the amino acid sequence of SEQ ID NO:14. Also provided is a nucleic acid molecule encoding a polypeptide sequence that is at least 65% identical to an amino acid sequence comprising the amino acid sequence of SEQ ID NO:14 or an amino acid sequence comprising at least 25, 50, 75, 100, 125, 150, 175, or 200 contiguous amino acids of SEQ ID NO:14. Typically the nucleic acid molecule encodes a polypeptide sequence of at least 70%, 75% or 80% identity to an amino acid sequence comprising the amino acid sequence of SEQ ID NO:14 or an amino acid sequence comprising at least 25, 50, 75, 100, 125, 150, 175, or 200 contiguous amino acids of SEQ ID NO:14. In further aspects, the encoded polypeptide is 85%, 90% or 95% identical to an amino acid sequence comprising the amino acid sequence of SEQ ID NO:14 or an amino acid sequence comprising at least 25, 50, 75, 100, 125, 150, 175, or 200 contiguous amino acids of SEQ ID NO:14 or 98%, 99%, 99.9% identical to an amino acid sequence comprising the amino acid sequence of SEQ ID NO:14 or an amino acid sequence comprising at least 25, 50, 75, 100, 125, 150, 175, or 200 contiguous amino acids of SEQ ID NO:14.

Provided herein is an isolated nucleic acid molecule having a nucleic acid sequence comprising or consisting of a wild-type P. pastoris MET14 gene sequence (SEQ ID NO:15), and homologs, variants and derivatives thereof. Further provided is a nucleic acid molecule comprising or consisting of a sequence which is a degenerate variant of the wild-type P. pastoris MET14 gene. In particular aspects, the nucleic acid molecule comprises or consists of a sequence which is a variant of the P. pastoris MET14 gene (SEQ ID NO: 15) having at least 65% identity to the wild-type gene or to a nucleotide sequence comprising at least 25, 50, 75, 100, 125, 150, 175, or 200 contiguous nucleotides of SEQ ID NO:15. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:15. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:15. The nucleic acid molecule encodes a polypeptide having the amino acid sequence of SEQ ID NO:16. Also provided is a nucleic acid molecule encoding a polypeptide sequence that is at least 65% identical to an amino acid sequence comprising the amino acid sequence of SEQ ID NO:16 or an amino acid sequence comprising at least 25, 50, 75, 100, 125, 150, 175, or 200 contiguous amino acids of SEQ ID NO:16. Typically the nucleic acid molecule encodes a polypeptide sequence of at least 70%, 75% or 80% identity to an amino acid sequence comprising the amino acid sequence of SEQ ID NO:16 or an amino acid sequence comprising at least 25, 50, 75, 100, 125, 150, 175, or 200 contiguous amino acids of SEQ ID NO:16. In further aspects, the encoded polypeptide is 85%, 90% or 95% identical to an amino acid sequence comprising the amino acid sequence of SEQ ID NO:16 or an amino acid sequence comprising at least 25, 50, 75, 100, 125, 150, 175, or 200 contiguous amino acids of SEQ ID NO:16 or 98%, 99%, 99.9% identical to an amino acid sequence comprising the amino acid sequence of SEQ ID NO:16 or an amino acid sequence comprising at least 25, 50, 75, 100, 125, 150, 175, or 200 contiguous amino acids of SEQ ID NO:16.

Provided herein is an isolated nucleic acid molecule having a nucleic acid sequence comprising or consisting of a wild-type P. pastoris MET16 gene sequence (SEQ ID NO:17), and homologs, variants and derivatives thereof. Further provided is a nucleic acid molecule comprising or consisting of a sequence which is a degenerate variant of the wild-type P. pastoris MET16 gene. In particular aspects, the nucleic acid molecule comprises or consists of a sequence which is a variant of the P. pastoris MET16 gene (SEQ ID NO: 17) having at least 65% identity to the wild-type gene or to a nucleotide sequence comprising at least 25, 50, 75, 100, 125, 150, 175, or 200 contiguous nucleotides of SEQ ID NO:17. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:17. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:17. The nucleic acid molecule encodes a polypeptide having the amino acid sequence of SEQ ID NO:18. Also provided is a nucleic acid molecule encoding a polypeptide sequence that is at least 65% identical to an amino acid sequence comprising the amino acid sequence of SEQ ID NO:18 or an amino acid sequence comprising at least 25, 50, 75, 100, 125, 150, 175, or 200 contiguous amino acids of SEQ ID NO:18. Typically the nucleic acid molecule encodes a polypeptide sequence of at least 70%, 75% or 80% identity to an amino acid sequence comprising the amino acid sequence of SEQ ID NO:18 or an amino acid sequence comprising at least 25, 50, 75, 100, 125, 150, 175, or 200 contiguous amino acids of SEQ ID NO:18. In further aspects, the encoded polypeptide is 85%, 90% or 95% identical to an amino acid sequence comprising the amino acid sequence of SEQ ID NO:18 or an amino acid sequence comprising at least 25, 50, 75, 100, 125, 150, 175, or 200 contiguous amino acids of SEQ ID NO:18 or 98%, 99%, 99.9% identical to an amino acid sequence comprising the amino acid sequence of SEQ ID NO:18 or an amino acid sequence comprising at least 25, 50, 75, 100, 125, 150, 175, or 200 contiguous amino acids of SEQ ID NO:18.

Provided herein is an isolated nucleic acid molecule having a nucleic acid sequence comprising or consisting of a wild-type P. pastoris MET17 gene sequence (SEQ ID NO:19), and homologs, variants and derivatives thereof. Further provided is a nucleic acid molecule comprising or consisting of a sequence which is a degenerate variant of the wild-type P. pastoris MET17 gene. In particular aspects, the nucleic acid molecule comprises or consists of a sequence which is a variant of the P. pastoris MET17 gene (SEQ ID NO: 19) having at least 65% identity to the wild-type gene or to a nucleotide sequence comprising at least 25, 50, 75, 100, 125, 150, 175, or 200 contiguous nucleotides of SEQ ID NO:19. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:19. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:19. The nucleic acid molecule encodes a polypeptide having the amino acid sequence of SEQ ID NO:20. Also provided is a nucleic acid molecule encoding a polypeptide sequence that is at least 65% identical to an amino acid sequence comprising the amino acid sequence of SEQ ID NO:20 or an amino acid sequence comprising at least 25, 50, 75, 100, 125, 150, 175, or 200 contiguous amino acids of SEQ ID NO:20. Typically the nucleic acid molecule encodes a polypeptide sequence of at least 70%, 75% or 80% identity to an amino acid sequence comprising the amino acid sequence of SEQ ID NO:20 or an amino acid sequence comprising at least 25, 50, 75, 100, 125, 150, 175, or 200 contiguous amino acids of SEQ ID NO:20. In further aspects, the encoded polypeptide is 85%, 90% or 95% identical to an amino acid sequence comprising the amino acid sequence of SEQ ID NO:20 or an amino acid sequence comprising at least 25, 50, 75, 100, 125, 150, 175, or 200 contiguous amino acids of SEQ ID NO:20 or 98%, 99%, 99.9% identical to an amino acid sequence comprising the amino acid sequence of SEQ ID NO:20 or an amino acid sequence comprising at least 25, 50, 75, 100, 125, 150, 175, or 200 contiguous amino acids of SEQ ID NO:20.

Provided herein is an isolated nucleic acid molecule having a nucleic acid sequence comprising or consisting of a wild-type P. pastoris MET19 gene sequence (SEQ ID NO:21), and homologs, variants and derivatives thereof. Further provided is a nucleic acid molecule comprising or consisting of a sequence which is a degenerate variant of the wild-type P. pastoris MET19 gene. In particular aspects, the nucleic acid molecule comprises or consists of a sequence which is a variant of the P. pastoris MET19 gene (SEQ ID NO: 21) having at least 65% identity to the wild-type gene or to a nucleotide sequence comprising at least 25, 50, 75, 100, 125, 150, 175, or 200 contiguous nucleotides of SEQ ID NO:21. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:21. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:21. The nucleic acid molecule encodes a polypeptide having the amino acid sequence of SEQ ID NO:22. Also provided is a nucleic acid molecule encoding a polypeptide sequence that is at least 65% identical to an amino acid sequence comprising the amino acid sequence of SEQ ID NO:22 or an amino acid sequence comprising at least 25, 50, 75, 100, 125, 150, 175, or 200 contiguous amino acids of SEQ ID NO:22. Typically the nucleic acid molecule encodes a polypeptide sequence of at least 70%, 75% or 80% identity to an amino acid sequence comprising the amino acid sequence of SEQ ID NO:22 or an amino acid sequence comprising at least 25, 50, 75, 100, 125, 150, 175, or 200 contiguous amino acids of SEQ ID NO:22. In further aspects, the encoded polypeptide is 85%, 90% or 95% identical to an amino acid sequence comprising the amino acid sequence of SEQ ID NO:22 or an amino acid sequence comprising at least 25, 50, 75, 100, 125, 150, 175, or 200 contiguous amino acids of SEQ ID NO:22 or 98%, 99%, 99.9% identical to an amino acid sequence comprising the amino acid sequence of SEQ ID NO:22 or an amino acid sequence comprising at least 25, 50, 75, 100, 125, 150, 175, or 200 contiguous amino acids of SEQ ID NO:22.

Provided herein is an isolated nucleic acid molecule having a nucleic acid sequence comprising or consisting of a wild-type P. pastoris MET22 gene sequence (SEQ ID NO:23), and homologs, variants and derivatives thereof. Further provided is a nucleic acid molecule comprising or consisting of a sequence which is a degenerate variant of the wild-type P. pastoris MET22 gene. In particular aspects, the nucleic acid molecule comprises or consists of a sequence which is a variant of the P. pastoris MET22 gene (SEQ ID NO: 23) having at least 65% identity to the wild-type gene or to a nucleotide sequence comprising at least 25, 50, 75, 100, 125, 150, 175, or 200 contiguous nucleotides of SEQ ID NO:23. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:23. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:23. The nucleic acid molecule encodes a polypeptide having the amino acid sequence of SEQ ID NO:24. Also provided is a nucleic acid molecule encoding a polypeptide sequence that is at least 65% identical to an amino acid sequence comprising the amino acid sequence of SEQ ID NO:24 or an amino acid sequence comprising at least 25, 50, 75, 100, 125, 150, 175, or 200 contiguous amino acids of SEQ ID NO:24. Typically the nucleic acid molecule encodes a polypeptide sequence of at least 70%, 75% or 80% identity to an amino acid sequence comprising the amino acid sequence of SEQ ID NO:24 or an amino acid sequence comprising at least 25, 50, 75, 100, 125, 150, 175, or 200 contiguous amino acids of SEQ ID NO:24. In further aspects, the encoded polypeptide is 85%, 90% or 95% identical to an amino acid sequence comprising the amino acid sequence of SEQ ID NO:24 or an amino acid sequence comprising at least 25, 50, 75, 100, 125, 150, 175, or 200 contiguous amino acids of SEQ ID NO:24 or 98%, 99%, 99.9% identical to an amino acid sequence comprising the amino acid sequence of SEQ ID NO:24 or an amino acid sequence comprising at least 25, 50, 75, 100, 125, 150, 175, or 200 contiguous amino acids of SEQ ID NO:24.

Provided herein is an isolated nucleic acid molecule having a nucleic acid sequence comprising or consisting of a wild-type P. pastoris MET27 gene sequence (SEQ ID NO:25), and homologs, variants and derivatives thereof. Further provided is a nucleic acid molecule comprising or consisting of a sequence which is a degenerate variant of the wild-type P. pastoris MET27 gene. In particular aspects, the nucleic acid molecule comprises or consists of a sequence which is a variant of the P. pastoris MET27 gene (SEQ ID NO: 25) having at least 65% identity to the wild-type gene or to a nucleotide sequence comprising at least 25, 50, 75, 100, 125, 150, 175, or 200 contiguous nucleotides of SEQ ID NO:25. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:25. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:25. The nucleic acid molecule encodes a polypeptide having the amino acid sequence of SEQ ID NO:26. Also provided is a nucleic acid molecule encoding a polypeptide sequence that is at least 65% identical to an amino acid sequence comprising the amino acid sequence of SEQ ID NO:26 or an amino acid sequence comprising at least 25, 50, 75, 100, 125, 150, 175, or 200 contiguous amino acids of SEQ ID NO:26. Typically the nucleic acid molecule encodes a polypeptide sequence of at least 70%, 75% or 80% identity to an amino acid sequence comprising the amino acid sequence of SEQ ID NO:26 or an amino acid sequence comprising at least 25, 50, 75, 100, 125, 150, 175, or 200 contiguous amino acids of SEQ ID NO:26. In further aspects, the encoded polypeptide is 85%, 90% or 95% identical to an amino acid sequence comprising the amino acid sequence of SEQ ID NO:26 or an amino acid sequence comprising at least 25, 50, 75, 100, 125, 150, 175, or 200 contiguous amino acids of SEQ ID NO:26 or 98%, 99%, 99.9% identical to an amino acid sequence comprising the amino acid sequence of SEQ ID NO:26 or an amino acid sequence comprising at least 25, 50, 75, 100, 125, 150, 175, or 200 contiguous amino acids of SEQ ID NO:26.

Provided herein is an isolated nucleic acid molecule having a nucleic acid sequence comprising or consisting of a wild-type P. pastoris MET28 gene sequence (SEQ ID NO:27), and homologs, variants and derivatives thereof. Further provided is a nucleic acid molecule comprising or consisting of a sequence which is a degenerate variant of the wild-type P. pastoris MET28 gene. In particular aspects, the nucleic acid molecule comprises or consists of a sequence which is a variant of the P. pastoris MET28 gene (SEQ ID NO: 27) having at least 65% identity to the wild-type gene or to a nucleotide sequence comprising at least 25, 50, 75, 100, 125, 150, 175, or 200 contiguous nucleotides of SEQ ID NO:27. The nucleic acid sequence can preferably have at least 70%, 75% or 80% identity to the wild-type gene or to a nucleotide sequence comprising at least 25, 50, 75, 100, 125, 150, 175, or 200 contiguous nucleotides of SEQ ID NO:27. Even more preferably, the nucleic acid sequence can have 85%, 90%, 95%, 98%, 99.9% or even higher identity to the wild-type gene or to a nucleotide sequence comprising at least 25, 50, 75, 100, 125, 150, 175, or 200 contiguous nucleotides of SEQ ID NO:27. The nucleic acid molecule encodes a polypeptide having the amino acid sequence of SEQ ID NO:28. Also provided is a nucleic acid molecule encoding a polypeptide sequence that is at least 65% identical to an amino acid sequence comprising the amino acid sequence of SEQ ID NO:28 or an amino acid sequence comprising at least 25, 50, 75, 100, 125, 150, 175, or 200 contiguous amino acids of SEQ ID NO:28. Typically the nucleic acid molecule encodes a polypeptide sequence of at least 70%, 75% or 80% identity to an amino acid sequence comprising the amino acid sequence of SEQ ID NO:28 or an amino acid sequence comprising at least 25, 50, 75, 100, 125, 150, 175, or 200 contiguous amino acids of SEQ ID NO:286. In further aspects, the encoded polypeptide is 85%, 90% or 95% identical to an amino acid sequence comprising the amino acid sequence of SEQ ID NO:28 or an amino acid sequence comprising at least 25, 50, 75, 100, 125, 150, 175, or 200 contiguous amino acids of SEQ ID NO:28 or 98%, 99%, 99.9% identical to an amino acid sequence comprising the amino acid sequence of SEQ ID NO:28 or an amino acid sequence comprising at least 25, 50, 75, 100, 125, 150, 175, or 200 contiguous amino acids of SEQ ID NO:28.

Provided herein are isolated polypeptides (including muteins, allelic variants, fragments, derivatives, and analogs) encoded by the nucleic acid molecules disclosed herein. In one embodiment, the isolated polypeptide comprises the polypeptide sequence corresponding to SEQ ID NO: 2, 4, 6, 8, 10, 12, 14, 16, 18, 20, 22, 24, 26, or 28. In particular aspects, the polypeptide comprises a polypeptide sequence at least 65% identical to an amino acid sequence comprising the amino acid sequence of SEQ ID NO: 2, 4, 6, 8, 10, 12, 14, 16, 18, 20, 22, 24, 26, or 28 or an amino acid sequence comprising at least 25, 50, 75, 100, 125, 150, 175, or 200 contiguous amino acids of SEQ ID NO: 2, 4, 6, 8, 10, 12, 14, 16, 18, 20, 22, 24, 26, or 28. In other aspects, the polypeptide has at least 70%, 75% or 80% identity to an amino acid sequence comprising the amino acid sequence of SEQ ID NO: 2, 4, 6, 8, 10, 12, 14, 16, 18, 20, 22, 24, 26, or 28 or an amino acid sequence comprising at least 25, 50, 75, 100, 125, 150, 175, or 200 contiguous amino acids of SEQ ID NO: 2, 4, 6, 8, 10, 12, 14, 16, 18, 20, 22, 24, 26, or 28. In further aspects, the identity is 85%, 90% or 95% and in further still aspects, the identity is 98%, 99%, 99.9% or even higher to an amino acid sequence comprising the amino acid sequence of SEQ ID NO: 2, 4, 6, 8, 10, 12, 14, 16, 18, 20, 22, 24, 26, or 28 or an amino acid sequence comprising at least 25, 50, 75, 100, 125, 150, 175, or 200 contiguous amino acids of SEQ ID NO: 2, 4, 6, 8, 10, 12, 14, 16, 18, 20, 22, 24, 26, or 28.

In other aspects, the isolated polypeptides comprising a fragment of the above-described polypeptide sequences are provided. These fragments include at least 20 contiguous amino acids, more preferably at least 25, 30, 35, 40, 45, 50, 60, 70, 80, 90, 100, 125, 150, 175, 200, or even more contiguous amino acids.

The polypeptides also include fusions between the above-described polypeptide sequences and heterologous polypeptides. The heterologous sequences can, for example, include heterologous sequences designed to facilitate purification and/or visualization of recombinantly-expressed proteins. Other non-limiting examples of protein fusions include those that permit display of the encoded protein on the surface of a phage or a cell, fusions to intrinsically fluorescent proteins, such as green fluorescent protein (GFP), and fusions to the IgG Fc region.

Also provided are vectors, including expression and integration vectors, which comprise all or a portion of the above nucleic acid molecules, as described further herein. In a first aspect, the vectors comprise the isolated nucleic acid molecules described above. In n further aspect, the vectors include the open reading frame (ORF) encoding Met1p, Met3p, Met4p, Met6p, Met7p, Met8p, Met10p, Met14p, Met16p, Met17p, Met19p, Met22p, Met27p, or Met28p operably linked to one or more expression control sequences, for example, a promoter sequence at the 5′ end and a transcription termination sequence at the 3′ end.

The vectors may also include an element which ensures that they are stably maintained at a single copy in each cell (e.g., a centromere-like sequence such as “CEN”). Alternatively, the autonomously replicating vector may optionally comprise an element which enables the vector to be replicated to higher than one copy per host cell (e.g., an autonomously replicating sequence or “ARS”). Methods in Enzymology, Vol. 350: Guide to yeast genetics and molecular and cell biology, Part B., Guthrie and Fink (eds.), Academic Press (2002).

In a further aspect, the vectors are non-autonomously replicating, integrative vectors designed to function as gene disruption or replacement cassettes.

In one aspect, the integration vector for constructing an auxotrophic strain comprises a heterologous nucleic acid fragment flanked on the 5′ end with a nucleic acid sequence from the 5′ region of the locus and on the 3′ end with a nucleic acid sequence from the 3′ region of the locus. The integration vector is capable of integrating into the genome by double-crossover homologous recombination. In particular aspects, the heterologous nucleic acid fragments encode one or more heterologous peptides, proteins, and/or functional nucleic acid molecules of interest.

In another aspect, the integration vector for constructing an auxotrophic strain comprises a nucleic acid fragment of the locus in which a region of the locus comprising all or part of the open reading frame (ORF) encoding Met1p, Met3p, Met4p, Met6p, Met7p, Met8p, Met10p, Met14p, Met16p, Met17p, Met19p, Met22p, Met27p, or Met28p has been excised. Thus, the integration vector comprises the 5′ region of the locus and the 3′ region of the locus and lacks part or all of the ORF encoding the Met1p, Met3p, Met4p, Met6p, Met7p, Met8p, Met10p, Met14p, Met16p, Met17p, Met19p, Met22p, Met27p, or Met28p. The integration vector is capable of integrating into the genome by double-crossover homologous recombination. In further aspects, the integration vector further includes one or more nucleic acid fragments, each encoding one or more heterologous peptides, proteins, and/or functional nucleic acid molecules of interest.

In a further aspect, provided is an integration vector comprising the open reading frame (ORF) encoding a P. pastoris Met1p, Met3p, Met4p, Met6p, Met7p, Met8p, Met10p, Met14p, Met16p, Met17p, Met19p, Met22p, Met27p, or Met28p operably linked to a heterologous promoter and a heterologous transcription termination sequence. The integration vector can further include a nucleic acid molecule that targets a region of the host cell genome for integrating the integration vector thereinto that does not include the ORF and which can further include one or more nucleic acid molecules encoding one or more heterologous peptides, proteins, and/or functional nucleic acid molecules of interest. The integration vector comprising the ORF encoding the P. pastoris Met1p, Met3p, Met4p, Met6p, Met7p, Met8p, Met10p, Met14p, Met16p, Met17p, Met19p, Met22p, Met27p, or Met28p is useful for complementing the auxotrophy of a host cell auxotrophic for methionine as a result of a deletion or disruption of the MET1, MET3, MET4, MET6, MET7, MET8, MET10, MET14, MET16, MET17, MET19, MET22, MET27, or MET28 locus, respectively.

In another aspect, provided is an integration vector comprising the open reading frame encoding a P. pastoris Met1p, Met3p, Met4p, Met6p, Met7p, Met8p, Met10p, Met14p, Met16p, Met17p, Met19p, Met22p, Met27p, or Met28p and the flanking promoter sequence and transcription termination sequence. The integration vector can further include a nucleic acid molecule that targets a region of the host cell genome for integrating the integration vector thereinto that does not include the ORF and which can further include one or more nucleic acid molecules encoding one or more heterologous peptides, proteins, and/or functional nucleic acid molecules of interest. The integration vector comprising the ORF encoding the P. pastoris Met1p, Met3p, Met4p, Met6p, Met7p, Met8p, Met10p, Met14p, Met16p, Met17p, Met19p, Met22p, Met27p, or Met28p is useful for complementing the auxotrophy of a host cell auxotrophic for methionine as a result of a deletion or disruption of MET1, MET3, MET4, MET6, MET7, MET8, MET10, MET14, MET16, MET17, MET19, MET22, MET27, or MET28 locus, respectively.

In general, the host cell is Pichia pastoris; however, in particular aspects, other useful lower eukaryote host cells can be used such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporiumi lucknowense, Fusarium sp., Fusarium gramineum, Fusarium venenatum, or Neurospora crassa.

Host cells defective or deficient in Met1p, Met3p, Met4p, Met6p, Met7p, Met8p, Met10p, Met14p, Met16p, Met17p, Met19p, Met22p, Met27p, or Met28p activity either by genetic engineering as disclosed herein or by genetic selection are auxotrophic for methionine and can be used to integrate one or more nucleic acid molecules encoding one or more heterologous peptides, proteins, and/or functional nucleic acid molecules of interest into the host cell genome using nucleic acid molecules and/or methods disclosed herein. In the case of genetic engineering, the one or more nucleic acid molecules encoding one or more heterologous peptides, proteins, and/or functional nucleic acid molecules of interest are integrated so as to disrupt an endogenous gene of the host cell and thus render the host cell auxotrophic.

According to one embodiment, a method for the genetic integration of separate heterologous nucleic acid sequences into the genome of a host cell is provided. In one aspect of this embodiment, genes of the host cell are disrupted by homologous recombination using integrating vectors. The integrating vectors carry an auxotrophic marker flanked by targeting sequences for the gene to be disrupted along with the desired heterologous gene to be stably integrated. When integrating more than one heterologous nucleic acid sequence, the order in which these plasmids are integrated is important for the auxotrophic selection of the marker genes. In order for the host cell to metabolically require a specific marker gene provided by the plasmid, the specific gene has to have been disrupted by a preceding plasmid.

For example, a first recombinant host cell is constructed in which the MET1 gene has been disrupted or deleted by an integration vector that targets the MET1 locus. The first recombinant host cell is auxotrophic for methionine. The first recombinant host is then transformed with an integration vector that targets a site that does not encode an enzyme involved in the biosynthesis of methionine and which carries the gene or ORF encoding the Met1p to produce a second recombinant host that is prototrophic for methionine. The second recombinant host is then transformed with an integration vector that targets another locus encoding an enzyme in the methionine biosynthetic pathway such as the MET3 locus but not the MET1 locus to produce a third recombinant host that is auxotrophic for methionine. The third recombinant host is then transformed with an integration vector that targets a site that does not encode an enzyme involved in the biosynthesis of methionine and which carries the gene or ORF encoding the Met3p or other methionine pathway enzyme other than Met1p to produce a second recombinant host that is prototrophic for methionine. This process can be continued in the same manner using integration vectors targeting loci in the pathway not previously targeted.

According to another embodiment, a method for the genetic integration of a heterologous nucleic acid sequence into the genome of a host cell is provided. In one aspect of this embodiment, a host gene encoding Met1p, Met3p, Met4p, Met6p, Met7p, Met8p, Met10p, Met14p, Met16p, Met17p, Met19p, Met22p, Met27p, or Met28p activity is disrupted by the introduction of a disrupted, deleted or otherwise mutated nucleic acid sequence obtained from the P. pastoris MET1, MET3, MET4, MET6, MET7, MET8, MET10, MET14, MET16, MET17, MET19, MET22, MET27, or MET28. Accordingly, disrupted host cells having a point mutation, rearrangement, insertion or preferably a deletion of a part or at least all of the open reading frame the Met1p, Met3p, Met4p, Met6p, Met7p, Met8p, Met10p, Met14p, Met16p, Met17p, Met19p, Met22p, Met27p, or Met28p activity (including a “marked deletion”, in which a heterologous selectable nucleotide sequence has replaced all or part of the deleted MET1, MET3, MET4, MET6, MET7, MET8, MET10, MET14, MET16, MET17, MET19, MET22, MET27, or MET28 gene are provided. Host cells disrupted in the URA5 gene (U.S. Pat. No. 7,514,253) and consequently lacking in orotate-phosphoribosyl transferase activity serve as suitable hosts for further embodiments of the invention in which heterologous nucleic acid sequences may be introduced into the host cell genome by targeted integration.

In a further embodiment, the MET1, MET3, MET4, MET6, MET7, MET8, MET10, MET14, MET16, MET17, MET19, MET22, MET27, or MET28 genes are initially disrupted individually using a series of knockout vectors, which delete large parts of the open reading frames and replace them with a PpGAPDH promoter/ScCYC1 terminator expression cassette and utilize the previously described PpURA5-blaster (Nett and Gerngross, Yeast 20: 1279-1290 (2003)) as an auxotrophic marker cassette. By knocking out each gene individually, the utility of these knockouts could be assessed prior to attempting the serial integration of several knockout vectors.

In a further embodiment, the individual disruption of the MET1, MET3, MET4, MET6, MET7, MET8, MET10, MET1-4, MET16, MET17, MET19, MET22, MET27, or MET28 genes of the host cell with specific integrating plasmids is provided. In one aspect of this embodiment, either a ura5 auxotrophic strain or any prototrophic strain is transformed with a plasmid that disrupts an MET gene using the URA5-blaster selection marker in the ura5 strain or the hygromicin resistance gene as a selection marker in any prototrophic strain. A vector comprising the MET gene is then used as an auxotrophic marker in a second transformation for the disruption of a gene encoding an enzyme in another biosynthetic pathway. In the third transformation, a vector comprising the gene encoding an enzyme in another biosynthetic pathway is used as an auxotrophic marker for the disruption of a different MET gene. For the fourth, fifth, sixth, and seventh transformations, disruption is alternated between the MET and genes encoding enzymes in another biosynthetic pathway until all available MET and genes encoding enzymes in another biosynthetic pathway are exhausted. In another embodiment, the initial gene to be disrupted can be any of the MET or genes encoding an enzyme in another biosynthetic pathway, as long as the marker gene encodes a protein of a different amino acid synthesis pathway than that of the disrupted gene. Furthermore, this alternating method needs only to be carried for as many markers and gene disruptions required for any given desired strain. For each transformation, one or multiple heterologous genes can be integrated into the genome and expressed using the constitutively active GAPDH promoter (Waterham et al. Gene 186: 37-44 (1997)) or any expression cassette that can be cloned into the plasmids using the unique restriction sites. U.S. Pat. No. 7,479,389, which is incorporated herein in its entirety, illustrates this method using ARG1, ARG2, ARG3, HIS1, HIS2, HIS5, and HIS6 genes.

In a further embodiment, the vector is a non-autonomously replicating, integrative vector which is designed to function as a gene disruption or replacement cassette. An integrative vector of the invention comprises one or more regions containing “target gene sequences” (sequences which can undergo homologous recombination with sequences at a desired genomic site in the host cell) linked to one of the fourteen genes (MET1, MET3, MET4, MET6, MET7, MET8, MET10, MET14, MET16, MET17, MET19, MET22, MET27, or MET28) cloned in P. pastoris.

In a further embodiment, a host gene that encodes an undesirable activity, (e.g., an enzymatic activity) may be mutated (e.g., interrupted) by targeting a P. pastoris—Met1p, Met3p, Met4p, Met6p, Met7p, Met8p, Met10p, Met14p, Met16p, Met17p, Met19p, Met22p, Met27p, or Met28p-encoding replacement or disruption cassette into the host gene by homologous recombination. In a further embodiment, an undesired glycosylation enzyme activity (e.g., an initiating mannosyltransferase activity such as OCH1) is disrupted in the host cell to alter the glycosylation of polypeptides produced in the cell.

Methods for the Genetic Integration of Nucleic Acid Sequences: Introduction of a Sequence of Interest in Linkage with a Marker Sequence

The isolated nucleic acid molecules encoding P. pastoris Met1p, Met3p, Met4p, Met6p, Met7p, Met8p, Met10p, Met14p, Met16p, Met17p, Met19p, Met22p, Met27p, or Met28p may additionally include one or more nucleic acid molecules encoding one or more heterologous peptides, proteins, and/or functional nucleic acid molecules of interest. The nucleic acid molecules encoding the one or more heterologous peptides, proteins, and/or functional nucleic acid molecules of interest may each be linked to one or more expression control sequences, e.g., promoter and transcription termination sequences, so that expression of the nucleic acid molecule can be controlled.

In another aspect, a heterologous nucleic acid molecule encoding one or more heterologous peptides, proteins, and/or functional nucleic acid molecules of interest in a vector is introduced into a P. pastoris host cell lacking expression of Met1p, Met3p, Met4p, Met6p, Met7p, Met8p, Met10p, Met14p, Met16p, Met17p, Met19p, Met22p, Met27p, or Met28p (i.e., the host cell is met1, met3, met4, met6, met7, met8, met10, met14, met16, met17, met19, met22, met27, or met28, respectively) and is, therefore, auxotrophic for methionine. The vector further includes a nucleic acid molecule that depending on the activity that is lacking in the host cell, encodes the appropriate Met1p, Met3p, Met4p, Met6p, Met7p, Met8p, Met10p, Met14p, Met16p, Met17p, Met19p, Met22p, Met27p, or Met28p activity that can complement the lacking activity and thus render the host cell prototrophic for methionine. Upon transformation of the vector into competent met1, met3, met4, met6, met7, met8, met10, met14, met16, met17, met19, met22, met27, or met28 host cells, cells containing the appropriate Met1p, Met3p, Met4p, Met6p, Met7p, Met8p, Met10p, Met14p, Met16p, Met17p, Met19p, Met22p, Met27p, or Met28p activity that can complement the lacking activity may be selected based on the ability of the cells to grow in a medium that lacks supplemental methionine. The nucleic acid molecule encoding the appropriate Met1p, Met3p, Met4p, Met6p, Met7p, Met8p, Met10p, Met14p, Met16p, Met17p, Met19p, Met22p, Met27p, or Met28p activity that can complement the lacking activity may include the homologous promoter and transcription termination sequences normally associated with the open reading frame encoding the activity or may comprise the open reading frame encoding the activity operably linked to nucleic acid molecules comprising heterologous promoter and transcription termination sequences.

In one embodiment, the method comprises the step of introducing into a competent P. pastoris met1, met3, met4, met6, met7, met8, met10, met14, met16, met17, met19, met22, met27, or met28 host cell an autonomously replicating vector which is passed from mother to daughter cells during cell replication. The autonomously replicating vector comprises a heterologous nucleic acid molecule sequences of interest linked to a nucleic acid sequence encoding the particular Met protein that complements the particular mer host cell and optionally comprises an element which ensures that it is stably maintained at a single copy in each cell (e.g., a centromere-like sequence such as “CEN”). In another embodiment, the autonomously replicating vector may optionally comprise an element which enables the vector to be replicated to higher than one copy per host cell (e.g., an autonomously replicating sequence or “ARS”).

In a further embodiment, the vector is a non-autonomously replicating, integrative vector which is designed to function as a gene disruption or replacement cassette. In general, an integrative vector comprises one or more regions comprising “target gene sequences” (nucleotide sequences that can undergo homologous recombination with nucleotide sequences at a desired genomic location in the host cell) linked to a nucleotide sequence encoding a P. pastoris Met1p, Met3p, Met4p, Met6p, Met7p, Met8p, Met10p, Met14p, Met16p, Met17p, Met19p, Met22p, Met27p, or Met28p activity. The nucleotide sequence may be adjacent to the target gene sequences (e.g., a gene replacement cassette) or may be engineered to disrupt the target gene sequences (e.g., a gene disruption cassette). The presence of target gene sequences in the replacement or disruption cassettes targets integration of the cassette to specific genomic regions in the host by homologous recombination.

In a further embodiment, a host gene that encodes an undesirable activity, (e.g., an enzymatic activity) may be mutated (e.g., interrupted) by targeting a P. pastoris Met1p, Met3p, Met4p, Met6p, Met7p, Met8p, Met10p, Met14p, Met16p, Met17p, Met19p, Met22p, Met27p, or Met28p activity-encoding replacement or disruption cassette into the host gene by homologous recombination. In a further embodiment, a gene encoding for an undesired glycosylation enzyme activity (e.g., an initiating mannosyltransferase activity such as Och1p) is disrupted in the host cell to alter the glycosylation of polypeptides produced in the cell.

In yet a further embodiment, a gene encoding a heterologous protein is engineered with linkage to a P. pastoris MET1, MET3, MET4, MET6, MET7, MET8, MET10, MET14, MET16, MET17, MET19, MET22, MET27, or MET28 gene within the gene replacement or disruption cassette. In a further embodiment, the cassette is integrated into a locus of the host genome which encodes an undesirable activity, such as an enzymatic activity. For example, in one preferred embodiment, the cassette is integrated into a host gene which encodes an initiating mannosyltransferase activity such as the OCH1 gene.

In a further embodiment, the method comprises the step of introducing into a competent met1, met3, met4, met6, met7, met8, met10, met14, met16, met17, met19, met22, met27, or met28 mutant host cell an autonomously replicating vector which is passed from mother to daughter cells during cell replication. The autonomously replicating vector comprises the appropriate P. pastoris gene that complements the mutation to render the host cell prototrophic for methionine, for example, the MET1, MET3, MET4, MET6, MET7, MET8, MET10, MET14, MET16, MET17, MET19, MET22, MET27, or MET28 gene, respectively.

The vectors disclosed herein are also useful for “knocking-in” genes encoding such glycosylation enzymes and other sequences of interest in strains of yeast cells to produce glycoproteins with human-like glycosylations and other useful proteins of interest. In a more preferred embodiment, the cassette further comprises one or more genes encoding desirable glycosylation enzymes, including but not limited to mannosidases, N-acetylglucosaminyltransferases (GnTs), UDP-N-acetylglucosamine transporters, galactosyltransferases (GalTs), sialytransferases (STs) and protein-mannosyltransferases (PMTS). U.S. Pat. No. 7,029,872, U.S. Pat. No. 7,449,308, U.S. Pat. No. 7,625,756, U.S. Pat. No. 7,198,921, U.S. Pat. No. 7,259,007, U.S. Pat. No. 7,465,577 and U.S. Pat. No. 7,713,719, U.S. Pat. No. 7,598,055, U.S. Published Patent Application No. 2005/0170452, U.S. Published Patent Application No. 2006/0040353, U.S. Published Patent Application No. 2006/0286637, U.S. Published Patent Application No. 2005/0260729, U.S. Published Patent Application No. 2007/0037248, Published International Application No. WO 2009105357, and WO2010019487, The disclosures of each incorporated by reference in their entirety.

Promoters are DNA sequence elements for controlling gene expression. In particular, promoters specify transcription initiation sites and can include a TATA box and upstream promoter elements. The promoters selected are those which would be expected to be operable in the particular host system selected. For example, yeast promoters are used when a yeast such as Saccharomyces cerevisiae, Kluyveromyces lactis, Ogataea minuta, or Pichia pastoris is the host cell whereas fungal promoters would be used in host cells such as Aspergillus niger, Neurospora crassa, or Tricoderma reesei. Examples of yeast promoters include but are not limited to the GAPDH, AOX1, SEC4, HH1, PMA1, OCH1, GAL1, PGK, GAP, TPI, CYC1, ADH2, PHO5, CUP1, MFα1, FLD1, PMA1, PDI, TEF, RPL10, and GUT1 promoters. Romanos et al., Yeast 8: 423-488 (1992) provide a review of yeast promoters and expression vectors. Hartner et al., Nucl. Acid Res. 36: e76 (pub on-line 6 Jun. 2008) describes a library of promoters for fine-tuned expression of heterologous proteins in Pichia pastoris.

The promoters that are operably linked to the nucleic acid molecules disclosed herein can be constitutive promoters or inducible promoters. An inducible promoter, for example the AOX1 promoter, is a promoter that directs transcription at an increased or decreased rate upon binding of a transcription factor in response to an inducer. Transcription factors as used herein include any factor that can bind to a regulatory or control region of a promoter and thereby affect transcription. The RNA synthesis or the promoter binding ability of a transcription factor within the host cell can be controlled by exposing the host to an inducer or removing an inducer from the host cell medium. Accordingly, to regulate expression of an inducible promoter, an inducer is added or removed from the growth medium of the host cell. Such inducers can include sugars, phosphate, alcohol, metal ions, hormones, heat, cold and the like. For example, commonly used inducers in yeast are glucose, galactose, alcohol, and the like.

Transcription termination sequences that are selected are those that are operable in the particular host cell selected. For example, yeast transcription termination sequences are used in expression vectors when a yeast host cell such as Saccharomyces cerevisiae, Kluyveromyces lactis, or Pichia pastoris is the host cell whereas fungal transcription termination sequences would be used in host cells such as Aspergillus niger, Neurospora crassa, or Tricoderma reesei. Transcription termination sequences include but are not limited to the Saccharomyces cerevisiae CYC transcription termination sequence (ScCYC TT), the Pichia pastoris ALG3 transcription termination sequence (ALG3 TT), the Pichia pastoris ALG6 transcription termination sequence (ALG6 TT), the Pichia pastoris ALG12 transcription termination sequence (ALG12 TT), the Pichia pastoris AOX1 transcription termination sequence (AOX1 TT), the Pichia pastoris OCH1 transcription termination sequence (OCH1 TT) and Pichia pastoris PMA1 transcription termination sequence (PMA1 TT). Other transcription termination sequences can be found in the examples and in the art.

Methods for integrating vectors into yeast are well known (See for example, U.S. Pat. No. 7,479,389, U.S. Pat. No. 7,514,253, U.S. Published Application No. 2009012400, and WO2009/085135; the disclosures of which are all incorporated herein by reference).

In particular embodiments, the vectors may further include one or more nucleic acid molecules encoding useful therapeutic proteins, e.g. including but not limited to Examples of therapeutic proteins or glycoproteins include but are not limited to erythropoietin (EPO); cytokines such as interferon α, interferon β, interferon γ, and interferon ω; and granulocyte-colony stimulating factor (GCSF); GM-CSF; coagulation factors such as factor VIII, factor IX, and human protein C; antithrombin III; thrombin; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; and urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4—Ig; transmembrane activator and calcium modulator and cyclophilin ligand; glucagon like protein 1; and IL-2 receptor agonist.

### Example 1

### General Materials and Methods

Escherichia coli strain DHSα (Invitrogen, Carlsbad, Calif.) was used for recombinant DNA work. P. pastoris strain YJN165 (ura5) (Nett and Gerngross, Yeast 20: 1279-1290 (2003)) was used for construction of yeast strains. PCR reactions were performed according to supplier recommendations using ExTaq (TaKaRa, Madison, Wis.), Taq Poly (Promega, Madison, Wis.) or Pfu Turbo® (Stratagene, Cedar Creek, Tex.). Restriction and modification enzymes were from New England Biolabs (Beverly, Mass.).

Yeast strains were grown in YPD (1% yeast extract, 2% peptone, 2% dextrose and 1.5% agar) or synthetic defined medium (1.4% yeast nitrogen base, 2% dextrose, 4×10⁻⁵% biotin and 1.5% agar) supplemented as appropriate. Plasmid transformations were performed using chemically competent cells according to the method of Hanahan (Hanahan et al., Methods Enzymol. 204: 63-113 (1991)). Yeast transformations were performed by electroporation according to a modified procedure described in the Pichia Expression Kit Manual (Invitrogen). In short, yeast cultures in logarithmic growth phase were washed twice in distilled water and once in 1M sorbitol. Between 5 and 50 μg of linearized DNA in 10 μl of TE was mixed with 100 μl yeast cells and electroporated using a BTX electroporation system (BTX, San Diego, Calif.). After addition of 1 ml recovery medium (1% yeast extract, 2% peptone, 2% dextrose, 4×10⁻⁵% biotin, 1M sorbitol, 0.4 mg/ml ampicillin, 0.136 mg/ml chloramphenicol), the cells were incubated without agitation for 4 h at room temperature and then spread onto appropriate media plates.

PCR analysis of the modified yeast strains was as follows. A 10 ml overnight yeast culture was washed once with water and resuspended 400 μl breaking buffer (100 mM NaCl, 10 mM Tris, pH 8.0, 1 mM EDTA, 1% SDS, 2% Triton X-100). After addition of 400 mg of acid washed glass beads and 400 μl phenol-chloroform, the mixture was vortexed for 3 minutes. Following addition of 200 μl TE (Tris/EDTA) and centrifugation in a microcentrifuge for 5 minutes at maximum speed, 500 μl of the supernatant was transferred to a fresh tube and the DNA was precipitated by addition of 1 ml ice-cold ethanol. The precipitated DNA was isolated by centrifugation, resuspended in 400 μl TE, with 1 mg RNase A, and the mixture was incubated for 10 minutes at 37° C. Then 1 μl of 4M NaCl, 20 μl of a 20% SDS solution and 10 μl of Qiagen Proteinase K solution was added and the mixture was incubated at 37° C. for 30 minutes. Following another phenol-chloroform extraction, the purified DNA was precipitated using sodium acetate and ethanol and washed twice with 70% ethanol. After air drying, the DNA was resuspended in 200 μl TE, and 200 ug was used per 50 μl PCR reaction.

BRIEF DESCRIPTION OF THE SEQUENCES

| SEQ | | |
|---|---|---|
| ID | | |
| NO: | Description | Sequence |
| 1 | MET1 | AATGATACCGTTCAAGACAAGCTCGTTGTCTTTTT |
| | | CAGCTCCCAAGAATGTTTTCCACAGGGCAAATAGC |
| | | TGAGATACCTCATCATCTGCGTCAACCTCCTCGTT |
| | | CAGCTCTACAGTAAGTTCAGAAGCATTTGCACTAG |
| | | AGCCAGACTCAGCAACGCCATCTTCATCTGTCTTT |
| | | TGCTTCTTCTTCTGTGCGGACTTTCCCAATCCAAG |
| | | CGGTCTTTTGGGTGGAGCCATTAGCTGATAATCAT |
| | | ACAGGAAAGTAAGAAAAAAGAAAGAAAGTTTTGAC |
| | | TTCAGCCTCGCCTCGGCTCGACTGTCTCCCCTATT |
| | | CTTGCATCTGCTTACATAAGTTGAAAAGTCGCTTG |
| | | GTAACATACGGAGGAGATATCAAGGTTCTCATCTA |
| | | TCTCGCATGCCATACAAATCACGTGCGATTGCATG |
| | | AAGCGATGAGTAGGCCTTTGAAAAAAAAAAAACAG |
| | | TTTCATAAGATTAGGTCTTCGTTATCCTCTATCCA |
| | | TACCCCCGACGATGGCCAAACTATTACTCGCAGAT |
| | | AACTGCCAAGGTCAAATCCATCTTGTGGTGGGCCT |
| | | AGAGCACCTGAATTTGTGTGTTTCAAGGGTGAAGA |
| | | CTATTCTGGAGGCTGGAGCCACACCGGTTCTAGTT |
| | | TCCCCACAAAAGTCCACGATGCTGGATTCTCTTCA |
| | | AGATCTAGCCACCCAGGGCACATTGAAGGTCGTAG |
| | | ATCAGACCTTCAGTATCTCACAGTTGACTCAATTG |
| | | GGGCGAGATGAAGTAGATAATGTGGTAGACAAGGT |
| | | GTTTGTGGTCTTGGACTCGCAATACGCCCAATTGA |
| | | AAAAAGACATCTCGGCTCACTGTAGAAGGCTAAGA |
| | | ATTCCTGTTTCAGTGGTAGATTCTCCAGAATTATG |
| | | CAGTTTCACTCTGTTATCAACCTATTCCAATGCTG |
| | | ATTTTCAGCTGGGAGTGACAACTAATGGAAAAGGA |
| | | TGTAAATTAGCATCTCGTATCAAAAGAGAACTAGT |
| | | TAGCACTCTACCTTCAAATATTGACAAGGTTTGCG |
| | | AAAACATTGGTAACCTAAGACACAGGATTCAGCAA |
| | | GAGGATGACGATCAAGTGGAGGAGATTTACAATAG |
| | | GTTACAATTGCTAGGAGAAGATGAAGATGATGCTA |
| | | TTCAGACATCCAGACTCAACCAGTTGGTTGAGGAG |
| | | TTTAACATGACCAAAGAACAGAAAAAACTACAAAG |
| | | AACGCGCTGGTTGTCGCAGTTAGTAGAGTATTACC |
| | | CTCTAGGAAAACTGGCAGAAGTTTCTGTGGACGAC |
| | | TTAAGTGCTGCATATCATGAATCTAGTAACAACGT |
| | | TGAAATTGCTCAGAATGGAACTTTCGACCATGCGA |
| | | AGAAAGGTTCTATATCATTGGTAGGAGCAGGACCA |
| | | GGAGCTGTCTCACTACTAACCTTGGGAGCACTGTC |
| | | CGAAATATACTCTGCAGATCTAATTCTTGCGGACA |
| | | AACTAGTACCGACTCAAGTTTTGGACTTAATTCCT |
| | | AGGAGAACGGAAGTTTTTATTGCTAGAAAGTTTCC |
| | | AGGAAATGCTGAAGCCGCACAACAGGAACTATTAT |
| | | CCAAGGGTTTAGCAGCCTTAGATGCTGGGAAGAAA |
| | | GTAATTCGCTTGAAGCAAGGTGACCCATACATTTT |
| | | TGGAAGAGGTGGGGAGGAATACCTATTTTTCGAAT |
| | | CTCAAGGTTACAGACCATTAGTTTTACCAGGCATC |
| | | ACTTCAGCATTGGCAGCACCTGTTCTGTCTCAAAT |
| | | TCCTGCAACGCATCGTGATGTTGCAGATCAAGTTC |
| | | TAATCTGCACAGGAACTGGACGTAGAGGAGCACTT |
| | | CCAAATATTCCAGAATTTGTGAAATCCCGTACTTC |
| | | AGTATTCCTTATGGCATTGCATCGTATTGTGGAGC |
| | | TTCTCCCTGTCCTTTTTGAGAAGGGGTGGGATCCA |
| | | AAGGTTCCTGCAGCAATTGTTGAACGAGCATCCTG |
| | | TCCAGATCAAAGGGTTATTAGAACTACATTAGAAA |
| | | ACGTTGGTCGAGCAGTCCAAGAATTTGGTTCCAGG |
| | | CCTCCTGGGCTTCTTGTGGTAGGATATTCATGTGG |
| | | GATCATTGAAAAGTTAGAGAAGGAGTGGGAAGTGG |
| | | TGGAAGGTTGGGATGACATTGGAGGATCGACCATA |
| | | CTAGATACAGTGTCCAACCTTTCCAAATGACTATG |
| | | AAGATAGTGAACTGCATTTTATTTATTGTATATGT |
| | | ATTTTAGACGCATTAATAGAGAGCCAAAAAGTTAT |
| | | ATCACAAGTTGATCTGTAGTGTCAGGTTGATTCCA |
| | | TGAGGATCAAAGTGCCATCCACCCATCCTGGGTAA |
| | | TCATGCAAAAAATGAAAGATTGGACGAGTTGGGAA |
| | | TCGAACCCAAGACCTCTCCCATGCTAAGGGAGCGC |
| | | GCTACCAACTACGCCACACGCCCATTTTCTCTTCG |
| | | GTGAAGGCTTTAAAAGATTTTGACCTAATCACTAT |
| | | TCTTTCGGTTTTAATACTACCATAAAATGACAGTT |
| | | AACTACTGTGCAGATAGCTTCATACATACTTAGAC |
| | | ACCTTATTGATAAAAAAAAATGACACTAGGCGCCG |
| | | AGAACCTTATTTACTTCCTAATTACTATGATAATA |
| | | AGTTCAATCTATAATAACCTGTGCTTATGTAATCA |
| | | TTATCCGCGTGTTTCCTCCACCCATAATTCTTCAA |
| | | CTAGTTTTCTAACCAATTGATTGAGTTTGACCATG |
| | | TTCTCCAACTCAATTAG |
| 2 | MET1 | MAKLLLADNCQGQIHLVVGLEHLNLCVSRVKTILE |
| | protein | AGATPVLVSPQKSTMLDSLQDLATQGTLKVVDQTF |
| | | SISQLTQLGRDEVDNVVDKVFVVLDSQYAQLKKDI |
| | | SAHCRRLRIPVSVVDSPELCSFTLLSTYSNADFQL |
| | | GVTTNGKGCKLASRIKRELVSTLPSNIDKVCENIG |
| | | NLRHRIQQEDDDQVEEIYNRLQLLGEDEDDAIQTS |
| | | RLNQLVEEFNMTKEQKKLQRTRWLSQLVEYYPLGK |
| | | LAEVSVDDLSAAYHESSNNVEIAQNGTFDHAKKGS |
| | | ISLVGAGPGAVSLLTLGALSEIYSADLILADKLVP |
| | | TQVLDLIPRRTEVFIARKFPGNAEAAQQELLSKGL |
| | | AALDAGKKVIRLKQGDPYIFGRGGEEYLFFESQGY |
| | | RPLVLPGITSALAAPVLSQIPATHRDVADQVLICT |
| | | GTGRRGALPNIPEFVKSRTSVFLMALHRIVELLPV |
| | | LFEKGWDPKVPAAIVERASCPDQRVIRTTLENVGR |
| | | AVQEFGSRPPGLLVVGYSCGIIEKLEKEWEVVEGW |
| | | DDIGGSTILDTVSNLSK |
| 3 | MET3 | CGCAAGATAATGGTGGCGTTTCGTCGTCTCCCCAA |
| | | CTTGAAGAGTTATTCTGAGTTGCAACAAGTCTAAG |
| | | TAGTAAGTAATTAAACCATCATGATCCTATGATCG |
| | | TGATCATTCATTAAAGCACGGTGTGGCAATTATTG |
| | | CTAGGGAGATCGTCACTGTATGGTGGCAGAATTAT |
| | | CTCTACAAGATGTCTCAAAGTCCCCACAAAGCTTG |
| | | GACCCTCTCATCTGTAATGCATTTTCCTGTAACTC |
| | | CCCTTAGCCACACGTCAAGGGCTCTGAATCCGTTG |
| | | AAAAGCTGTGGCGTCTGCCACCTTTAACGTCTTCA |
| | | TGAGGGATGTGCACGTGATATTGTCTTTCCCTTCT |
| | | CTAAAGCTTCGAAAAAAACGCATCTCAATGCGAGA |
| | | AGCAGATCGATATATATAAAGAACTAGTCCATTGA |
| | | AAGATCTCTCAATTTCACTGGAAACCAACTCAGAA |
| | | AGAAATGCCTTCTCCTCACGGTGGTGTGCTACAAG |
| | | ACCTTATTAAGCGTGACGCTTCTATCAAGGAAGAT |
| | | TTGTTGAAGGAAGTCCCTCAGCTTCAAAGTATTGT |
| | | GCTAACTGGTAGACAACTCTGTGATTTAGAGTTAA |
| | | TCCTAAATGGAGGTTTCAGTCCTTTGACAGGATTT |
| | | CTGACCGAGAAGGATTATCGCTCCGTTGTTGACGA |
| | | TTTGAGACTCGCCAGTGGTGATGTTTGGTCTATTC |
| | | CAATCACCCTGGACGTCAGCAAGACCGAGGCTAGT |
| | | AAGTTCCGTGTCGGCGAAAGAGTGGTGTTGAGAGA |
| | | TCTTCGTAACGACAATGCTCTGAGTATTCTGACCA |
| | | TCGAGGATATATACGAACCTGATAAGAACGTTGAG |
| | | GCTAAGAAAGTCTTCCGCGGTGATCCAGAACACCC |
| | | AGCTGTCAAGTACCTCTTTGATGTTGCCGGTGATG |
| | | TGTATATTGGTGGCGCTTTGCAAGCTCTACAATTG |
| | | CCTACTCATTACGACTACACCGCCCTGAGAAAAAC |
| | | GCCAGCCCAATTGAGGTCTGAGTTTGAGAGCCGTA |
| | | ATTGGGACCGTGTTGTCGCTTTCCAAACCCGTAAC |
| | | CCAATGCACAGAGCACACCGTGAGTTGACAGTTCG |
| | | TGCCGCCAGAGCTAACTTGGCCAATGTCCTGATTC |
| | | ATCCAGTTGTTGGTCTGACGAAACCAGGTGACATT |
| | | GACCACCACACTCGTGTCAAAGTTTACCAAGAGAT |
| | | CATTAAGAAGTATCCAAACGGTATGGCTCAGTTGT |
| | | CCCTGTTGCCATTGGCTATGCGTATGGCTGGTGAC |
| | | CGTGAGGCTGTTTGGCATGCTATCATCCGTAAGAA |
| | | CTACGGTGCTTCACACTTCATTGTTGGACGTGATC |
| | | ACGCTGGACCCGGTAAGAACTCCGCTGGTGTTGAC |
| | | TTCTACGGACCTTATGATGCACAGGAATTGGTAGA |
| | | GAAATACAAAGATGAGTTGGACATCCAAGTTGTTC |
| | | CTTTCCGTATGGTTACTTATCTTCCAGATGAGGAT |
| | | CGTTACGCTCCAATTGACACAGTCAAGGAGGGTAC |
| | | CCGTACCCTAAACATTTCGGGAACTGAGCTGCGTA |
| | | AACGTCTCAGAGATGGTACCCACATTCCAGAATGG |
| | | TTCTCTTACCCAGAAGTCGTTAAGATTTTGAGAGA |
| | | ATCCAATCCACCTCGTCCAAAACAAGGTTTCACTT |
| | | TGTACTTGACCGGATTGCCAAACTCCGGAGTTGAC |
| | | GCCTTGTCCAACGCTTTAGTTGCTACATTCAATCA |
| | | ATTCGAAGGCGCCCGCCACATTACTCTGCTAGATG |
| | | GCAAGAACGTCAACGAATCCGCATTGCCATTTGTT |
| | | GCCCATGAGTTGACACGCTCTGGGGCTGGTGTCAT |
| | | CATTGCTGACCCTACCAAGGCTCCTTCCGCTGCTG |
| | | AGATTGATTCTATTCGCAAGGAAGTATCCAAGGCG |
| | | GGCTCCTTTATCGTGATTTCATTGACTACTCCTTT |
| | | GAATCAAGTCTCTCAGCATGATCGTAAAGGATACT |
| | | ACTCCACTTCTCGTAAAGATGTTGACAACTACGTT |
| | | TTCCCAGAAGATGCTGAGATCAAGATCGACTTGGC |
| | | CAAAGAAGGTGCCATCGTTGGTATCCAAAAGGTGG |
| | | TCTTGTATTTGGAAGAACAGGGGTTCTTCCAGTTC |
| | | TAGATAGTAGACTTTATAATGATAGATTGAGATTA |
| | | TGCGAATCTTTGAATCGAGGGGAATGGTAACATCT |
| | | GACATCTTCTATCTCACGTCTGACACGTCTTGTTT |
| | | CTCCTAGCGATCGATCACTCCTGTCGACCCTCTGC |
| | | CCCCGAAAGATTCGGTCAAAAAGCAAAGGCAAACT |
| | | ATCCTCACTATTTACATCGCAGTCCATTTTTTTAT |
| | | TCAAACAATTTGCTGATTAACGCAATTGCAAACGG |
| | | ACCAATCACACTCCGGCTCCCAGAATCTAGGCATC |
| | | TTTTCTACACTTAAAAACTGAAAAACTCCGTTCAC |
| | | GTGCATGGTCGTGTCCCTTGCAATTATTCCGTAGG |
| | | TATCTCTCCACTGGGAAACAAAACAATCCTATCCG |
| | | ACAAACAATCGTCAGAACCATTACCACCCGTTGAA |
| | | TCCTCTGCTGTTAACCCCTAATTTCGGTGCTCAAT |
| | | AGCTTTTTCAAATACTAAGTGATAACATACTCATT |
| | | ATTTGAAGTTTGATTTTAGTGAGAAACGAGACTAC |
| | | CCAAACATTTGAGCGCATTCAAATTTTTGCCATCT |
| | | GACAACCGAGAATTGAGAATTTGAGAACCATTCAA |
| | | CGATTACGTAA |
| 4 | MET3 | MPSPHGGVLQDLIKRDASIKEDLLKEVPQLQSIVL |
| | protein | TGRQLCDLELILNGGFSPLTGFLTEKDYRSVVDDL |
| | | RLASGDVWSIPITLDVSKTEASKFRVGERVVLRDL |
| | | RNDNALSILTIEDIYEPDKNVEAKKVFRGDPEHPA |
| | | VKYLFDVAGDVYIGGALQALQLPTHYDYTALRKTP |
| | | AQLRSEFESRNWDRVVAFQTRNPMHRAHRELTVRA |
| | | ARANLANVLIHPVVGLTKPGDIDHHTRVKVYQEII |
| | | KKYPNGMAQLSLLPLAMRMAGDREAVWHAIIRKNY |
| | | GASHFIVGRDHAGPGKNSAGVDFYGPYDAQELVEK |
| | | YKDELDIQVVPFRMVTYLPDEDRYAPIDTVKEGTR |
| | | TLNISGTELRKRLRDGTHIPEWFSYPEVVKILRES |
| | | NPPRPKQGFTLYLTGLPNSGVDALSNALVATFNQF |
| | | EGARHITLLDGKNVNESALPFVAHELTRSGAGVII |
| | | ADPTKAPSAAEIDSIRKEVSKAGSFIVISLTTPLN |
| | | QVSQHDRKGYYSTSRKDVDNYVFPEDAEIKIDLAK |
| | | EGAIVGIQKVVLYLEEQGFFQF |
| 5 | MET4 | TGGTGAACCAAGAGGCGATTCCATCTACCAGAGGC |
| | | TGTTCTGGACCTGGCACCACAAGATCAACATTGTT |
| | | CTCCTGAGCGAACTGGACTAGTTGTGGGAAATTCT |
| | | CCTTGGAAGAGCCGATATTGACATTGGTAACTTTG |
| | | TCAAGTTTATGGGTACCACCGTTTCCAGGAGCGAC |
| | | ATAAACTTTGGCAACCTTGGGGGATTGAATGAGTT |
| | | TCCAGACCAGAGCATTCTCTCTGCCTCCGTTACCA |
| | | ACAACCAGAATGGTAGACATTTTGCGTTTAAGATA |
| | | GGATTTGGGTAGTTTAGGCGATGATTAATTGCAAA |
| | | GGGAAATTTTTTTTTTTTCATTTTTCCTTCTACGA |
| | | ATCTGGGGGAGAAGGTGGTGGGAGGATGCAGGTTG |
| | | TAGAAGGGAACTCCTGGTTTCCTGGAAGGAAGGAG |
| | | CGTAGCGCGGCGGGGTCAGACCGACTGACATGGCT |
| | | GCAGCAGTGCGATGCGAAAAAAAAAAATCTGAATA |
| | | AATGACACACCCAACGTCATCGTGAAAAGAAAAAC |
| | | AAATGTATTATGTAATCACTGAAACGTTTCTTCCA |
| | | ACGTCCGGTTAGACCCGAAAACTCGCAGATATCTG |
| | | TAAACATCTCCAAACCTCCTCAAAATCCAGTTGCC |
| | | GAAAAAAAAAACATGTCATGCCATATCACGTGAGA |
| | | TGGCGAAGCCACTGAAAAGAATTATCCTGCTTAGG |
| | | ATATGTCCCCCAGAATCTAGCAAAATTACTATTCC |
| | | CCCATAGTCTAGCCAAGACACAAAGTTGCTTAGCT |
| | | CTCAACACTTAAGCAACCACGTCCAGGACTCTACT |
| | | CGTCACAAAGGCCAATAGAAAGCCTCTAGAAGTAT |
| | | CTCAACATCACCTTCAAGTCCGGCTCAAATAGGTC |
| | | TTTTTAGTTTATTCAAAGTTTTTTTTCAAACCGTT |
| | | TGAGATTTTCTCCTTCCAAGAACTCAATTCCACAT |
| | | TCAACTTCCCTTGGTCTGTGGCTTCAACTCGAGAT |
| | | TCACCAGATATATTAGGAGCAGATCCACTACAATG |
| | | TCATTCAGCAGAGAGAACATGGTCGAAACAAATCT |
| | | CCTTAATGGAACCAGCCAGGATCAGGATAATACGG |
| | | AAACGTCAGCTGCTCTGTTGGAGCAGTTGGTCTAT |
| | | ATTGATCATCTGAACATTCCCGACGTCGACCCGAC |
| | | AAATTTCGATGATCAACTGTCTGCTGAGCTAGCAG |
| | | CTTTTGCCGACGACTCATTTATTTTCCCCGATGAA |
| | | GAGAAGCCGAAGAATAACGGCAATGATGAGCCAAA |
| | | TGATCCTGCTACTGTTTCCACGATCGGCACTAACA |
| | | CTCCTTCACCGTTGAACTTTCAGCGACAAGACCGT |
| | | GGCCATGGAAGACAAAAGTCTGGCACTGAATTATC |
| | | AGGTCTTCCGAAGGCGGTCGTTCCTCCTGGTGCTA |
| | | TGTCCTCTCTGGTAGCAGCTGGTCTGAATCAATCC |
| | | CAGATTGATACCTTGGCCACGTTGGTAGCGCAATA |
| | | CCAACATTTACCTCAACCACAGCAACAACGACAAC |
| | | AAGCAAACTACCTGCAATCAGTGAACCCAAATCTT |
| | | AATGAAAGAACCATCTTGAGCCTAAACGACGTATT |
| | | CAACTACAACTCTGGCTCGAGTAATCCTTCCAATA |
| | | GAGATGCGACCAGCACTACGAGCCCCATTTCACCT |
| | | TACGAGCAAATTCATGGGGTTCAGTCAAATGGTCA |
| | | GCAGCGTCGTGGTAATCAGACGGAGTCGGTTTCAT |
| | | CTCTCAGTTTTAACAATTCTGCTAGTGTAGAACCA |
| | | TCTTCTGTCCAGCAGGGACTTCGAAAGTCATCCAA |
| | | TGCGTCGTCGGCACAGGTGCCAGAGCATAAATATA |
| | | TGGCAGATGACGATAAGAGAAGAAGGAACACTGCA |
| | | GCCTCTGCCAGGTTCCGTATAAAAAAGAAGATGAA |
| | | AGAGCAAGCTATGGAGCGCAATATAAAGGAGCTGA |
| | | CGGAGAATGCTGAAAAGTTGGAACTAAAAATCCAA |
| | | AGGCTTGAAATGGAAAATAGATTATTACGCAACTT |
| | | GGTTGTGGAAAAAGGTGCCCAGAGGGACTCTCAAG |
| | | ATTTGGAGAGACTTCGTCGTAAGGCACAGCTGAAA |
| | | ACTGATAACTCCGAGTCCGGGGCTTCGAATTTGGA |
| | | ACCAGTGTTGAAGCAGGAACCAATATGAGTCTTAA |
| | | GGCGATGGGGTGAAATAGTCGTTCGTTTTTGTATA |
| | | CTACCCTTTGAAAGGGATTTATTGAATATTTAGTT |
| | | TAAGTCTGATGATTAGATGCTCAGTTTGTGCTACT |
| | | ATGGATCCAGGACGAGGTAGTAAGGAATGCTAGAG |
| | | ACTTGCCGGTCTTAGGAAGCCCATCCATGGGAGGG |
| | | AGCCGTCTACCACATATTATTTCTAGTGTCGTTCA |
| | | GGATCCCGGAAGTGGAACCTCTCTGAAAGAAGCGA |
| | | AAAAAAAACTAGAACTATTTCAACGCTCGTAAATT |
| | | AGACAATCGCTTGGAAGAGATAATGCCCATCAGTT |
| | | TATCATCCGTTGTTGGCTTTTGTAGGGTCCCCAAT |
| | | GGCGTCATTAAGGGTCTACCTCATGAGTCCCTCGT |
| | | AGCATCGACCTGGCCCTCTCGGCCCAGATGTTCCT |
| | | TGCAGTGTTCCGACATGCTTCAGGTTTTTTCGCGC |
| | | GAGCTTGTTTACACATCTCCTAAACAAGACATATC |
| | | AGACAGCATTCTCATTTGGTTCATAATATCCAACT |
| | | CAAACCATTGTTTCACCTCCGTCTATCAATCCTGA |
| | | CCCTGAGTCTTCTGGTCAC |
| 6 | MET4 | MSFSRENMVETNLLNGTSQDQDNTETSAALLEQLV |
| | protein | YIDHLNIPDVDPTNFDDQLSAELAAFADDSFIFPD |
| | | EEKPKNNGNDEPNDPATVSTIGTNTPSPLNFQRQD |
| | | RGHGRQKSGTELSGLPKAVVPPGAMSSLVAAGLNQ |
| | | SQIDTLATLVAQYQHLPQPQQQRQQANYLQSVNPN |
| | | LNERTILSLNDVFNYNSGSSNPSNRDATSTTSPIS |
| | | PYEQIHGVQSNGQQRRGNQTESVSSLSFNNSASVE |
| | | PSSVQQGLRKSSNASSAQVPEHKYMADDDKRRRNT |
| | | AASARFRIKKKMKEQAMERNIKELTENAEKLELKI |
| | | QRLEMENRLLRNLVVEKGAQRDSQDLERLRRKAQL |
| | | KTDNSESGASNLEPVLKQEPI |
| 7 | MET6 | ACGCATATTGAGACAGTAGCGACTCTGTCTTGTTC |
| | | TCCAATTGCAACGCTTGGGACCTTGTTTGGGAGTA |
| | | GTTCGACATTGGGTTCCTCTGAGATGTTTGACAAG |
| | | TGAGAGCTAAATGATAACGAAATGCCTACCTGGCA |
| | | GGACGTGTACTGATCAAACCTCCCAGGTTCACATC |
| | | GGTCACTTGCTCGATTCCAGCAAGCTACGCCCTTT |
| | | AAGTTTTGTCCACCAGCTTTGCGCACTCTCTTGCC |
| | | TCTTTCGAACCCCGAGCGCGCTTCAGATGCAGATC |
| | | AAAGCACGAGATGCCACGTGACAGTCCATGTATTC |
| | | TTTCGTTTATCTTCGTATAGACAATAATATTTCAT |
| | | TGACTCTGTCAATGGTCGATGTTCACGTGCAAAAA |
| | | TTTTCAATTCGTTTGTTGGGCGACACCTCCACTAC |
| | | GTATATAAAAGGATCCGACCGCCCACTTGTCCTTG |
| | | CTTCCTGTAATTGTTTCCCAAACAACTAGTAGTTC |
| | | AATTATTACTAAAATGGTTCAATCATCTGTCTTAG |
| | | GTTTCCCACGTATCGGTGCCTTTAGAGAATTAAAG |
| | | AAGACCACCGAGGCCTACTGGTCTGGTAAGGTCGG |
| | | AAAAGACGAGCTTTTCAAAGTCGGAAAGGAGATCA |
| | | GAGAGAACAACTGGAAGCTGCAAAAGGCTGCTGGT |
| | | GTCGATGTCATTGCTTCCAACGACTTCTCCTACTA |
| | | CGACCAAGTTCTTGACCTGTCTCTTCTGTTTAACG |
| | | CTATTCCAGAGAGATACACTAAGTACGAGTTGGAC |
| | | CCAATTGACACCCTATTCGCCATGGGTAGAGGTTT |
| | | ACAAAGAAAGGCCACCGACTCCGAGAAGGCTGTTG |
| | | ATGTCACCGCTTTGGAGATGGTTAAATGGTTTGAT |
| | | TCTAACTACCACTACGTCAGACCCACTTTCTCTCA |
| | | CTCCACTGAGTTCAAGCTGAATGGTCAAAAGCCAG |
| | | TTGACGAGTACTTAGAGGCCAAGAAACTTGGAATT |
| | | GAGACTAGACCAGTTGTTGTTGGTCCAGTTTCTTA |
| | | CCTGTTCTTGGGTAAGGCTGACAAAGACTCTCTTG |
| | | ACTTGGAGCCAATCTCTCTTTTGGAGAAGATTTTG |
| | | CCTGTCTACGCTGAACTACTGGCCAAGCTGTCCGC |
| | | TGCTGGTGCCACTTCCGTGCAAATCGATGAGCCAA |
| | | TCCTGGTTTTAGATCTCCCAGAGAAGGTTCAAGCT |
| | | GCTTTCAAGACTGCTTATGAATACCTTGCCAATGC |
| | | TAAGAACATTCCAAAGTTGGTTGTTGCCTCCTACT |
| | | TCGGTGATGTCAGACCAAACTTGGCTTCTATCAAG |
| | | GGTTTACCAGTCCACGGTTTCCACTTTGACTTTGT |
| | | CAGAGCTCCAGAGCAATTCGACGAAGTTGTTGCCG |
| | | CATTGACAGCTGAGCAAGTTTTGTCCGTCGGTATC |
| | | ATTGACGGTAGAAACATCTGGAAAGCTGATTTCTC |
| | | CGAGGCTGTTGCTTTCGTTGAAAAGGCTATTGCTG |
| | | CTTTGGGTAAGGACAGAGTTATTGTTGCCACCTCT |
| | | TCCTCTTTGTTGCACACACCAGTTGACTTGACCAA |
| | | CGAAAAGAAGCTGGACTCCGAGATCAAGAACTGGT |
| | | TTTCGTTTGCTACCCAAAAGTTGGATGAGGTTGTT |
| | | GTCGTCGCCAAGGCTGTATCTGGTGAGGATGTCAA |
| | | GGAGGCTTTGTCTGTAAATGCCGCTGCCATCAAGT |
| | | CTAGAAAGGACTCTGCTATCACTAACGATGCTGAT |
| | | GTTCAAAAGAAGGTTGACTCCATCAATGAGAAGTT |
| | | ATCTTCCAGAGCTGCTGCTTTCCCTGAAAGATTGG |
| | | CTGCTCAAAAGGGCAAGTTCAACTTGCCTTTGTTC |
| | | CCAACCACCACCATTGGTTCTTTCCCACAGACTAA |
| | | GGATATCAGAATCAACAGAAACAAGTTCACCAAGG |
| | | GTGAAATCACTGCTGAGCAATATGACACTTTCATC |
| | | AAATCTGAGATTGAGAAAGTCGTCAGATTCCAGGA |
| | | GGAGATTGGTTTGGATGTTCTTGTCCACGGTGAAC |
| | | CAGAGAGAAACGATATGGTTCAATACTTTGGTGAG |
| | | CAGCTGAAGGGTTTTGCCTTCACCACCAATGGTTG |
| | | GGTCCAATCTTACGGTTCTCGTTACGTTAGACCAC |
| | | CTGTGGTTGTCGGTGACGTTTCTAGACCTCATGCC |
| | | ATGTCTGTCAAGGAGTCTGTTTACGCTCAGTCCAT |
| | | CACTAAGAAGCCTATGAAGGGTATGTTGACTGGTC |
| | | CTATCACCGTCTTGAGATGGTCTTTCCCAAGAAAC |
| | | GACGTTTCCCAAAAGGTTCAAGCTCTGCAATTGGG |
| | | TCTTGCTCTGAGAGATGAAGTTAACGACTTAGAGG |
| | | CCGCAAGTGTCGAAGTTATTCAAGTTGACGAGCCA |
| | | GCTATTAGAGAAGGTTTGCCATTGAGAAGCGGTCA |
| | | AGAAAGATCTGACTACTTGAAATACGCTGCTGAAT |
| | | CTTTCAGAATTGCTACTTCCGGTGTCAAGAACACT |
| | | ACTCAGATCCACTCTCACTTCTGTTACTCTGATTT |
| | | GGATCCTAACCATATCAAGGCTTTGGACGCTGACG |
| | | TTGTCTCTATTGAGTTCTCTAAGAAAGATGATCCT |
| | | AACTACATTCAAGAGTTCTCTAACTACCCTAACCA |
| | | CATCGGATTGGGTTTGTTTGACATCCACTCTCCAA |
| | | GAATTCCTTCCAAGGAGGAGTTCATTGCCAGAATT |
| | | GGTGAGATTCTTAAGGTGTACCCAGCTGACAAGTT |
| | | CTGGGTCAACCCTGACTGTGGTTTGAAGACCAGAG |
| | | GCTGGGAGGAGGTCAGAGCCTCTTTGACTAATATG |
| | | GTTGAAGCTGCTAAGACCTACCGTGAAAAGTACGC |
| | | TCAGAATTAAGCCTGAATAAATTCTTTGCGTATTG |
| | | ATTACATGCTGCATTTATTCAACATTAATGTTTTG |
| | | CATATAATGATCATATTTGAATCATTATCATTTTG |
| | | TTCAATTACTTCTTTCTAGACGATCGTTTGTATTA |
| | | TGTGTTATAGGGGGGATTTCAACATCGGTTAATTA |
| | | AAGTTTATTACTACTTTTGTGATCTGTAGGAAAAT |
| | | TAGTCTTGTAGTGTAGAGTGGACAGGCAGACGCAG |
| | | GGAAGACTCACTTCACCAGTTCGAGAGCAGGAACG |
| | | GACCCACGATTCCTCCCAGCAAAACCGTGGGCCCT |
| | | TCAGATATCACTTCGCTAGATTTCTAGTGGCAACT |
| | | CCTTTTTGAACCCTATTAAA |
| 8 | MET6 | MVQSSVLGFPRIGAFRELKKTTEAYWSGKVGKDEL |
| | protein | FKVGKEIRENNWKLQKAAGVDVIASNDFSYYDQVL |
| | | DLSLLFNAIPERYTKYELDPIDTLFAMGRGLQRKA |
| | | TDSEKAVDVTALEMVKWFDSNYHYVRPTFSHSTEF |
| | | KLNGQKPVDEYLEAKKLGIETRPVVVGPVSYLFLG |
| | | KADKDSLDLEPISLLEKILPVYAELLAKLSAAGAT |
| | | SVQIDEPILVLDLPEKVQAAFKTAYEYLANAKNIP |
| | | KLVVASYFGDVRPNLASIKGLPVHGFHFDFVRAPE |
| | | QFDEVVAALTAEQVLSVGIIDGRNIWKADFSEAVA |
| | | FVEKAIAALGKDRVIVATSSSLLHTPVDLTNEKKL |
| | | DSEIKNWFSFATQKLDEVVVVAKAVSGEDVKEALS |
| | | VNAAAIKSRKDSAITNDADVQKKVDSINEKLSSRA |
| | | AAFPERLAAQKGKFNLPLFPTTTIGSFPQTKDIRI |
| | | NRNKFTKGEITAEQYDTFIKSEIEKVVRFQEEIGL |
| | | DVLVHGEPERNDMVQYFGEQLKGFAFTTNGWVQSY |
| | | GSRYVRPPVVVGDVSRPHAMSVKESVYAQSITKKP |
| | | MKGMLTGPITVLRWSFPRNDVSQKVQALQLGLALR |
| | | DEVNDLEAASVEVIQVDEPAIREGLPLRSGQERSD |
| | | YLKYAAESFRIATSGVKNTTQIHSHFCYSDLDPNH |
| | | IKALDADVVSIEFSKKDDPNYIQEFSNYPNHIGLG |
| | | LFDIHSPRIPSKEEFIARIGEILKVYPADKFWVNP |
| | | DCGLKTRGWEEVRASLTNMVEAAKTYREKYAQN |
| 9 | MET7 | TGACTTCATGGAGAACATTTCTTTGGCCGGTAAAA |
| | | CCAACTTCTTCGAAAAGAGAGTTTCTGATTACCAA |
| | | AAGGCAGGTGTCATGGCTTCTACAGACAAAACTTC |
| | | TAATGATGATGCCTTTGCCTTTGATGAGGATTTCT |
| | | AGATCTTTTTTGGTCAATAATAGGGGGGTTTTTTA |
| | | CAAAGGTTAGCGGTTAGAGACTTAACGTCATATTA |
| | | CGTTATAATGTATATTAAATTTAGTTATGATAATT |
| | | TTTCGTTATCTGGTAACTTTAGGCTTGGTTTCTGT |
| | | TATTCTTTTTTTTTCTTTTTTATTTATCCCTCACG |
| | | GACGGATAGATGCCCGAATTAAACAAGGAATTCTT |
| | | CATAGCGATCCCCTTTAAGCAGTTACTTCCCAGCG |
| | | CCCTCCTAGAGTCTTTTCTTGGTTGCCTGCACACT |
| | | ACCCAAAAACTTTAAAAACGTCAGGCCTGCCAGAG |
| | | ATTTTCCTCTCTTTGTTCGATCCAACCAGTATGGG |
| | | ACAGCCAGATATGCCATTACATCGTTCGTATAAAG |
| | | ATGCTATAAGGGCCTTGAACTCCCTTCAGTCCAAC |
| | | TACGCCACAATTGAGGCTATTCGAAAGTCTGGTAA |
| | | CAACAGAAGTGCTAATAACATCCCTGAAATGGTGG |
| | | AATGGACCAGAAGGATAGGTTACTCTCCAACCGAA |
| | | TTCAACAGGTTGAACATCATTCATGTGACGGGGAC |
| | | TAAAGGTAAGGGTTCCACATGTGCATTTGTGCAGT |
| | | CAATTTTGAAGAGATACAAGAACAAAGACTTCGCC |
| | | ACAGCGTCCAGAAACTCAAGTAGCTCCACCCTTGC |
| | | AAGTTCAAGATCCAATGAACTTGAAAAACCCCACA |
| | | TAACCAAGGTTGGATTATATTCCTCTCCACACTTG |
| | | AAGTCTGTGCGGGAACGTATCAGAATCAATGGGAA |
| | | GCCTCTAACTGAGGACCTTTTCACCAAATACTTCT |
| | | TTGAAGTATGGGACAGACTTGAAAACTCTGAATCT |
| | | AACCCTTCTACGTTCCCTCAGTTGAGCCCAGGTTT |
| | | GAAACCTGCCTACTTCAAATATTTAACCCTACTGT |
| | | CTTTCCATGTATTCATGAGTGAAAACGTCGATTCT |
| | | GCCATCTACGAAGTTGGAGTTGGTGGAGAGTTCGA |
| | | TTCCACGAACATAATAGAAAAACCCACAGTTACTG |
| | | GAGTTTCTGCTCTTGGCATTGATCACACTTTCATG |
| | | CTGGGAAATACCCTCACAGATATTGCCTGGAACAA |
| | | ATCTGGTATATTCAAAGAAGGAGTTCCAGCTGTTT |
| | | CAGTACCACAACCAGAGGAAGGTATGAATGAACTC |
| | | GTCAGAAGAGCTGAAGAGAGAAAGGTAAAGTTCTT |
| | | CAAAGTCGTTCCTGACAGGGATCTCAGTGATATCA |
| | | AACTGGGACTCGCAGGTGCTTTCCAGAAAGAGAAT |
| | | GCGAACTTGGCCATAGAGCTTGCCGCAATTCACCT |
| | | ACAGAAATTGGGATTCAAAGTTGATGTAAAGGATG |
| | | ACCTTCCAGATGAATTTGTGGAGGGTTTATCTAGC |
| | | GCAACGTGGCCTGGTAGATGTCAGATTATAGAAGA |
| | | ACCCGAGAACCAAATTACTTGGTATTTGGATGGTG |
| | | CCCATACCAAGGAAAGTATCGAGGCTTCTTCCCAG |
| | | TGGTTCACTGAAAAGCAAACCAAGTCTGATCAAAC |
| | | TGTACTTTTGTTTAATCAGCAAACTAGAGATGGTG |
| | | AAGCACTGATTAAACAGTTGCATGGCGTAGTGTAC |
| | | CCGAAATTAAAGTTCAACCATGTTATCTTCACTAC |
| | | TAACTTAACGTGGTCAGACGGATACTCTGATGACC |
| | | TCGTGTCTTTGAACATCTCCAAAGAGGAAATTGAT |
| | | AATATGGATGTTCAGAAGGCACTTGCTGAAACTTG |
| | | GAACAGTCTCGATAAAGCAAGTCGTAAACATATTT |
| | | TTCACGATATTGAAACATCCATTAACTTTATTCGT |
| | | TCGCTCGAAGGTTCTGTGGACGTTTTTGTTACCGG |
| | | ATCTTTACACTTGGTGGGAGGATTCCTGGTTGTTT |
| | | TGGATAGAAAAGATTTGCCTAATTAATTTATTGAC |
| | | TGCTTATTAAAAAAATCCCCTTTTCTTCCTGGACC |
| | | CATCTAATCTCTAATGTTGCAATAGATCCGGAATG |
| | | TCCAGCAATTCCTCTTCTTCGTCAATGTCCAGGAC |
| | | TTTGCTAACACCTGCCTTGTTTCGGAAAAGCTCTA |
| | | CTGCTCCTGCATACAACATTTTGCCCTCTTGAGTA |
| | | GACGTTTGGGGCCTGAAGTACACCAGGACCAGGGG |
| | | TGAAGATTTTCTTCCATCTTGCAGTGTTATTGGAT |
| | | ATGACAACAGTATAAATCTTGGCGAACTATCAGGA |
| | | ACTTCATCTACCAAGTCCTCTAAAGAGGTAATGAC |
| | | ATCAGTTTCAGCCTTGATTTCGT |
| 10 | MET7 | MGQPDMPLHRSYKDAIRALNSLQSNYATIEAIRKS |
| | protein | GNNRSANNIPEMVEWTRRIGYSPTEFNRLNIIHVT |
| | | GTKGKGSTCAFVQSILKRYKNKDFATASRNSSSST |
| | | LASSRSNELEKPHITKVGLYSSPHLKSVRERIRIN |
| | | GKPLTEDLFTKYFFEVWDRLENSESNPSTFPQLSP |
| | | GLKPAYFKYLTLLSFHVFMSENVDSAIYEVGVGGE |
| | | FDSTNIIEKPTVTGVSALGIDHTFMLGNTLTDIAW |
| | | NKSGIFKEGVPAVSVPQPEEGMNELVRRAEERKVK |
| | | FFKVVPDRDLSDIKLGLAGAFQKENANLAIELAAI |
| | | HLQKLGFKVDVKDDLPDEFVEGLSSATWPGRCQII |
| | | EEPENQITWYLDGAHTKESIEASSQWFTEKQTKSD |
| | | QTVLLFNQQTRDGEALIKQLHGVVYPKLKFNHVIF |
| | | TTNLTWSDGYSDDLVSLNISKEEIDNMDVQKALAE |
| | | TWNSLDKASRKHIFHDIETSINFIRSLEGSVDVFV |
| | | TGSLHLVGGFLVVLDRKDLPN |
| 11 | MET8 | AAGGAAGGGAAGTAGATAATAACAAATAGCAATCA |
| | | GAGCTTAGCCTTGGGTGGCAAACTTGCTTTCAGTG |
| | | GCAAAACAGTTTTTTTCCTGGAAGAGTCTTCTTCT |
| | | TTGCCGACTATCATTGCTTGCCATTGCACATCCAT |
| | | ATTGTAGTTCTTCGACCTTGGACTATGGTGAGAAG |
| | | AGGAGTTAAAAGTAGCAACATCCAAGTTTTATCGC |
| | | GATTAGTTATCCGGGTAACCCATAAGGCAGCTTGC |
| | | CACGTCGCCATCAAATTGGATGAATTGGGGCTGTA |
| | | CTGCGGGCTTAGACCAGATGGTTGAGCGACATGGG |
| | | AGAACACGGATAAGTCCATTCCAATGCGTATTATT |
| | | GGAAGAATACTTTACCCAGACAGACATTACTAGGA |
| | | GAATACGTAGCTAATCTAGGACAAGTGATTGGTAA |
| | | GCAGAGAAAAAAACAATCAATCGCGTTCTGATATT |
| | | TACCATGTCACGAATTGGAAGGCAAAATATCGTTA |
| | | CCCGGATAACAGCTGAGCATCACTCACAACACTTC |
| | | GTGTGTTGCAAGAGTATAATTAGTCCAAAACGAGT |
| | | AACTACACGTAAGAACGGATGTATTTGAGTGATAC |
| | | ATACTAAGTACAACCTCCACGTTAATTACTCAAAT |
| | | TATATTGAGTGATGGACCCCCGAATTTTCCGCAGT |
| | | GATTGAAATGTTTCAACTGAAAGTCCGCATTGACT |
| | | AACAACTCTGGGTGTGAAGTGATCACCGATAAAGT |
| | | TACATCCCTTCCTTACCGACAGCTCGTTTCTCACA |
| | | CTCCGTCTGTTTCTTGCAATCCAAGCTGAATTCTT |
| | | CGACCAATTTAGGGATTTCAGAGGTGTCAACTTAT |
| | | ATATTCATTCTCTTTTTCACCATCAGCGTGCTCCA |
| | | TCTTATCATCACATTTAACTGCGCGAAAGATTCCA |
| | | TTAACCCCAGGCGGATTAAAATGCCATTAACACCA |
| | | GTTTTGGAACTAATCCATCATGTCAATCGAAATCC |
| | | CAGAGCCCAACGGTTCTTTGATGTTGGCTTGGCAA |
| | | GTAAGAAATCGTCATGTACTTCTTGTGGGTGGAGG |
| | | AGCAGTTGCCCTTTCTCGAATTGAACTACTTCTTC |
| | | AAGCCGATGCAAAAGTTACAGTGGTTGCTCCCAAG |
| | | ATAGATCCTACCATTGAACAGTATGAAAAATTGGG |
| | | GTTATTATACAAAGTTCATAGAAGAAAGTTCCTCA |
| | | AAGATGATTTGAAAATGTATGAAGGTGAAGCGTCC |
| | | AGAAAGCTGGACCAATTTTCTGGTGTAGACCATTT |
| | | TGGGCCCGAAGAGATGGAGCAAATAGAACAGGCAG |
| | | TTAAGCAGGAACAATTTGCATTGGTTCTAACCGCA |
| | | ATAGATGATAAAAATCTTTCCAAGCAAATATACTA |
| | | TTGGTGTAAAGCTGGGCGAATGCAAGTAAACATCG |
| | | CCGACAAACCCAAACAATGTGATTTCTACTTTGGG |
| | | TCAGTAGTAAGACAGGGGAGTATACAAATTATGAT |
| | | TAGTTCAAACGGAAAGTCTCCAAGATTGTGTCATA |
| | | AACTTAAGCACGATAAGCTGGAACCTCTACTTGCC |
| | | AGCTTGGATGCAAAAACTGCAGTGGACAATTTGGG |
| | | GAAAATGCGTGGAGAATTAAGGCATAGGGTAGCTC |
| | | CAGGAGAGGATACTCCCACCATCAAAGAACGAATG |
| | | GCTTGGAACACTCAGGTGACTGACCTGTTTACAAT |
| | | TGAAGAATGGGGCCAATTTGACGACACAGCACTGA |
| | | ATAGGCTTCTGAGTTTTTACCCCAAAGTACCTCAA |
| | | CGTCAGGACATAATAGTCGTTCCGCTAGAGAACTT |
| | | TTAGGTTACGTAGTAATACATGTGATAACAGCATC |
| | | TCGGTCATTGATAGATTCAAGGAGATACGGTAGGA |
| | | GAAGCCAGTTCTGGAGAATTAGCACCTGATAAATT |
| | | CGTGTTCGGGGAACTAGGAGGAGCTGGTTCCTTGG |
| | | CTGATAATATTGGACTAGTTACTGTTTCTTCAAAG |
| | | TCTTCCAAAGACTTCGAAGGGGAGCTAGTCGTAGC |
| | | AGAAGAAGACGCTGGTACTTCCTTAGATGTGGCCC |
| | | CCATCGAACCGTTACCACTGATGTTGGGGGCTCCA |
| | | ATAGAACTTCCCACTGGACTTTGAACCATATAGGG |
| | | GCCCGAATACTGTCCCGGATCCATCTCACTATAAA |
| | | C |
| 12 | MET8 | MSIEIPEPNGSLMLAWQVRNRHVLLVGGGAVALSR |
| | protein | IELLLQADAKVTVVAPKIDPTIEQYEKLGLLYKVH |
| | | RRKFLKDDLKMYEGEASRKLDQFSGVDHFGPEEME |
| | | QIEQAVKQEQFALVLTAIDDKNLSKQIYYWCKAGR |
| | | MQVNIADKPKQCDFYFGSVVRQGSIQIMISSNGKS |
| | | PRLCHKLKHDKLEPLLASLDAKTAVDNLGKMRGEL |
| | | RHRVAPGEDTPTIKERMAWNTQVTDLFTIEEWGQF |
| | | DDTALNRLLSFYPKVPQRQDIIVVPLENF |
| 13 | MET10 | ACATTTCCCAAATGGGGTAGAAAGAGCTTAGCTTC |
| | | GGTCGTTACTTCGTTGGACGCTGACGGTATTGACC |
| | | TTTTAGAGCGCTTGCTTGTCTACGACCCGGCCGGC |
| | | CGAATCTCCGCCAAGCGTGCTCTTCAGCACTCCTA |
| | | CTTCTTTGATGATGCAATCACTGCTCCGCTTACCG |
| | | ATGCTGATCACGAGCTACACCAATCCAACATGCAA |
| | | GTGGACACTTCAGCAGTGTATACTTGAATTGTTAT |
| | | GCCAACTACAAGAAAGAAAAAATAAAGTTACGTAA |
| | | GTTACCCGTGATATTATATATAGTTTCATATTTTA |
| | | TAAAACAGCTATAATTATAATTATACTCCTTGTCG |
| | | CTTCTCTCACATCATGGCACGTGAGCATGTATATC |
| | | TTGCAAACACCGTAGACGATAGAGATGCCACACTT |
| | | TTCAGGTCTGGTTATCCTATTTTTTTTTTTAAATA |
| | | GGAAGATCTTAGCCCAAGAGGATTCTTCTATATTC |
| | | GTTCACCGGAGATGCCTTCCATTTCACAGCGTGGT |
| | | TCACGTAACAATTCGTTTAGTTCGGAAACTACGGT |
| | | TCCATCGCTCGCTGAGGCCTCTGCTGTCTCGCCCT |
| | | TTGGTCTCCCCACTGACCCAGAATCGCTGTACGGA |
| | | ACGACCCTGACATCGGCCCACACTGTGATCACTAC |
| | | TGTGCCTTATTATTTGTCAGATAGATTGTTTAGTT |
| | | ATGCAGCTCCTGGTGCGGATGGTGCCTTAGATGCT |
| | | GCTGCTCATCTGTGGAGGACATATTTAAGACCTAA |
| | | CGCTCAAGGAAATGTGCCTCATTTAACCAGATTTG |
| | | ATATCAGATCTGGTGCTTCCAATGCCATTTTGGGT |
| | | TATCTGTCAGGGCTAGAGCCTTCCGCTGTGGTGCC |
| | | TGTTTTAGTTCCTGGCGCTGCTTTGACTTATATGC |
| | | GCCCTGTTCTGGCTGAGCGTAGGGACTCACCTGTA |
| | | CCAGTCGCTTTCAATGTTTCTGCATTGGATTATGA |
| | | TTTTGAAACCTCTACCCTGGTGTCCAACTATGTTG |
| | | AACCATTGAATGCTGCCCGTTATTTGGGTTACTCT |
| | | GTGTTCACTCCATTGAGCAAAAACGAGGCTCAAAG |
| | | CATCGCCATTTTAACTCATGCGCTGGCCAACATTG |
| | | AGCCAACCCTCAATTTGTACGATGGCCCTTCTTAC |
| | | CTCAAACAATCTGGAAAAATCGAAGGCATATTAAC |
| | | TGGTGAAAAGCTGTTCCAGCTTTACCAGAAACTGC |
| | | TAGCTGAGATCCCTTCTTGGTCGAAAATAGAGTCC |
| | | TACAAGAGACCTGCTGCTGCTTTAGCCTCCTTGAG |
| | | CAAACTCACCGGTTCTAGACTGAAATCTTTCGAAT |
| | | ACGCCGGCCACAATTCACCTTCGACCGTTTTTGTT |
| | | ATCCATGGATCAGTAGAATCTGAACTTTTGTTGCA |
| | | CACTGTAGAACGCTTTGCTGAGAAAGACGTCCAAA |
| | | TTGGCGCTATTGCAGTTAGAGTTCCGCTCCCCTTC |
| | | AATATTGACGAGTTTGCTTCTTCTTTTCCATCTTC |
| | | TACCAGAAGAATTGTCGTCATTGGCCAGGTTCAAA |
| | | GCTCTTCTTCTTCTTCTTTAAAGAAAGATGTCGCT |
| | | GCCTCTTTGTTCTGGAAACTCGGTGCTTCTGCTCC |
| | | AGCTGTCGCAGAGTTTGTCTATGAGCCAAGCTTCA |
| | | ATTGGAGTAGCGATTCCTTGGAGTCGATTATTGCC |
| | | TCTTATGAAGTCCTTCCAAAATCAACCTCAGCCAC |
| | | CAAAGGAGACTACATTTTCTGGACCGCTGACAATG |
| | | GTCGTTTTGCGGAAGTTGCTTCCAAGATTGCCTAT |
| | | TCCTTTTCACTTAGGGATGACAACAAGCTAAGTTA |
| | | CAGAGCAAAATTTGACAATATCAATGGTGCGGGCG |
| | | TACTGCAGGCTCAACTAAGAACTAATTCTCTTGTT |
| | | GCCACCGATATTGATGCGGCAGACATTGTCTTCGT |
| | | AGAGGGTTTCAAGTTGTTGCAAGCCTTCGATGTGG |
| | | TTTCAACCGCCAAAGAAGGTGCTACGTTAATTATT |
| | | GCATCTTCAGACTCAATTGAAGATTTGGACAAGGT |
| | | TGTAGAGTCATTTCCCACTACTTTCAAACGTGATG |
| | | CTGCTACAAAGAATTTGAAGATTCTTCTCATCGAC |
| | | TTGGCATCTGTTGGTGAGCAGGAAGGTCTTGGTGC |
| | | TAGAACGGGACCAATTGCTTGCCAGGCTATTTTTT |
| | | ATAGGGTTGCTCAACCTGAGTTGGCTGACCAGCTG |
| | | ACTCGTTACTTGTGGGAAGGAGCAGCCTCTGAGAC |
| | | TGAATTATTGGCTTCAGTTGTTGCTGAAGTTATTT |
| | | CCAAAGTTGAAGAAGTTGGTATCAAGGAACTTTCC |
| | | GTCGATAAAGAATGGGCCTCTCTTCCAACAGGGGA |
| | | AGAAGAAGAAGTCATTTTACCCCCTAGACCGCTTG |
| | | AAACTTCATTTGAGCCCAATCTTAGGGAATCTGCA |
| | | ATTGTCCCTCCTCCAGCCATCAGTTCCAAGCTCGA |
| | | ACTCTCAAAGAAACTCGTTTTCAAGGAGAGTTATG |
| | | GTTTGACTAACAGCCTAAGACCTGACTTACCCGTT |
| | | AGGAATTTTATCGTCAAAGTCAAGGAAAACAGACG |
| | | TCTGACCCCCGACGATTACTCACGTAATATTTTCC |
| | | ATATTGAGTTCGATGTCTCTGGTACCGGATTGACT |
| | | TATGACATTGGAGAAGCGCTTGGAATTCATGGTCG |
| | | TAACGACCCTGCACTGGTCGAAGAGTTCATCCAAT |
| | | GGTATGGTCTCAATGGTGAAGACCTTATCGATGTT |
| | | CCTTCTAGAGATGATCCTAACACATTAGAAACCCG |
| | | GACCATCTTCCAGAGTTTGGTGGAAAACATTGATT |
| | | TGTTTGGAAAACCACCTAAACGTTTCTACGAGGCA |
| | | TTGGCTCCATTCGCTCTTGACAGCAGTGAAAAAGC |
| | | TAAATTGGAGAAATTGGCTTCTCCTGAAGGAGCTC |
| | | CGCTGCTTAAGGCTTATCAAGAGGACGAATTTTAC |
| | | TCTTTTGCGGACATTTTGGAACTGTTCCCATCTGC |
| | | CAAACCAACTGCCAGCGATTTGGTTCAGATTGTCT |
| | | CTCCGCTGAAGAGACGTGAATACTCCATTGCTTCC |
| | | TCTCAGAAGATGCATCCTAATGAGGTCCATCTGCT |
| | | CATTGTTGTTGTCGATTGGATTGACAAAAGAGGTC |
| | | GTCAAAGATTTGGACAGTGCTCCCATTACCTTTCT |
| | | GAACTTAGTGTTGGGTCTGAACTGGTTGTCAGTGT |
| | | TAAACCTTCGGTCATGAAGCTGCCACCATTGTCTA |
| | | CCCAGCCTATTGTTATGGCTGGTCTGGGTACAGGA |
| | | TTAGCCCCATTCAAGGCTTTCGTCGAAGAGAAAAT |
| | | CTGGCAGAAGCAACAAGGAATGGAGATTGGTGAAG |
| | | TTTATCTGTATTTGGGTGCTCGTCACCGTAAAGAG |
| | | GAATACCTGTATGGAGAATTGTGGGAAGCTTACAT |
| | | GGACGCCGGAATTGTCACACATGTAGGAGCTGCTT |
| | | TCTCCAGAGACCAGCCTCACAAGATTTACATTCAA |
| | | GATCGTATTAGAGAGAACTTGAAAGAGTTGACCTC |
| | | TGCCATCGCTGACAAGAATGGTTCTTTCTACCTAT |
| | | GTGGTCCAACTTGGCCAGTTCCGGACATTACGGCC |
| | | TGTTTGCAAGATATCATCGAAAGTGATGCTGCTAG |
| | | ACGTGGAGTCAAGGTTGACGCTGACCATGAGATTG |
| | | AGGAGATGAAGGAATCCGGTCGTTACATCTTAGAG |
| | | GTTTATTAGAGAATTATGTAATCTCAAGCATTAAT |
| | | TTCAGTAGATCCCCGCGGCCTTTTCCGCGGCAAAC |
| | | TGTATATTCCCCACCCATCGTGCGATAACAGAGCG |
| | | ATAAGCACAACTGCTAGTATTTATAAGTGATAGCT |
| | | TTCCCATGGTCTTTAGTCTTTGACATGAACTTGTG |
| | | ATGCTGTCTGGATGTGTGATTTCGGAGATTCACCA |
| | | ACAGGAATACGCTAATAATGAGTCCGAGATCTACT |
| | | TGGATAACGCAGGAATGCCCATGTTTGCCAAATCA |
| | | GTGCTGGCTGAATCAATGCAAATGATGATGTTGGG |
| | | TCCTTGGGGCAATCCACATTCACAGTCTTTGGCTT |
| | | CTCAGA |
| 14 | MET10 | MPSISQRGSRNNSFSSETTVPSLAEASAVSPFGLP |
| | protein | TDPESLYGTTLTSAHTVITTVPYYLSDRLFSYAAP |
| | | GADGALDAAAHLWRTYLRPNAQGNVPHLTRFDIRS |
| | | GASNAILGYLSGLEPSAVVPVLVPGAALTYMRPVL |
| | | AERRDSPVPVAFNVSALDYDFETSTLVSNYVEPLN |
| | | AARYLGYSVFTPLSKNEAQSIAILTHALANIEPTL |
| | | NLYDGPSYLKQSGKIEGILTGEKLFQLYQKLLAEI |
| | | PSWSKIESYKRPAAALASLSKLTGSRLKSFEYAGH |
| | | NSPSTVFVIHGSVESELLLHTVERFAEKDVQIGAI |
| | | AVRVPLPFNIDEFASSFPSSTRRIVVIGQVQSSSS |
| | | SSLKKDVAASLFWKLGASAPAVAEFVYEPSFNWSS |
| | | DSLESIIASYEVLPKSTSATKGDYIFWTADNGRFA |
| | | EVASKIAYSFSLRDDNKLSYRAKFDNINGAGVLQA |
| | | QLRTNSLVATDIDAADIVFVEGFKLLQAFDVVSTA |
| | | KEGATLIIASSDSIEDLDKVVESFPTTFKRDAATK |
| | | NLKILLIDLASVGEQEGLGARTGPIACQAIFYRVA |
| | | QPELADQLTRYLWEGAASETELLASVVAEVISKVE |
| | | EVGIKELSVDKEWASLPTGEEEEVILPPRPLETSF |
| | | EPNLRESAIVPPPAISSKLELSKKLVFKESYGLTN |
| | | SLRPDLPVRNFIVKVKENRRLTPDDYSRNIFHIEF |
| | | DVSGTGLTYDIGEALGIHGRNDPALVEEFIQWYGL |
| | | NGEDLIDVPSRDDPNTLETRTIFQSLVENIDLFGK |
| | | PPKRFYEALAPFALDSSEKAKLEKLASPEGAPLLK |
| | | AYQEDEFYSFADILELFPSAKPTASDLVQIVSPLK |
| | | RREYSIASSQKMHPNEVHLLIVVVDWIDKRGRQRF |
| | | GQCSHYLSELSVGSELVVSVKPSVMKLPPLSTQPI |
| | | VMAGLGTGLAPFKAFVEEKIWQKQQGMEIGEVYLY |
| | | LGARHRKEEYLYGELWEAYMDAGIVTHVGAAFSRD |
| | | QPHKIYIQDRIRENLKELTSAIADKNGSFYLCGPT |
| | | WPVPDITACLQDIIESDAARRGVKVDADHEIEEMK |
| | | ESGRYILEVY |
| 15 | MET14 | TCGCTATATTGGAGAAGTCAGCAAGGAAAACGATC |
| | | CAACAAGCCACATCTCTCAAACGCTATTGTTGACA |
| | | GAATCTGTAGTGATGGCACATTTGTACAACAATGA |
| | | CCGAGAGTTTGCATATCTACTGAACGATGGTGTCA |
| | | TTACTAATAAAGTTATAGAGGGAGATACCTCCATT |
| | | AACCGTTTAAAACTGCTTTTCAAGAAATACGGACA |
| | | GGCAATCAGCGATGAAAAAGACACCGAAACTTCCA |
| | | AAGAACAATTAAAGATCCAACTTCTAGACGCAATA |
| | | GAGTCGCTTTAAGCTGGACCCTGACTACCGCACCT |
| | | CACTTCCCAAGAGGATGATTATCGGGGACTGGAAC |
| | | CTGTCTCACTATGGATACCTCACTCCGCAAAGTAT |
| | | CACGTATGAGCACGTGACTACATCTATTTTTCAAT |
| | | ATTCGGGGGACTGTCTACAATGTATATTGTACCTA |
| | | TAATTCCCACTGAATAATCGACAATTCCCACGGAG |
| | | CAAAAGAAAGATGGCTACTAATATCACATGGCATG |
| | | AAAATCTCACTCACGATGAGCGCAAGGAATTGACT |
| | | GAAACAAGGCGGTGTCACTGTCTGCTTACCGGACT |
| | | CAGTGCCAGTGGAAAAAGCACTATCGGTTGTGCCT |
| | | TAGAACAGAGCCTGCTACAGAGAGGAAACAATGCA |
| | | TACAGACTGGACGGTGACAACATCCGCTTCGGGTT |
| | | GAACAAGGACCTTGGATTCAGCGAGGATGATCGTA |
| | | ACGAAAACATCAGAAGAATCAGTGAGGTTTCCAAG |
| | | CTGTTTGCAGACTCTTGTTCTGTTGCTATTACTTC |
| | | ATTCATTTCACCTTACAGGGAAGAGAGAAGAAAAG |
| | | CCAGGGAACTGCACAACAAAGATGGATTGCCATTC |
| | | GTGGAAGTATATGTTGACGTTCCTATTGAGATCGC |
| | | TGAACAAAGAGACCCCAAGGGATTGTACAAGAAGG |
| | | CCAGAGAGGGAATCATCAAGGAATTCACCGGTATT |
| | | TCTGCTCCTTACGAAGCACCTGAGAACCCCGAGCT |
| | | CACGTCCACACAGACAAGCAAACTGTTGAGGAGGA |
| | | GTGCTAAAATCATTATTGATTATTTATTGGAGAAG |
| | | AAACTAATCAAATAGAGTTTGTAGAATAAGATGAT |
| | | TTTTAAGTTTGTATTTCTAGTTCGTGCTGATCTTC |
| | | TTCTCCAATTTCTTCCGTTGAGCGACCAGCATTTT |
| | | GACAGCAGTTAACCATCGGATTAAGTCTTCTTCAT |
| | | TTGGGGCGCAAAACTTGATTCTTTTTTCCCTAGTT |
| | | ATCAGCAAAAAACACCATTTCCTGATCTTGCTCAA |
| | | TGGCTCTAATTCGGTTATATCAATTATGTCATTCA |
| | | GATTGAAAACTTGAAATGGTTTTTCCTCCTTGGAC |
| | | TTGAACATTGACAGCTTCTTGTTAGTCAAGACCAG |
| | | CTTGACAGTTTTCCATTGGTTGTAAGCTTTTTGTT |
| | | TTTCCAATGTTCC |
| 16 | MET14 | MATNITWHENLTHDERKELTKQGGVTVWLTGLSAS |
| | protein | GKSTIGCALEQSLLQRGNNAYRLDGDNIRFGLNKD |
| | | LGFSEDDRNENIRRISEVSKLFADSCSVAITSFIS |
| | | PYREERRKARELHNKDGLPFVEVYVDVPIEIAEQR |
| | | DPKGLYKKAREGIIKEFTGISAPYEAPENPELHVH |
| | | TDKQTVEESAKIIIDYLLEKKLIK |
| 17 | MET16 | CAACTTCCTCACCACCTCCACAAACTCACGCGTGT |
| | | ATATATCAGGGTTTCTACCGTCTTCGATATAATTG |
| | | ACTACGTCCACGGGGATGGGAATGTTCAAATCTGT |
| | | GTTGTGGAGCTTTTGCAAGTGCTCTACAACCTTGT |
| | | TAATGTTGTTGGAAAGACCCAATTGACTTTCCGCT |
| | | GTACCGGCGTAATCGTGCACCTGAACACCCAAATG |
| | | GATGAGGGTTTCGATGAGTTGACTTAGTTCATTTT |
| | | CAACTTGATCTAATGTTGTCGCAGGTGCACTCATA |
| | | CTTGTCATGGAGAATGAAAGTAAGTTGATAGAGAG |
| | | CAGACTTCGAGGATGGGATGAACTTGATTAGGTAA |
| | | TCTTTGACAATGTCTTAGAGGTAGGCAGAGGATGC |
| | | TGGAAAAAAAAAATTGAAAACGCCCAAGCTTCCAG |
| | | CTTTGCAAGGAAAGAAGAAAAGGGAGTTGCCAGCA |
| | | CGAAATCGGCTTCCTCCGAAAGGTTCACAATTGCA |
| | | GAATTGTCACCATTCAAATGCCTTTACCCTTCATC |
| | | TGTGGTACCTCAGGCTAAGAACGGGTCACGTGATA |
| | | TTTCGACACTCATCGCCACAATATGTACTAGCAAG |
| | | AACTTTTCAGATTTAGTAATCCGTTCGAAACGGGA |
| | | AAAAATGTTTTTACCCTTCTATCAACTGCTAATCT |
| | | TTCTAGGTTTATACTGCCAGCAGCCCGTTCCAGAT |
| | | ACCAACATGCCATTCACTATAGGCCAGTCAAAAAC |
| | | CAGTTTGAACCTCTCCAAGGTCCAAGTGGACCACC |
| | | TTAACCTTTCTCTTCAGAATCTCAGTCCAGAAGAA |
| | | ATCATACAATGGTCTATCATTACCTTCCCACACCT |
| | | GTATCAAACTACGGCATTCGGATTGACTGGGTTGT |
| | | GTATAACTGACATGGTTCACAAAATAACAGCCAAA |
| | | AGAGGCAAAAAGCATGCTATTGACTTGATTTTCAT |
| | | AGACACCTTACATCATTTTCCACAGACTTTAGATC |
| | | TCGTTGAACGAGTCAAAGATAAATACCACTGCAAT |
| | | GTTCATGTCTTCAAACCACAGAATGCCACTACTGA |
| | | GCTCGAGTTTGGGGCGCAATATGGCGAAAACTTAT |
| | | GGGAAACAGATGATAACAAGTATGACTACCTCGTA |
| | | AAAGTTGAACCCTCACAACGTGCCTACCATGCATT |
| | | AGACGTCTGCGCCGTCTTCACAGGAAGAAGACGGT |
| | | CTCAAGGTGGTAAAAGGGGAGAATTGCCCGTGATT |
| | | GAAATTGATGAAATTTCTCAGGTGGTCAAGATTAA |
| | | TCCGTTAGCATCCTGGGGGTTTGAACAAGTTCAAA |
| | | ACTATATCCAAGCTAATAGCGTTCCATACAACGAA |
| | | TTGCTGGATTTGGGATACAAGTCAGTTGGAGATTA |
| | | CCATTCCACACAACCCACTAAAAATGGTGAAGATG |
| | | AAAGAGCAGGCAGGTGGAGAGGTAAACAAAAGAGT |
| | | GAGTGTGGTATCCACGAAGCTTCTAGATTTGCACA |
| | | ATATTTGAAAGCTCAGCAAAACATATGAATATAAT |
| | | TTTTTTTTTCTCTACACTATTTATCCTGTAAGTTT |
| | | CTGTTTCCCCATGTAGGATCTTTTTCTCCTTCTCT |
| | | GTCTCCCATTTTTTTTGTTCCCTGTAGTCTTGCCT |
| | | TGCCTGAGATGCGAGCTCGTCCGCCCATCCAGTCG |
| | | TGTGAAGGGCCTAGCTTTTCAAAAAGAAAATACCT |
| | | CCCGCTAAAGGAGGCGTTGCCCCTTCTATCAGTAG |
| | | TGTCGTAACCAATTTTCACAAACAATAAAAAAAGG |
| | | ACACCAACAACGAAATCAACTATTTACACACATCC |
| | | AGATCCGTCCCCCTCCCCATCCAAGAGTTAAAGAC |
| | | AAATATGGCTGTTAATAATCCGTCT |
| 18 | MET16 | MFLPFYQLLIFLGLYCQQPVPDTNMPFTIGQSKTS |
| | protein | LNLSKVQVDHLNLSLQNLSPEEIIQWSIITFPHLY |
| | | QTTAFGLTGLCITDMVHKITAKRGKKHAIDLIFID |
| | | TLHHFPQTLDLVERVKDKYHCNVHVFKPQNATTEL |
| | | EFGAQYGENLWETDDNKYDYLVKVEPSQRAYHALD |
| | | VCAVFTGRRRSQGGKRGELPVIEIDEISQVVKINP |
| | | LASWGFEQVQNYIQANSVPYNELLDLGYKSVGDYH |
| | | STQPTKNGEDERAGRWRGKQKSECGIHEASRFAQY |
| | | LKAQQNI |
| 19 | MET17 | CCCAGTATGAGAGGAACAGGAGATGAGCTGGAATT |
| | | TGGAAACAGGAACGTTCAATTGCCAAGGAGAAGTT |
| | | TGAGAGGAGAGAGTGGCAAAGAGAATGGAGTCACT |
| | | TCCTATCCATGCTTACAACAAGATCTCTGGAATAT |
| | | GACATACAACATAGCAACAAAGAGGGGGTGCATCA |
| | | AAAAAAAATTACACGTTTTCCCACCCTTTCCAACG |
| | | AACCCCCACACCAGTGAGGTGAACAGATTTAACGG |
| | | GTCTCAGATAAACGAAAAAATGCTAACAATACCAT |
| | | CTATCGTGAGGGGGCGGCCCACTGCCACATTTCCA |
| | | AAAGATACCCCCCTCCGCTTCAGATTGTAATTGTC |
| | | TGTTTTATAGTACTGCAGTGAAGCGCCACAGCTCC |
| | | AAAACTTAATTTGACTTCTTTATCAATTACCGTAA |
| | | TATTAGTCGGGCCTTGCCGCATCACGTGACCCGAT |
| | | TTCACTATAAAACTCTCCGTTCCCATAAAGTTTTA |
| | | CCACATCACGTGAGTTGTCAACATTGAAACCCCTC |
| | | GATGTAATGCTTCACAGGTTGGTTATTTAAATCAT |
| | | CCAATCGCCGACCAAATGAAATGATTTCTAACGTT |
| | | TCCTTATTCACATACAAAGATGCCTTCTCACTTCG |
| | | ACACTTTGCAGCTGCACGCCGGTCAGACCGCTGAA |
| | | GCTCCACACAATGCCAGAGCTGTTCCTATCTACGC |
| | | TACCTCGTCTTACGTTTTCAGAGACTCTGAGCACG |
| | | GTGCCAAGCTGTTCGGTTTGGAGGAGCCAGGTTAC |
| | | ATCTACTCTCGTTTGATGAACCCTACTCAGAACGT |
| | | CTTTGAAGAGAGAATTGCCGCTTTGGAGGGTGGTG |
| | | CCGCTGCTTTGGCTGTTGGATCCGGTCAAGCTGCT |
| | | CAATTCCTGGCTATTGCTGGTTTGGCTCACACTGG |
| | | TGACAACGTCATCTCCACCTCTTTCTTGTACGGTG |
| | | GAACTTACAACCAATTCAAGGTCGCCTTCAAGAGA |
| | | CTGGGAATTGAATCCAGATTTGTCCATGGTGATGA |
| | | CCCAGCTGAATTCGAGAGACTGATCGATGATAAGA |
| | | CCAAGGCCATCTACGTTGAGTCCATTGGTAACCCA |
| | | AAGTACAATATTCCAGATTTTGAGGCTCTCGCAGA |
| | | GCTTGCCCACAAGCACGGTATCCCATTAGTTGTTG |
| | | ACAACACCTTTGGTGCCGGTGGTTACTACGTTAGA |
| | | CCAATCGAGCTTGGTGCTGACATCGTCACCCACTC |
| | | CACCACTAAGTGGATCAATGGTCACGGTAACACCA |
| | | TCGGTGGTGTTGTCGTTGACTCTGGTAAGTTCCCA |
| | | TGGAAAGACTACCCAGAGAAGTACCCTCAATTCTC |
| | | CAAGCCATCTGAGGGTTACCACGGTTTGATCTTGA |
| | | ATGACGCCTTTGGACCAGCTGCCTTCATTGGTCAC |
| | | TTGAGAACTGAACTGCTAAGAGATTTGGGTCCTGC |
| | | TTCAAGTCCATTCGGTAACTTCTTGAACATAATCG |
| | | GTTTGGAGACCTTATCTCTGAGAGCTGAGAGACAC |
| | | GCTGAGAATGCTTTGAAGCTGGCCAAATACTTGGA |
| | | AACCTCTCCATACGTCAGCTGGGTCTCTTACCCTG |
| | | GTTTGGAGTCTCACGACTACCACGAGGCCGCTAAG |
| | | AAGTACTTGAAGAACGGTTTCGGTGCTGTATTGTC |
| | | TTTTGGAGTCAAGGATCATGGCAAGCCAGCGCTCA |
| | | CTCCCTTCGAAGAGGCTGGTCCTAAGGTTGTAGAC |
| | | TCCCTGAAGGTTTTCTCCAACTTGGCTAACGTTGG |
| | | TGACTCCAAGTCTTTGATCATTGCTCCTTACTACA |
| | | CTACTCACCAACAGTTGTCTCACGAGGAGAAGCTG |
| | | GCTTCCGGTGTCACCAAGGACTCTATCCGTGTTTC |
| | | TGTCGGAACAGAGTTCATCGATGATCTTATTGCAG |
| | | ACCTTGAACAGGCCTTTGCCCTTGTTTACGAGGAG |
| | | GCAAACACAAAGTTGTGAGTTAGTTTAACAGTTGT |
| | | AATTGATCAATAATGTATGTGTAGAGTTTAGAATA |
| | | CGATAATGTGTATATCATTATGTCATTTCCATTGA |
| | | TAGTAACTATTGGTAAGTAGCACAGCTATTTGTAT |
| | | GTATATAATTTGAGTAATCAAGGTTAAATGTAAAA |
| | | ATAAATATAAGTGTCATCATCGTTGTCTTTGACAG |
| | | TAAGAACTAGTTAATCATCTCCGTGTTTGAAGCAG |
| | | CATCTTTTACCGTAGCGGCATTTGCCGAACTTGGT |
| | | CCAGTTGGCACAAGGTTTCGTCTTCCAGTTGGAAG |
| | | GTCTCTTCACGGACTTCAGTTCGTGAGTCCCGTGA |
| | | GCAAATTGACACTTT |
| 20 | MET17 | MPSHFDTLQLHAGQTAEAPHNARAVPIYATSSYVF |
| | protein | RDSEHGAKLFGLEEPGYIYSRLMNPTQNVFEERIA |
| | | ALEGGAAALAVGSGQAAQFLAIAGLAHTGDNVIST |
| | | SFLYGGTYNQFKVAFKRLGIESRFVHGDDPAEFER |
| | | LIDDKTKAIYVESIGNPKYNIPDFEALAELAHKHG |
| | | IPLVVDNTFGAGGYYVRPIELGADIVTHSTTKWIN |
| | | GHGNTIGGVVVDSGKFPWKDYPEKYPQFSKPSEGY |
| | | HGLILNDAFGPAAFIGHLRTELLRDLGPASSPFGN |
| | | FLNIIGLETLSLRAERHAENALKLAKYLETSPYVS |
| | | WVSYPGLESHDYHEAAKKYLKNGFGAVLSFGVKDH |
| | | GKPALTPFEEAGPKVVDSLKVFSNLANVGDSKSLI |
| | | IAPYYTTHQQLSHEEKLASGVTKDSIRVSVGTEFI |
| | | DDLIADLEQAFALVYEEANTKL |
| 21 | MET19 | GGTGAAAAATACCAAGGGCGATGGAAATTTCAAAG |
| | | GCCGATCTGGGGATGTGTGGGGTAAAGACTTTGGA |
| | | TGGAATCCAGGGGCAAAGACAAGGGCTAGACTTCA |
| | | CTATATTGGTGGTAAAAGTGAATCTACTAGAAGTT |
| | | TGAGTCAACGACGATATGGAGTAACCAAGTGAAGA |
| | | CGATATCTTTAGTTCGTTATGGCCACCTTAAAAGA |
| | | AGCCCACTCAGTCCATGTGAGTTCTGAAACTTTTA |
| | | AAGACAGTTAACCCAAGGTTCACAATTGTGTGACC |
| | | TTATGTCAACTGTACTAGAAGGCCAAAGATTATTG |
| | | GACGATTGGGTTATCTATTTCCTTGATAAGCATGT |
| | | GCTCCAATCAATACACCCACCTGTCAGGGGATACA |
| | | CAGTGCGGAGCTCCGTTTTCTCCCAGAAATTCGGT |
| | | TGGAGCTCTTTTCTTAAACTTCGAAAGTCCCCCGA |
| | | CAGAGAAGTGCCGTTAGCCAATAGTGTCCCTGCAT |
| | | TCTGGTTCCTCCCCACTGCAGCGTCAGCTGGAAAG |
| | | GGCTCTATTCTAAGCTATTCTAAAGCAATCCAAAG |
| | | GTGGGGGTCGGATCAATGCGCGATCTTTCGTCGCC |
| | | AGTGTCGGGGCCCGGCACGGGGGCCGTAACCGGCT |
| | | TTTCTCTAGGTTGACACCATGGGATATCCCCTGAT |
| | | TGGGCAAATCCCACATAAGTATGGCTTGCGGCTTA |
| | | CTAATCGCGTAAGTCGCGCATTCTCTTTTTCCTGA |
| | | TCCTTAATATCAATCCTCCGGCACCATCATCGTAG |
| | | TTTGCGAGATTCCATAAACTTTTTGGCCCCCTAAC |
| | | TTTTTTTTTGTTGCCATCCTTTACTTCCATCTAAA |
| | | AAAACCGACACAGAATCTGCCAAACAATGACCGAT |
| | | ACGAAAGCCGTAGAATTTGTGGGCCACACAGCCAT |
| | | TGTAGTCTTTGGAGCTTCAGGGGACCTGGCTAAGA |
| | | AGAAGACTTTCCCTGCCCTCTTCGGACTTTACCGT |
| | | GAGGGATACCTGTCCAACAAGGTGAAGATTATTGG |
| | | CTATGCTAGATCAAAGCTGGATGACAAGGAGTTCA |
| | | AGGATAGAATTGTGGGCTATTTCAAGACAAAGAAC |
| | | AAGGGCGACGAGGACAAAGTTCAAGAATTCTTAAA |
| | | GTTGTGCTCATATATTTCAGCTCCTTATGACAAAC |
| | | CAGATGGGTATGAAAAGTTGAATGAAACTATTAAC |
| | | GAATTCGAAAAGGAAAACAACGTCGAACAGTCTCA |
| | | CAGGTTGTTCTACTTAGCTTTGCCCCCTTCTGTTT |
| | | TCATACCTGTTGCTACGGAGGTCAAGAAGTATGTT |
| | | CATCCAGGTTCTAAAGGGATTGCTCGGATTATCGT |
| | | GGAAAAACCTTTCGGGCACGACTTGCAGTCAGCAG |
| | | AAGAGCTTTTGAATGCTTTGAAGCCGATCTGGAAA |
| | | GAAGAGGAATTGTTTAGAATCGACCACTATCTAGG |
| | | TAAGGAGATGGTTAAGAATTTGTTGGCCTTCCGTT |
| | | TTGGAAACGCATTCATCAATGCTTCTTGGGACAAC |
| | | AGACATATCAGCTGTATCCAAATCTCGTTCAAGGA |
| | | GCCTTTTGGAACAGAAGGTCGTGGTGGCTATTTTG |
| | | ACTCAATTGGTATAATAAGAGACGTCATTCAGAAC |
| | | CACTTGCTTCAAGTGTTAACCCTCTTAACCATGGA |
| | | GAGACCCGTCTCTAATGACCCTGAGGCTGTTAGAG |
| | | ATGAAAAGGTTCGCATTCTGAAGTCAATTTCTGAG |
| | | CTAGATTTGAACGACGTTTTGGTGGGTCAATACGG |
| | | CAAATCTGAGGATGGAAAGAAGCCAGCTTATGTGG |
| | | ATGATGAAACTGTTAAGCCAGGTTCTAAATGTGTC |
| | | ACATTTGCAGCCATTGGCTTGCACATCAACACAGA |
| | | AAGGTGGGAAGGTGTCCCAATCATTTTAAGAGCTG |
| | | GTAAGGCTTTGAACGAAGGTAAAGTTGAGATTAGA |
| | | GTGCAATACAAACAGTCTACTGGATTTCTCAATGA |
| | | TATTCAGCGAAATGAATTGGTCATCCGTGTGCAGC |
| | | CTAACGAAGCCATGTACATGAAACTGAACTCCAAA |
| | | GTCCCAGGTGTTTCCCAAAAGACTACTGTCACTGA |
| | | GCTAGACCTCACTTACAAAGACCGTTACGAAAACT |
| | | TTTACATTCCAGAGGCATATGAATCACTTATCAGA |
| | | GATGCTATGAAGGGAGATCACTCTAATTTTGTCAG |
| | | AGATGACGAGTTGATACAAAGTTGGAAGATTTTCA |
| | | CTCCTTTACTGTATCACTTGGAGGGCCCTGATGCA |
| | | CCGGCTCCAGAAATCTATCCCTACGGATCCAGAGG |
| | | TCCAGCTTCATTGACCAAATTCTTGCAAGATCATG |
| | | ATTACTTCTTTGAATCACGCGACAATTACCAATGG |
| | | CCAGTGACAAGACCCGATGTGCTGCACAAGATGTA |
| | | AATTATTCTATAGATTTAGGACGATTACAGATATC |
| | | AATGATAGTTTAGCTTGTTTCAGTATTACGTAATA |
| | | AATGACTCAGAGGTATCTCAGGATCTGTGGGGCAG |
| | | GAAGTGGCATTGCATTTGCTCGCTCCTATTAGCTT |
| | | ATCAGGGAAGAGGAAAGAAAAATTCTTGCATATAA |
| | | AGTGCTGGGCCAGCCCACATCCTTAGCACGTTATC |
| | | AGCTTTTCACAACTCTACTCCTGATTTTCTGATGG |
| | | AAACCCCAAGCTATCCACTGAAAGCAAAAACCAAA |
| | | GATGAAGGGGAAATAATTGTAAGGGATATCATTCT |
| | | AACTAACCACGAAGAGACACAGGGTCATTCTTC |
| 22 | MET19 | MTDTKAVEFVGHTAIVVFGASGDLAKKKTFPALFG |
| | protein | LYREGYLSNKVKIIGYARSKLDDKEFKDRIVGYFK |
| | | TKNKGDEDKVQEFLKLCSYISAPYDKPDGYEKLNE |
| | | TINEFEKENNVEQSHRLFYLALPPSVFIPVATEVK |
| | | KYVHPGSKGIARIIVEKPFGHDLQSAEELLNALKP |
| | | IWKEEELFRIDHYLGKEMVKNLLAFRFGNAFINAS |
| | | WDNRHISCIQISFKEPFGTEGRGGYFDSIGIIRDV |
| | | IQNHLLQVLTLLTMERPVSNDPEAVRDEKVRILKS |
| | | ISELDLNDVLVGQYGKSEDGKKPAYVDDETVKPGS |
| | | KCVTFAAIGLHINTERWEGVPIILRAGKALNEGKV |
| | | EIRVQYKQSTGFLNDIQRNELVIRVQPNEAMYMKL |
| | | NSKVPGVSQKTTVTELDLTYKDRYENFYIPEAYES |
| | | LIRDAMKGDHSNFVRDDELIQSWKIFTPLLYHLEG |
| | | PDAPAPEIYPYGSRGPASLTKFLQDHDYFFESRDN |
| | | YQWPVTRPDVLHKM |
| 23 | MET22 | TGCCATGGGCTTTTGTCACTGGGTTGTAAGCCTCT |
| | | AGCCATTCGGGGTCATCTTCACTACCTATGACGTG |
| | | AAAAAAGTCTCCTTTCTTGAAAGTGAGCTCACCAG |
| | | GGCCCTGGGCCTTGTAGTCATACAGAGATTTGATG |
| | | ACTTTTTTGGGCGTATCGAGAACCTCGGAGTGGGA |
| | | GGTATCGACTTGTATTGGTTCAGCCTTGGTGATCT |
| | | TGGGACCCTTAGAATGCTTGTCTTTAGAAGATCTT |
| | | TTGAAACTTATCATTGGAAGAGATTGGTATGAAAT |
| | | GAGAGACTTTATGAATAGCTTGACAAGAGAAGAGG |
| | | GAAGGGAGAGAAAAGGAGTCGATCACTGTGAAAGT |
| | | AATTTCCTTTCAGGTAATTACGAATGTTGAGAGTG |
| | | AGAATGACAAGAATGGTGCTGGGATGCAATATTCC |
| | | GTACCTTTCTGCATCACCCCCTCTCAAGTACGAGT |
| | | TGTCCACCTGCAAGAAAAAAAAGCACTGCGTTCAG |
| | | GAGAAAAAATATGTTCAGCAGGGAAGTTAAGCTAG |
| | | CCCAATTGGCTGTCAAAAGGGCATCTCTATTGACT |
| | | AAGAGGATAAGTGATGAGATTGCAGCTCGCACAGT |
| | | TGGCGGAATTTCGAAATCGGACGATTCTCCAGTCA |
| | | CTGTGGGGGACTTTGCTGCTCAGTCTATCATCATC |
| | | AACAGCATCAAGAAAGCCTTCCCCAATGATGAGGT |
| | | TGTTGGAGAAGAAGACTCTGCGATGTTGAAGAAAG |
| | | ACCCAAAGCTGGCTGAAAAGGTGTTGGAAGAGATC |
| | | AAGTGGGTTCAAGAGCAGGACAAAGCCAACAATGG |
| | | GTCGTTATCTCTGTTGAACTCGGTAGACGAAGTTT |
| | | GCGATGCTATCGACGGCGGCAGCTCTGAAGGTGGC |
| | | CGTCAAGGAAGAATTTGGGCCTTGGATCCCATTGA |
| | | TGGTACTAAGGGCTTCCTGAGAGGCGACCAATTTG |
| | | CCGTTTGTCTGGCATTAATCGTGGATGGGGTTGTA |
| | | AAAGTTGGTGTAATTGGGTGTCCAAATCTACCGTT |
| | | TGACCTACAAAATAAGAGCAAGGGAAAAGGAGGAC |
| | | TTTTCACCGCAGCTGAAGGCGTAGGATCATACTAT |
| | | CAGAACTTGTTTGAAGAGATCTTGCCTCTGGAATC |
| | | ATCAAAAAGAATCACAATGAACAATTCTCTTTCTT |
| | | TTGATACCTGCAGAGTCTGTGAAGGTGTTGAGAAG |
| | | GGTCATTCAAGTCATGGGTTGCAAGGATTAATAAA |
| | | AGAAAAGCTCCAGATCAAGTCCAAGTCCGCCAACT |
| | | TGGATTCTCAAGCCAAGTACTGTGCTCTGTCGAGA |
| | | GGAGATGCTGAAATATATTTGAGGTTGCCAAAAGA |
| | | TGTGAATTACCGAGAGAAAATATGGGATCATGCTG |
| | | CTGGCAACATTCTGATCAAGGAAAGCGGAGGCATT |
| | | GTGTCTGATATTTATGGTAACCAGTTGGATTTTGG |
| | | CAACGGTCGGGAGCTCAACTCGCAGGGAATAATCG |
| | | CGGCATCAAAAAATTTACATAGCGATATCATCACT |
| | | GCAGTGAAAAGTATTATTGGAGATAGAGGCCAAGA |
| | | TTTGGAGAAGTATATATAGATATAGCTTGTACTAG |
| | | AATATGATCACGAGGCTAAAGAACAAAAGTAAGGA |
| | | GAGGACAGCCGCTTTGAAGGGCAAAAAGCGGGCAC |
| | | AGGAAGGTATTGAAGCGCAAGAACGGAAAGATCTA |
| | | CCACCCAGTAAGATTACGCAAAGGACGAAGAGCTC |
| | | AAATAAAGTCACCAAGATGGGAAAACAGAGCTGGT |
| | | ATAACGATCTTTCAAAGTACAATCACATTAAACCA |
| | | TTGACGTCCAAAGTTAGAGGAATGGTCAGTAATAT |
| | | GACTAATTACAATCATCTCTTGATGAGATCTATTG |
| | | AGAATCCTCACTATAGACAGAAACTATTAGACATT |
| | | GAAGAAAGGAAGCTGCGCTTGAATAGCTATCCGCT |
| | | GCCCAAGGTACAAAATGACCAGAGCTTGAAAGATG |
| | | CCTTGAACCACTTTAGAATTGATAGACAGGGCAGA |
| | | TCAATTCCGATACTGGATAGAAATCCTCATGTGTG |
| | | TTCTTCATTCAAAGAGAATAAGCATT |
| 24 | MET22 | MFSREVKLAQLAVKRASLLTKRISDEIAARTVGGI |
| | protein | SKSDDSPVTVGDFAAQSIIINSIKKAFPNDEVVGE |
| | | EDSAMLKKDPKLAEKVLEEIKWVQEQDKANNGSLS |
| | | LLNSVDEVCDAIDGGSSEGGRQGRIWALDPIDGTK |
| | | GFLRGDQFAVCLALIVDGVVKVGVIGCPNLPFDLQ |
| | | NKSKGKGGLFTAAEGVGSYYQNLFEEILPLESSKR |
| | | ITMNNSLSFDTCRVCEGVEKGHSSHGLQGLIKEKL |
| | | QIKSKSANLDSQAKYCALSRGDAEIYLRLPKDVNY |
| | | REKIWDHAAGNILIKESGGIVSDIYGNQLDFGNGR |
| | | ELNSQGIIAASKNLHSDIITAVKSIIGDRGQDLEK |
| | | YI |
| 25 | MET27 | ATTCTCTTTGGGGTTTGTCTAGCGGCTAATCTGAA |
| | | CATTTTGTGTTTGTTGCAAGGTAATAGAACTAAAG |
| | | AGAGTTACTATTGGAGAGGTATCGTGCAAGAAAAG |
| | | AGTAGTCCGGGTAACAACGATCAATAGTAGGAGGT |
| | | GAGAGGTCACCTCATAGAATTTCGTGTATTTCCTT |
| | | TACGCTTTTTGCCAATCTTCTGATTGGCTGGATCC |
| | | CCCAAAATATGTCGCGCGCAGCCTCTCACTGGAGG |
| | | GCCAGTCGGCCCATATTCACGTGACGCACCTTCGA |
| | | ACCCAAAGGGTAAGCTAACTAACCAAGAAAATACT |
| | | ACTTTCCCTTTTCAAATACCAACACATAGAAACAA |
| | | TGGCTGCAGCTTCATTAACCAGAATTCAAGGATCT |
| | | GTCAAGAGAAGAATCTTGACCGACATCTCAGTTGG |
| | | CCTGACCCTCGGTTTCGGCTTTGCTTCCTACTGGT |
| | | GGTGGGGAGTCCACAAGCCAACCGTAGCCCACAGA |
| | | GAGAACTACTACATTGAGTTGGCTAAGAAGAAGAA |
| | | GGCCGAGGAAGCTTAACTTATTTAAACCTGTGACA |
| | | AAGATCAAGAGCTGCACAGTACTTTATATTGTGTA |
| | | TTTTTAAAGAGCATATTTTGCATGACTTTTATTGG |
| | | TGAACACGGAGATGGACTGTGTCTTTGATGATGCT |
| | | AGCGTGGTATTGCAAGGTGAAATTAATGGTTTTGG |
| | | AGGGCAGATTTTAGTTTAGCAAACTTCTTGCCTTG |
| | | CGAGTGACCGTCCGCTGTCCAATCCAAATACTTGT |
| | | AGAATTTTCTGACCTGGTTCTCCCCAGTCAACCTA |
| | | GAAATTTGCTGACATGAGCCCTTCAAATGAAGAAC |
| | | GTTGATACTTTAAAACTGGTGGCTATGCTGTTATT |
| | | AACCCTGGTATATTCTCTGATTTCTGAGCTAAAAC |
| | | ATGGAAGGTGGAAAGTAGCCTTTTTGCTCCCAAGA |
| | | GCACCCAAAGTGACTCTCGAAATAATTCTTATCCA |
| | | AAAGTAATTTGTTAACACTGATGATAGATCTCAGC |
| | | TCAGTTGATTCCAAGCCAGTCGATGATCTGTTTGC |
| | | AATCTTTGACGAGATCAATCGAAAGCTTAACATAC |
| | | AATGCGATCATCTGCTGATCTTGGAAAAAAAACTA |
| | | TCTCAGCCAATCAACTTTTTGACGCCGTTCAGCGC |
| | | TCTTCAAAAGGTCACCAGAATAACCAAGGTCATAT |
| | | GGTTAGAGAACCTTACCGATGAAACTTTGCATGCA |
| | | GCTCTGAATGAATTTAATTCTGTTGTGTTCTTCTG |
| | | CGAGGATAGTTTGCAAAACGTTGGACGGGTGGCAA |
| | | AACTGTTCCGATCCACCATTCTACCCATCACTGAG |
| | | ACGAATTCAATGATGAACACATCACTAATAACTCT |
| | | GGGATCCTTAAACCAATCAATTCGTCTATATCTGT |
| | | CAGAGCTATCATTGGAGAATGACATTGACTACTAT |
| | | TCGTGGGATTCTATTCTGTTCAGAATAGACAAAGA |
| | | TCTACTTTCTCTAAATTCTTCCTCAGATTTGAAAA |
| | | AGTTGTACCAATTGCAATCTATCGAACCTTTGTAT |
| | | GCCCTGGCAAATGGTTTGCTGCATTTGGTGATTCA |
| | | TTCTAACTTCAAGTTAAGATTCACAAATAAATTTA |
| | | TCAAGGGTGCCAATTCAGCCAAGTTTTATGATATC |
| | | TATCAGAAATTATACACCAACTACACTCTGAATAA |
| | | ATTGAGTCCGGAAAAAAGAAAAATCCTGGAAGATG |
| | | TGGACGAGACATTGTTCATGGATATTCACTCATTC |
| | | TACAACAATCAATGCGACCTGTTTGTTTTTGAGAG |
| | | AAGCGTTGATTTTATAACCCCGTTATTAACACAAC |
| | | TCACATACTGTGGTTTGGTGCATGATAACTTTAAC |
| | | GTTGAATACAACACCGTCAACTTGAAATCTGAAAC |
| | | GATACCACTGAATGATGAGCTCTACCAGGAAATCA |
| | | AAGATTTAAATTTCACTGTTGTGGGATCTTTGCTC |
| | | AATTCTAAAGCTAAATCGTTACAAGAATCATTTGA |
| | | AGAAAGGCACAAGGCTAAAGACATTGCACAAATAA |
| | | AGGATTTTGTTTCCAACTTAACGAACCTCACAAAG |
| | | GAACAACAATCGTTGAAGAATCATACTAACTTGGC |
| | | TGAGGCAGTTCTAGCAAAAGTACATGATGAAACGG |
| | | GCAACAGTGAAAACCACTCGGAGGACAGCTTGTTC |
| | | AATCAGTTCTTGGAACTCCAACAAGATATCTTATC |
| | | CAACAAACTAGACAATAAAACCACCTACAAATCAA |
| | | TTCAAACTTTTTTCTGCAAATACAACCCTCCTCCT |
| | | TTGCTACCTCTTAGGTTGATGATCCTCTCCTCAAT |
| | | TGTTAAGAATGGGATAAGGGATTATGAATTTAATG |
| | | CATTGAAGAAGGATTTCGTTGATTACTATGGTGTG |
| | | GACTATCTTCCCGTAATAAACACGCTTGCCGAGCT |
| | | CTCACTTTTGACAAGTAAGAAGAGCCAGCCCTTAG |
| | | AACAAAATCCTAATTCACAACTCATCAAAGACTTC |
| | | CATAATTTGAGCACTTTTCTGAACCTTTTGCCTGG |
| | | AACGGAAGAAACAAATCTTCTAAACCCTACCGAAT |
| | | TAGATTTTGCTCTCCCAGGGTTTGTTCCTGTCATT |
| | | ACTAGATTAATTCAGTCGGTTTATACCCGATCTTT |
| | | CATTGGGCCGAATTCCAATCCTGTAATTCCATACA |
| | | TTGCGGGATCTAACAAAAAGTACAACTGGAAGGGT |
| | | CTCGATATCATCAACACATACTTGACTGGTACCAT |
| | | GCAGTCCAAACTGTTGATACCAAAATCAAAAGAGC |
| | | AAATATTCACCCACAGAACTGCAGCGCCTCCTCAT |
| | | TCACGTAAGGGTGTTCTCAGAAATGAGGAGTATAT |
| | | TATAGTAGTCATGCTGGGAGGTATATCGTACGGAG |
| | | AATTGTCAACCTTAAGGGTCGCCATATCGAAGATC |
| | | AACGAGTCTATGAACTTGAACAAAAAGCTTCTTGT |
| | | GCTCACAAGTTCTGTTCTCAAAAGTGATGATATAA |
| | | TCAAGCTGACTAAATAATATTGTTGCCCTATTAAC |
| | | GACTGTACAGTTCATATCTCCTTCGCTTCGATTCC |
| | | TATCCCTGACTTTCCCTTACAGAGATAGAGTTAGA |
| | | TGCCTTTAGAATCAGATACTCTAGTATTATCGCGC |
| | | GCAGTAAGTGCTCCTAAATTTTCTTTTTTTTCTGG |
| | | TTTCAAACTTAGTTAAGAAAGAGTGGACATGAGAA |
| | | ACCTTGTGGTCCTGAACAAAGGAGAGATCGTGGTT |
| | | GAATCACGAACCTATCCTGAGTTGAGAGTGCTGGA |
| | | TTCAGTATTTGACTCCATTTCAGACACAATTACCG |
| | | TGGCACTTGGTAAGAATGAATCTGGAATAATTGAA |
| | | GTTCACCAGTTCATG |
| 26 | MET27 | MIDLSSVDSKPVDDLFAIFDEINRKLNIQCDHLLI |
| | protein | LEKKLSQPINFLTPFSALQKVTRITKVIWLENLTD |
| | | ETLHAALNEFNSVVFFCEDSLQNVGRVAKLFRSTI |
| | | LPITETNSMMNTSLITLGSLNQSIRLYLSELSLEN |
| | | DIDYYSWDSILFRIDKDLLSLNSSSDLKKLYQLQS |
| | | IEPLYALANGLLHLVIHSNFKLRFTNKFIKGANSA |
| | | KFYDIYQKLYTNYTLNKLSPEKRKILEDVDETLFM |
| | | DIHSFYNNQCDLFVFERSVDFITPLLTQLTYCGLV |
| | | HDNFNVEYNTVNLKSETIPLNDELYQEIKDLNFTV |
| | | VGSLLNSKAKSLQESFEERHKAKDIAQIKDFVSNL |
| | | TNLTKEQQSLKNHTNLAEAVLAKVHDETGNSENHS |
| | | EDSLFNQFLELQQDILSNKLDNKTTYKSIQTFFCK |
| | | YNPPPLLPLRLMILSSIVKNGIRDYEFNALKKDFV |
| | | DYYGVDYLPVINTLAELSLLTSKKSQPLEQNPNSQ |
| | | LIKDFHNLSTFLNLLPGTEETNLLNPTELDFALPG |
| | | FVPVITRLIQSVYTRSFIGPNSNPVIPYIAGSNKK |
| | | YNWKGLDIINTYLTGTMQSKLLIPKSKEQIFTHRT |
| | | AAPPHSRKGVLRNEEYIIVVMLGGISYGELSTLRV |
| | | AISKINESMNLNKKLLVLTSSVLKSDDIIKLTK |
| 27 | MET28 | ACAAACATAAGAAAAAATCCAAGAATAAGAGCAAG |
| | | AATGTCAGGTTTTTGGACGACCTGGAATCCAACCT |
| | | GGATCTTGACAACACAGACGATAAGAAGGACAATA |
| | | GTGTGATGAGCAAACTTCTCAGCTCAATGGGCTAC |
| | | CAGGCGCAAGAACCTTACAAACCGCTAGATAAGGG |
| | | TGCAAACGCCGATCTTGACATTGAGATGGACAGTC |
| | | ATGGTACCTCGGAAAAGTAGGGCTAAGCCAACCAA |
| | | TGAAATGTATAGAGTATGTTGAAAAGGTGTTAGGT |
| | | GAATAATATTAAAAGTGTACTATTCGACTCCGGCG |
| | | TTTTTCCACGCTTTGAAATTTTCCATAGCCTACCG |
| | | CTTACAAAAGTTGACTCTGTCACCCCCCAACAAGA |
| | | TTACCAATCTTCAATGGAAAAACTAGGTGTGCTCG |
| | | AAACATGGGCGACGGGGAAAAAAAGTGAAAAAAAA |
| | | GAAAGAGTCATCCGAGAAATTCCTCGTACTTGATC |
| | | AAACACCCGAGATGTCTTTCGAACAGCCAATCTAC |
| | | AATGATTTGGATTACAAAGGGTTTGAGCTGGGGCA |
| | | GGACTCGACAATTGATTTGTCATTGTTCACCAACA |
| | | ACCAATTTTTTGATCTAGACGTTTTTGCTGACGGA |
| | | GTAACCGAACTGAAGCCTGAAGTCGTTGATCCATC |
| | | ACCACAGAATGACATTTCAGTTTCCCAAACGCCTA |
| | | TTCTTTCCGTTGAAAGCTCTCCGGACAACAAGGTG |
| | | CAGAAGCCTCTAGATGATAAGCGAAGGAGAAACAC |
| | | GGCGGCTTCTGCCCGTTTCAGAATGAAGAAGAAGC |
| | | AGAAAGGAAAAGAGATGGAAGAGAAAGCCAAGCAG |
| | | CTGACGGAGACCGTTGAGCGTCTCAACCAAAGGAT |
| | | CAGGACTCTAGAGATGGAGAATAAATGTTTGAAGA |
| | | ACCTTATGTCACAAAGAGGGGCCATTGAAGACACC |
| | | AAAGACTCATCTGCCGACCCTATTTCCAAGATTGC |
| | | CGGCTCTACATCCAATTACGAACTATTGAAACTAT |
| | | TGAAGAGCAATAGCAATGACGACGGTTTTACCATG |
| | | ACGCATCTATAGTAGCATGTATCTCACTGATTAGG |
| | | GAGGGGAAGGTTTTCTGTATATTAAAAGACAAAAA |
| | | TAATAAACTAGAATTATTCATAAAGTCTCGTCTAG |
| | | AACTGTTTTGGCTCGGGAAATGTAAGAAGCGGAGT |
| | | CTTCTGTAGGATGGTCTAATTGCCATACTAGCAAC |
| | | TTGTCCATCAAAGGCTTCATCCATGGGCCGGGTTT |
| | | CTTGCCTAGTTCTTTGCAAAGTGTTTTGCCGTCCA |
| | | CGAGAGGTCTTAAAGAGTGAACCTGGGACAGATCC |
| | | TGATTTTTGATGTGTTGATATGTGGAATGATACTT |
| | | TTCAATGGCGTTACTGTCAGCTCCCTCAAAAATGC |
| | | TGAGCAAAA |
| 28 | MET28 | MSFEQPIYNDLDYKGFELGQDSTIDLSLFTNNQFF |
| | protein | DLDVFADGVTELKPEVVDPSPQNDISVSQTPILSV |
| | | ESSPDNKVQKPLDDKRRRNTAASARFRMKKKQKGK |
| | | EMEEKAKQLTETVERLNQRIRTLEMENKCLKNLMS |
| | | QRGAIEDTKDSSADPISKIAGSTSNYELLKLLKSN |
| | | SNDDGFTMTHL |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. A plasmid vector that is capable of integrating into a Pichia pastoris locus selected from the group consisting of MET1, MET3, MET4, MET6, MET7, MET8, MET10, MET14, MET16, MET17, MET19, MET22, MET27, and MET28.

2. The plasmid vector of claim 1 comprising a nucleotide sequence with at least 95% identity to a nucleotide sequence comprising at least 25, 50, 75, 100, 125, 150, 175, or 200 contiguous nucleotides of SEQ ID NO:1, 3, 5, 7, 9, 11, 13, 15, 17, 19, 21, 23, 25, or 27.

3. The plasmid vector of claim 1, wherein the plasmid vector further includes a nucleic acid molecule encoding a heterologous peptide, protein, or functional nucleic acid molecule of interest.

4. A method for producing a recombinant Pichia pastoris auxotrophic for methionine, comprising:
transforming a Pichia pastoris host cell with the plasmid vector capable of integrating into the MET1, MET3, MET4, MET6, MET7, MET8, MET10, MET14, MET16, MET17, MET19, MET22, MET27, or MET28 locus, wherein the plasmid vector integrates into the locus to disrupt or delete the locus to produce the recombinant Pichia pastoris auxotrophic for methionine.

5. A recombinant Pichia pastoris produced by the method of claim 4.

6. A nucleic acid molecule comprising a nucleotide sequence with at least 95% identity t to a nucleotide sequence comprising at least 25, 50, 75, 100, 125, 150, 175, or 200 contiguous nucleotides of SEQ ID NO:1, 3, 5, 7, 9, 11, 13, 15, 17, 19, 21, 23, 25, or 27.

7. A plasmid vector comprising a nucleic acid sequence encoding a Pichia pastoris enzyme selected from the group consisting of Lys1p, Lys2p, Lys4p, Lys5p, and Lys9p.

8. The plasmid vector of claim 5 comprising a nucleotide sequence with at least 95% identity to a nucleotide sequence comprising at least 25, 50, 75, 100, 125, 150, 175, or 200 contiguous nucleotides of SEQ ID NO:1, 3, 5, 7, 9, 11, 13, 15, 17, 19, 21, 23, 25, or 27.

9. A method for rendering a recombinant Pichia pastoris that is auxotrophic for methionine into a recombinant Pichia pastoris prototrophic for methionine comprising:
(a) providing a recombinant met1, met3, met4, met6, met7, met8, met10, met14, met16, met17, met19, met22, met27, or met28 Pichia pastoris host cell auxotrophic for methionine; and
(b) transforming the recombinant Pichia pastoris with a plasmid vector encoding the enzyme that complements the auxotrophy to render the recombinant Pichia pastoris auxotrophic for methionine into a Pichia pastoris prototrophic for methionine.

10. The method of claim 9, wherein the host cell auxotrophic for methionine has a deletion or disruption of the MET1, MET3, MET4, MET6, MET7, MET8, MET10, MET14, MET16, MET17, MET19, MET22, MET27, or MET28 locus.

11. The method of claim 9, wherein the plasmid vector encoding the enzyme that complements the auxotrophy integrates into a location in the genome of the host cell.

12. The method of claim 9, wherein the location is not the MET1, MET3, MET4, MET6, MET7, MET8, MET10, MET14, MET16, MET17, MET19, MET22, MET27, or MET28 locus.


SEQUENCE LISTING (28 sequences)

SEQ ID NO: 1 - DNA, 2677 bp, Pichia pastoris
aatgataccgttcaagacaagctcgttgtctttttcagctcccaagaatgttttccacagggcaaatagctgagatacctcatcatctgcgtcaacctcctcgttcagctctacagtaagttcagaagcatttgcactagagccagactcagcaacgccatcttcatctgtcttttgcttcttcttctgtgcggactttcccaatccaagcggtcttttgggtggagccattagctgataatcatacaggaaagtaagaaaaaagaaagaaagttttgacttcagcctcgcctcggctcgactgtctcccctattcttgcatctgcttacataagttgaaaagtcgcttggtaacatacggaggagatatcaaggttctcatctatctcgcatgccatacaaatcacgtgcgattgcatgaagcgatgagtaggcctttgaaaaaaaaaaaacagtttcataagattaggtcttcgttatcctctatccatacccccgacgatggccaaactattactcgcagataactgccaaggtcaaatccatcttgtggtgggcctagagcacctgaatttgtgtgtttcaagggtgaagactattctggaggctggagccacaccggttctagtttccccacaaaagtccacgatgctggattctcttcaagatctagccacccagggcacattgaaggtcgtagatcagaccttcagtatctcacagttgactcaattggggcgagatgaagtagataatgtggtagacaaggtgtttgtggtcttggactcgcaatacgcccaattgaaaaaagacatctcggctcactgtagaaggctaagaattcctgtttcagtggtagattctccagaattatgcagtttcactctgttatcaacctattccaatgctgattttcagctgggagtgacaactaatggaaaaggatgtaaattagcatctcgtatcaaaagagaactagttagcactctaccttcaaatattgacaaggtttgcgaaaacattggtaacctaagacacaggattcagcaagaggatgacgatcaagtggaggagatttacaataggttacaattgctaggagaagatgaagatgatgctattcagacatccagactcaaccagttggttgaggagtttaacatgaccaaagaacagaaaaaactacaaagaacgcgctggttgtcgcagttagtagagtattaccctctaggaaaactggcagaagtttctgtggacgacttaagtgctgcatatcatgaatctagtaacaacgttgaaattgctcagaatggaactttcgaccatgcgaagaaaggttctatatcattggtaggagcaggaccaggagctgtctcactactaaccttgggagcactgtccgaaatatactctgcagatctaattcttgcggacaaactagtaccgactcaagttttggacttaattcctaggagaacggaagtttttattgctagaaagtttccaggaaatgctgaagccgcacaacaggaactattatccaagggtttagcagccttagatgctgggaagaaagtaattcgcttgaagcaaggtgacccatacatttttggaagaggtggggaggaatacctatttttcgaatctcaaggttacagaccattagttttaccaggcatcacttcagcattggcagcacctgttctgtctcaaattcctgcaacgcatcgtgatgttgcagatcaagttctaatctgcacaggaactggacgtagaggagcacttccaaatattccagaatttgtgaaatcccgtacttcagtattccttatggcattgcatcgtattgtggagcttctccctgtcctttttgagaaggggtgggatccaaaggttcctgcagcaattgttgaacgagcatcctgtccagatcaaagggttattagaactacattagaaaacgttggtcgagcagtccaagaatttggttccaggcctcctgggcttcttgtggtaggatattcatgtgggatcattgaaaagttagagaaggagtgggaagtggtggaaggttgggatgacattggaggatcgaccatactagatacagtgtccaacctttccaaatgactatgaagatagtgaactgcattttatttattgtatatgtattttagacgcattaatagagagccaaaaagttatatcacaagttgatctgtagtgtcaggttgattccatgaggatcaaagtgccatccacccatcctgggtaatcatgcaaaaaatgaaagattggacgagttgggaatcgaacccaagacctctcccatgctaagggagcgcgctaccaactacgccacacgcccattttctcttcggtgaaggctttaaaagattttgacctaatcactattctttcggttttaatactaccataaaatgacagttaactactgtgcagatagcttcatacatacttagacaccttattgataaaaaaaaatgacactaggcgccgagaaccttatttacttcctaattactatgataataagttcaatctataataacctgtgcttatgtaatcattatccgcgtgtttcctccacccataattcttcaactagttttctaaccaattgattgagtttgaccatgttctccaactcaattag

SEQ ID NO: 2 - protein, 542 aa, Pichia pastoris
MAKLLLADNCQGQIHLVVGLEHLNLCVSRVKTILEAGATPVLVSPQKSTMLDSLQDLATQGTLKVVDQTFSISQLTQLGRDEVDNVVDKVFVVLDSQYAQLKKDISAHCRRLRIPVSVVDSPELCSFTLLSTYSNADFQLGVTTNGKGCKLASRIKRELVSTLPSNIDKVCENIGNLRHRIQQEDDDQVEEIYNRLQLLGEDEDDAIQTSRLNQLVEEFNMTKEQKKLQRTRWLSQLVEYYPLGKLAEVSVDDLSAAYHESSNNVEIAQNGTFDHAKKGSISLVGAGPGAVSLLTLGALSEIYSADLILADKLVPTQVLDLIPRRTEVFIARKFPGNAEAAQQELLSKGLAALDAGKKVIRLKQGDPYIFGRGGEEYLFFESQGYRPLVLPGITSALAAPVLSQIPATHRDVADQVLICTGTGRRGALPNIPEFVKSRTSVFLMALHRIVELLPVLFEKGWDPKVPAAIVERASCPDQRVIRTTLENVGRAVQEFGSRPPGLLVVGYSCGIIEKLEKEWEVVEGWDDIGGSTILDTVSNLSK

SEQ ID NO: 3 - DNA, 2706 bp, Pichia pastoris
cgcaagataatggtggcgtttcgtcgtctccccaacttgaagagttattctgagttgcaacaagtctaagtagtaagtaattaaaccatcatgatcctatgatcgtgatcattcattaaagcacggtgtggcaattattgctagggagatcgtcactgtatggtggcagaattatctctacaagatgtctcaaagtccccacaaagcttggaccctctcatctgtaatgcattttcctgtaactccccttagccacacgtcaagggctctgaatccgttgaaaagctgtggcgtctgccacctttaacgtcttcatgagggatgtgcacgtgatattgtctttcccttctctaaagcttcgaaaaaaacgcatctcaatgcgagaagcagatcgatatatataaagaactagtccattgaaagatctctcaatttcactggaaaccaactcagaaagaaatgccttctcctcacggtggtgtgctacaagaccttattaagcgtgacgcttctatcaaggaagatttgttgaaggaagtccctcagcttcaaagtattgtgctaactggtagacaactctgtgatttagagttaatcctaaatggaggtttcagtcctttgacaggatttctgaccgagaaggattatcgctccgttgttgacgatttgagactcgccagtggtgatgtttggtctattccaatcaccctggacgtcagcaagaccgaggctagtaagttccgtgtcggcgaaagagtggtgttgagagatcttcgtaacgacaatgctctgagtattctgaccatcgaggatatatacgaacctgataagaacgttgaggctaagaaagtcttccgcggtgatccagaacacccagctgtcaagtacctctttgatgttgccggtgatgtgtatattggtggcgctttgcaagctctacaattgcctactcattacgactacaccgccctgagaaaaacgccagcccaattgaggtctgagtttgagagccgtaattgggaccgtgttgtcgctttccaaacccgtaacccaatgcacagagcacaccgtgagttgacagttcgtgccgccagagctaacttggccaatgtcctgattcatccagttgttggtctgacgaaaccaggtgacattgaccaccacactcgtgtcaaagtttaccaagagatcattaagaagtatccaaacggtatggctcagttgtccctgttgccattggctatgcgtatggctggtgaccgtgaggctgtttggcatgctatcatccgtaagaactacggtgcttcacacttcattgttggacgtgatcacgctggacccggtaagaactccgctggtgttgacttctacggaccttatgatgcacaggaattggtagagaaatacaaagatgagttggacatccaagttgttcctttccgtatggttacttatcttccagatgaggatcgttacgctccaattgacacagtcaaggagggtacccgtaccctaaacatttcgggaactgagctgcgtaaacgtctcagagatggtacccacattccagaatggttctcttacccagaagtcgttaagattttgagagaatccaatccacctcgtccaaaacaaggtttcactttgtacttgaccggattgccaaactccggagttgacgccttgtccaacgctttagttgctacattcaatcaattcgaaggcgcccgccacattactctgctagatggcaagaacgtcaacgaatccgcattgccatttgttgcccatgagttgacacgctctggggctggtgtcatcattgctgaccctaccaaggctccttccgctgctgagattgattctattcgcaaggaagtatccaaggcgggctcctttatcgtgatttcattgactactcctttgaatcaagtctctcagcatgatcgtaaaggatactactccacttctcgtaaagatgttgacaactacgttttcccagaagatgctgagatcaagatcgacttggccaaagaaggtgccatcgttggtatccaaaaggtggtcttgtatttggaagaacaggggttcttccagttctagatagtagactttataatgatagattgagattatgcgaatctttgaatcgaggggaatggtaacatctgacatcttctatctcacgtctgacacgtcttgtttctcctagcgatcgatcactcctgtcgaccctctgcccccgaaagattcggtcaaaaagcaaaggcaaactatcctcactatttacatcgcagtccatttttttattcaaacaatttgctgattaacgcaattgcaaacggaccaatcacactccggctcccagaatctaggcatcttttctacacttaaaaactgaaaaactccgttcacgtgcatggtcgtgtcccttgcaattattccgtaggtatctctccactgggaaacaaaacaatcctatccgacaaacaatcgtcagaaccattaccacccgttgaatcctctgctgttaacccctaatttcggtgctcaatagctttttcaaatactaagtgataacatactcattatttgaagtttgattttagtgagaaacgagactacccaaacatttgagcgcattcaaatttttgccatctgacaaccgagaattgagaatttgagaaccattcaacgattacgtaa

SEQ ID NO: 4 - protein, 547 aa, Pichia pastoris
MPSPHGGVLQDLIKRDASIKEDLLKEVPQLQSIVLTGRQLCDLELILNGGFSPLTGFLTEKDYRSVVDDLRLASGDVWSIPITLDVSKTEASKFRVGERVVLRDLRNDNALSILTIEDIYEPDKNVEAKKVFRGDPEHPAVKYLFDVAGDVYIGGALQALQLPTHYDYTALRKTPAQLRSEFESRNWDRVVAFQTRNPMHRAHRELTVRAARANLANVLIHPVVGLTKPGDIDHHTRVKVYQEIIKKYPNGMAQLSLLPLAMRMAGDREAVWHAIIRKNYGASHFIVGRDHAGPGKNSAGVDFYGPYDAQELVEKYKDELDIQVVPFRMVTYLPDEDRYAPIDTVKEGTRTLNISGTELRKRLRDGTHIPEWFSYPEVVKILRESNPPRPKQGFTLYLTGLPNSGVDALSNALVATFNQFEGARHITLLDGKNVNESALPFVAHELTRSGAGVIIADPTKAPSAAEIDSIRKEVSKAGSFIVISLTTPLNQVSQHDRKGYYSTSRKDVDNYVFPEDAEIKIDLAKEGAIVGIQKVVLYLEEQGFFQF

SEQ ID NO: 5 - DNA, 2714 bp, Pichia pastoris
tggtgaaccaagaggcgattccatctaccagaggctgttctggacctggcaccacaagatcaacattgttctcctgagcgaactggactagttgtgggaaattctccttggaagagccgatattgacattggtaactttgtcaagtttatgggtaccaccgtttccaggagcgacataaactttggcaaccttgggggattgaatgagtttccagaccagagcattctctctgcctccgttaccaacaaccagaatggtagacattttgcgtttaagataggatttgggtagtttaggcgatgattaattgcaaagggaaattttttttttttcatttttccttctacgaatctgggggagaaggtggtgggaggatgcaggttgtagaagggaactcctggtttcctggaaggaaggagcgtagcgcggcggggtcagaccgactgacatggctgcagcagtgcgatgcgaaaaaaaaaaatctgaataaatgacacacccaacgtcatcgtgaaaagaaaaacaaatgtattatgtaatcactgaaacgtttcttccaacgtccggttagacccgaaaactcgcagatatctgtaaacatctccaaacctcctcaaaatccagttgccgaaaaaaaaaacatgtcatgccatatcacgtgagatggcgaagccactgaaaagaattatcctgcttaggtaatgtcccccagaatctagcaaaattactattcccccatagtctagccaagacacaaagttgcttagctctcaacacttaagcaaccacgtccaggactctactcgtcacaaaggccaatagaaagcctctagaagtatctcaacatcaccttcaagtccggctcaaataggtctttttagtttattcaaagttttttttcaaaccgtttgagattttctccttccaagaactcaattccacattcaacttcccttggtctgtggcttcaactcgagattcaccagatatattaggagcagatccactacaatgtcattcagcagagagaacatggtcgaaacaaatctccttaatggaaccagccaggatcaggataatacggaaacgtcagctgctctgttggagcagttggtctatattgatcatctgaacattcccgacgtcgacccgacaaatttcgatgatcaactgtctgctgagctagcagcttttgccgacgactcatttattttccccgatgaagagaagccgaagaataacggcaatgatgagccaaatgatcctgctactgtttccacgatcggcactaacactccttcaccgttgaactttcagcgacaagaccgtggccatggaagacaaaagtctggcactgaattatcaggtcttccgaaggcggtcgttcctcctggtgctatgtcctctctggtagcagctggtctgaatcaatcccagattgataccttggccacgttggtagcgcaataccaacatttacctcaaccacagcaacaacgacaacaagcaaactacctgcaatcagtgaacccaaatcttaatgaaagaaccatcttgagcctaaacgacgtattcaactacaactctggctcgagtaatccttccaatagagatgcgaccagcactacgagccccatttcaccttacgagcaaattcatggggttcagtcaaatggtcagcagcgtcgtggtaatcagacggagtcggtttcatctctcagttttaacaattctgctagtgtagaaccatcttctgtccagcagggacttcgaaagtcatccaatgcgtcgtcggcacaggtgccagagcataaatatatggcagatgacgataagagaagaaggaacactgcagcctctgccaggttccgtataaaaaagaagatgaaagagcaagctatggagcgcaatataaaggagctgacggagaatgctgaaaagttggaactaaaaatccaaaggcttgaaatggaaaatagattattacgcaacttggttgtggaaaaaggtgcccagagggactctcaagatttggagagacttcgtcgtaaggcacagctgaaaactgataactccgagtccggggcttcgaatttggaaccagtgttgaagcaggaaccaatatgagtcttaaggcgatggggtgaaatagtcgttcgtttttgtatactaccctttgaaagggatttattgaatatttagtttaagtctgatgattagatgctcagtttgtgctactatggatccaggacgaggtagtaaggaatgctagagacttgccggtcttaggaagcccatccatgggagggagccgtctaccacatattatttctagtgtcgttcaggatcccggaagtggaacctctctgaaagaagcgaaaaaaaaactagaactatttcaacgctcgtaaattagacaatcgcttggaagagataatgcccatcagtttatcatccgttgttggcttttgtagggtccccaatggcgtcattaagggtctacctcatgagtccctcgtagcatcgacctggccctctcggcccagatgttccttgcagtgttccgacatgcttcaggttttttcgcgcgagcttgtttacacatctcctaaacaagacatatcagacagcattctcatttggttcataatatccaactcaaaccattgtttcacctccgtctatcaatcctgaccctgagtcttctggtcac

SEQ ID NO: 6 - protein, 371 aa, Pichia pastoris
MSFSRENMVETNLLNGTSQDQDNTETSAALLEQLVYIDHLNIPDVDPTNFDDQLSAELAAFADDSFIFPDEEKPKNNGNDEPNDPATVSTIGTNTPSPLNFQRQDRGHGRQKSGTELSGLPKAVVPPGAMSSLVAAGLNQSQIDTLATLVAQYQHLPQPQQQRQQANYLQSVNPNLNERTILSLNDVFNYNSGSSNPSNRDATSTTSPISPYEQIHGVQSNGQQRRGNQTESVSSLSFNNSASVEPSSVQQGLRKSSNASSAQVPEHKYMADDDKRRRNTAASARFRIKKKMKEQAMERNIKELTENAEKLELKIQRLEMENRLLRNLVVEKGAQRDSQDLERLRRKAQLKTDNSESGASNLEPVLKQEPI

SEQ ID NO: 7 - DNA, 3170 bp, Pichia pastoris
acgcatattgagacagtagcgactctgtcttgttctccaattgcaacgcttgggaccttgtttgggagtagttcgacattgggttcctctgagatgtttgacaagtgagagctaaatgataacgaaatgcctacctggcaggacgtgtactgatcaaacctcccaggttcacatcggtcacttgctcgattccagcaagctacgccctttaagttttgtccaccagctttgcgcactctcttgcctctttcgaaccccgagcgcgcttcagatgcagatcaaagcacgagatgccacgtgacagtccatgtattctttcgtttatcttcgtatagacaataatatttcattgactctgtcaatggtcgatgttcacgtgcaaaaattttcaattcgtttgttgggcgacacctccactacgtatataaaaggatccgaccgcccacttgtccttgcttcctgtaattgtttcccaaacaactagtagttcaattattactaaaatggttcaatcatctgtcttaggtttcccacgtatcggtgcctttagagaattaaagaagaccaccgaggcctactggtctggtaaggtcggaaaagacgagcttttcaaagtcggaaaggagatcagagagaacaactggaagctgcaaaaggctgctggtgtcgatgtcattgcttccaacgacttctcctactacgaccaagttcttgacctgtctcttctgtttaacgctattccagagagatacactaagtacgagttggacccaattgacaccctattcgccatgggtagaggtttacaaagaaaggccaccgactccgagaaggctgttgatgtcaccgctttggagatggttaaatggtttgattctaactaccactacgtcagacccactttctctcactccactgagttcaagctgaatggtcaaaagccagttgacgagtacttagaggccaagaaacttggaattgagactagaccagttgttgttggtccagtttcttacctgttcttgggtaaggctgacaaagactctcttgacttggagccaatctctcttttggagaagattttgcctgtctacgctgaactactggccaagctgtccgctgctggtgccacttccgtgcaaatcgatgagccaatcctggttttagatctcccagagaaggttcaagctgctttcaagactgcttatgaataccttgccaatgctaagaacattccaaagttggttgttgcctcctacttcggtgatgtcagaccaaacttggcttctatcaagggtttaccagtccacggtttccactttgactttgtcagagctccagagcaattcgacgaagttgttgccgcattgacagctgagcaagttttgtccgtcggtatcattgacggtagaaacatctggaaagctgatttctccgaggctgttgctttcgttgaaaaggctattgctgctttgggtaaggacagagttattgttgccacctcttcctctttgttgcacacaccagttgacttgaccaacgaaaagaagctggactccgagatcaagaactggttttcgtttgctacccaaaagttggatgaggttgttgtcgtcgccaaggctgtatctggtgaggatgtcaaggaggctttgtctgtaaatgccgctgccatcaagtctagaaaggactctgctatcactaacgatgctgatgttcaaaagaaggttgactccatcaatgagaagttatcttccagagctgctgctttccctgaaagattggctgctcaaaagggcaagttcaacttgcctttgttcccaaccaccaccattggttctttcccacagactaaggatatcagaatcaacagaaacaagttcaccaagggtgaaatcactgctgagcaatatgacactttcatcaaatctgagattgagaaagtcgtcagattccaggaggagattggtttggatgttcttgtccacggtgaaccagagagaaacgatatggttcaatactttggtgagcagctgaagggttttgccttcaccaccaatggttgggtccaatcttacggttctcgttacgttagaccacctgtggttgtcggtgacgtttctagacctcatgccatgtctgtcaaggagtctgtttacgctcagtccatcactaagaagcctatgaagggtatgttgactggtcctatcaccgtcttgagatggtctttcccaagaaacgacgtttcccaaaaggttcaagctctgcaattgggtcttgctctgagagatgaagttaacgacttagaggccgcaagtgtcgaagttattcaagttgacgagccagctattagagaaggtttgccattgagaagcggtcaagaaagatctgactacttgaaatacgctgctgaatctttcagaattgctacttccggtgtcaagaacactactcagatccactctcacttctgttactctgatttggatcctaaccatatcaaggctttggacgctgacgttgtctctattgagttctctaagaaagatgatcctaactacattcaagagttctctaactaccctaaccacatcggattgggtttgtttgacatccactctccaagaattccttccaaggaggagttcattgccagaattggtgagattcttaaggtgtacccagctgacaagttctgggtcaaccctgactgtggtttgaagaccagaggctgggaggaggtcagagcctctttgactaatatggttgaagctgctaagacctaccgtgaaaagtacgctcagaattaagcctgaataaattctttgcgtattgattacatgctgcatttattcaacattaatgttttgcatataatgatcatatttgaatcattatcattttgttcaattacttctttctagacgatcgtttgtattatgtgttataggggggatttcaacatcggttaattaaagtttattactacttttgtgatctgtaggaaaattagtcttgtagtgtagagtggacaggcagacgcagggaagactcacttcaccagttcgagagcaggaacggacccacgattcctcccagcaaaaccgtgggcccttcagatatcacttcgctagatttctagtggcaactcctttttgaaccctattaaa

SEQ ID NO: 8 - protein, 768 aa, Pichia pastoris
MVQSSVLGFPRIGAFRELKKTTEAYWSGKVGKDELFKVGKEIRENNWKLQKAAGVDVIASNDFSYYDQVLDLSLLFNAIPERYTKYELDPIDTLFAMGRGLQRKATDSEKAVDVTALEMVKWFDSNYHYVRPTFSHSTEFKLNGQKPVDEYLEAKKLGIETRPVVVGPVSYLFLGKADKDSLDLEPISLLEKILPVYAELLAKLSAAGATSVQIDEPILVLDLPEKVQAAFKTAYEYLANAKNIPKLVVASYFGDVRPNLASIKGLPVHGFHFDFVRAPEQFDEVVAALTAEQVLSVGIIDGRNIWKADFSEAVAFVEKAIAALGKDRVIVATSSSLLHTPVDLTNEKKLDSEIKNWFSFATQKLDEVVVVAKAVSGEDVKEALSVNAAAIKSRKDSAITNDADVQKKVDSINEKLSSRAAAFPERLAAQKGKFNLPLFPTTTIGSFPQTKDIRINRNKFTKGEITAEQYDTFIKSEIEKVVRFQEEIGLDVLVHGEPERNDMVQYFGEQLKGFAFTTNGWVQSYGSRYVRPPVVVGDVSRPHAMSVKESVYAQSITKKPMKGMLTGPITVLRWSFPRNDVSQKVQALQLGLALRDEVNDLEAASVEVIQVDEPAIREGLPLRSGQERSDYLKYAAESFRIATSGVKNTTQIHSHFCYSDLDPNHIKALDADVVSIEFSKKDDPNYIQEFSNYPNHIGLGLFDIHSPRIPSKEEFIARIGEILKVYPADKFWVNPDCGLKTRGWEEVRASLTNMVEAAKTYREKYAQN

SEQ ID NO: 9 - DNA, 2368 bp, Pichia pastoris
tgacttcatggagaacatttctttggccggtaaaaccaacttcttcgaaaagagagtttctgattaccaaaaggcaggtgtcatggcttctacagacaaaacttctaatgatgatgcctttgcctttgatgaggatttctagatcttttttggtcaataataggggggttttttacaaaggttagcggttagagacttaacgtcatattacgttataatgtatattaaatttagttatgataatttttcgttatctggtaactttaggcttggtttctgttattctttttttttcttttttatttatccctcacggacggatagatgcccgaattaaacaaggaattcttcatagcgatcccctttaagcagttacttcccagcgccctcctagagtcttttcttggttgcctgcacactacccaaaaactttaaaaacgtcaggcctgccagagattttcctctctttgttcgatccaaccagtatgggacagccagatatgccattacatcgttcgtataaagatgctataagggccttgaactcccttcagtccaactacgccacaattgaggctattcgaaagtctggtaacaacagaagtgctaataacatccctgaaatggtggaatggaccagaaggataggttactctccaaccgaattcaacaggttgaacatcattcatgtgacggggactaaaggtaagggttccacatgtgcatttgtgcagtcaattttgaagagatacaagaacaaagacttcgccacagcgtccagaaactcaagtagctccacccttgcaagttcaagatccaatgaacttgaaaaaccccacataaccaaggttggattatattcctctccacacttgaagtctgtgcgggaacgtatcagaatcaatgggaagcctctaactgaggaccttttcaccaaatacttctttgaagtatgggacagacttgaaaactctgaatctaacccttctacgttccctcagttgagcccaggtttgaaacctgcctacttcaaatatttaaccctactgtctttccatgtattcatgagtgaaaacgtcgattctgccatctacgaagttggagttggtggagagttcgattccacgaacataatagaaaaacccacagttactggagtttctgctcttggcattgatcacactttcatgctgggaaataccctcacagatattgcctggaacaaatctggtatattcaaagaaggagttccagctgtttcagtaccacaaccagaggaaggtatgaatgaactcgtcagaagagctgaagagagaaaggtaaagttcttcaaagtcgttcctgacagggatctcagtgatatcaaactgggactcgcaggtgctttccagaaagagaatgcgaacttggccatagagcttgccgcaattcacctacagaaattgggattcaaagttgatgtaaaggatgaccttccagatgaatttgtggagggtttatctagcgcaacgtggcctggtagatgtcagattatagaagaacccgagaaccaaattacttggtatttggatggtgcccataccaaggaaagtatcgaggcttcttcccagtggttcactgaaaagcaaaccaagtctgatcaaactgtacttttgtttaatcagcaaactagagatggtgaagcactgattaaacagttgcatggcgtagtgtacccgaaattaaagttcaaccatgttatcttcactactaacttaacgtggtcagacggatactctgatgacctcgtgtctttgaacatctccaaagaggaaattgataatatggatgttcagaaggcacttgctgaaacttggaacagtctcgataaagcaagtcgtaaacatatttttcacgatattgaaacatccattaactttattcgttcgctcgaaggttctgtggacgtttttgttaccggatctttacacttggtgggaggattcctggttgttttggatagaaaagatttgcctaattaatttattgactgcttattaaaaaaatccccttttcttcctggacccatctaatctctaatgttgcaatagatccggaatgtccagcaattcctcttcttcgtcaatgtccaggactttgctaacacctgccttgtttcggaaaagctctactgctcctgcatacaacattttgccctcttgagtagacgtttggggcctgaagtacaccaggaccaggggtgaagattttcttccatcttgcagtgttattggatatgacaacagtataaatcttggcgaactatcaggaacttcatctaccaagtcctctaaagaggtaatgacatcagtttcagccttgatttcgt

SEQ ID NO: 10 - protein, 511 aa, Pichia pastoris
MGQPDMPLHRSYKDAIRALNSLQSNYATIEAIRKSGNNRSANNIPEMVEWTRRIGYSPTEFNRLNIIHVTGTKGKGSTCAFVQSILKRYKNKDFATASRNSSSSTLASSRSNELEKPHITKVGLYSSPHLKSVRERIRINGKPLTEDLFTKYFFEVWDRLENSESNPSTFPQLSPGLKPAYFKYLTLLSFHVFMSENVDSAIYEVGVGGEFDSTNIIEKPTVTGVSALGIDHTFMLGNTLTDIAWNKSGIFKEGVPAVSVPQPEEGMNELVRRAEERKVKFFKVVPDRDLSDIKLGLAGAFQKENANLAIELAAIHLQKLGFKVDVKDDLPDEFVEGLSSATWPGRCQIIEEPENQITWYLDGAHTKESIEASSQWFTEKQTKSDQTVLLFNQQTRDGEALIKQLHGVVYPKLKFNHVIFTTNLTWSDGYSDDLVSLNISKEEIDNMDVQKALAETWNSLDKASRKHIFHDIETSINFIRSLEGSVDVFVTGSLHLVGGFLVVLDRKDLPN

SEQ ID NO: 11 - DNA, 2136 bp, Pichia pastoris
aaggaagggaagtagataataacaaatagcaatcagagcttagccttgggtggcaaacttgctttcagtggcaaaacagtttttttcctggaagagtcttcttctttgccgactatcattgcttgccattgcacatccatattgtagttcttcgaccttggactatggtgagaagaggagttaaaagtagcaacatccaagttttatcgcgattagttatccgggtaacccataaggcagcttgccacgtcgccatcaaattggatgaattggggctgtactgcgggcttagaccagatggttgagcgacatgggagaacacggataagtccattccaatgcgtattattggaagaatactttacccagacagacattactaggagaatacgtagctaatctaggacaagtgattggtaagcagagaaaaaaacaatcaatcgcgttctgatatttaccatgtcacgaattggaaggcaaaatatcgttacccggataacagctgagcatcactcacaacacttcgtgtgttgcaagagtataattagtccaaaacgagtaactacacgtaagaacggatgtatttgagtgatacatactaagtacaacctccacgttaattactcaaattatattgagtgatggacccccgaattttccgcagtgattgaaatgtttcaactgaaagtccgcattgactaacaactctgggtgtgaagtgatcaccgataaagttacatcccttccttaccgacagctcgtttctcacactccgtctgtttcttgcaatccaagctgaattcttcgaccaatttagggatttcagaggtgtcaacttatatattcattctctttttcaccatcagcgtgctccatcttatcatcacatttaactgcgcgaaagattccattaaccccaggcggattaaaatgccattaacaccagttttggaactaatccatcatgtcaatcgaaatcccagagcccaacggttctttgatgttggcttggcaagtaagaaatcgtcatgtacttcttgtgggtggaggagcagttgccctttctcgaattgaactacttcttcaagccgatgcaaaagttacagtggttgctcccaagatagatcctaccattgaacagtatgaaaaattggggttattatacaaagttcatagaagaaagttcctcaaagatgatttgaaaatgtatgaaggtgaagcgtccagaaagctggaccaattttctggtgtagaccattttgggcccgaagagatggagcaaatagaacaggcagttaagcaggaacaatttgcattggttctaaccgcaatagatgataaaaatctttccaagcaaatatactattggtgtaaagctgggcgaatgcaagtaaacatcgccgacaaacccaaacaatgtgatttctactttgggtcagtagtaagacaggggagtatacaaattatgattagttcaaacggaaagtctccaagattgtgtcataaacttaagcacgataagctggaacctctacttgccagcttggatgcaaaaactgcagtggacaatttggggaaaatgcgtggagaattaaggcatagggtagctccaggagaggatactcccaccatcaaagaacgaatggcttggaacactcaggtgactgacctgtttacaattgaagaatggggccaatttgacgacacagcactgaataggcttctgagtttttaccccaaagtacctcaacgtcaggacataatagtcgttccgctagagaacttttaggttacgtagtaatacatgtgataacagcatctcggtcattgatagattcaaggagatacggtaggagaagccagttctggagaattagcacctgataaattcgtgttcggggaactaggaggagctggttccttggctgataatattggactagttactgtttcttcaaagtcttccaaagacttcgaaggggagctagtcgtagcagaagaagacgctggtacttccttagatgtggcccccatcgaaccgttaccactgatgttgggggctccaatagaacttcccactggactttgaaccatataggggcccgaatactgtcccggatccatctcactataaac

SEQ ID NO: 12 - protein, 274 aa, Pichia pastoris
MSIEIPEPNGSLMLAWQVRNRHVLLVGGGAVALSRIELLLQADAKVTVVAPKIDPTIEQYEKLGLLYKVHRRKFLKDDLKMYEGEASRKLDQFSGVDHFGPEEMEQIEQAVKQEQFALVLTAIDDKNLSKQIYYWCKAGRMQVNIADKPKQCDFYFGSVVRQGSIQIMISSNGKSPRLCHKLKHDKLEPLLASLDAKTAVDNLGKMRGELRHRVAPGEDTPTIKERMAWNTQVTDLFTIEEWGQFDDTALNRLLSFYPKVPQRQDIIVVPLENF

SEQ ID NO: 13 - DNA, 4031 bp, Pichia pastoris
acatttcccaaatggggtagaaagagcttagcttcggtcgttacttcgttggacgctgacggtattgaccttttagagcgcttgcttgtctacgacccggccggccgaatctccgccaagcgtgctcttcagcactcctacttctttgatgatgcaatcactgctccgcttaccgatgctgatcacgagctacaccaatccaacatgcaagtggacacttcagcagtgtatacttgaattgttatgccaactacaagaaagaaaaaataaagttacgtaagttacccgtgatattatatatagtttcatattttataaaacagctataattataattatactccttgtcgcttctctcacatcatggcacgtgagcatgtatatcttgcaaacaccgtagacgatagagatgccacacttttcaggtctggttatcctatttttttttttaaataggaagatcttagcccaagaggattcttctatattcgttcaccggagatgccttccatttcacagcgtggttcacgtaacaattcgtttagttcggaaactacggttccatcgctcgctgaggcctctgctgtctcgccctttggtctccccactgacccagaatcgctgtacggaacgaccctgacatcggcccacactgtgatcactactgtgccttattatttgtcagatagattgtttagttatgcagctcctggtgcggatggtgccttagatgctgctgctcatctgtggaggacatatttaagacctaacgctcaaggaaatgtgcctcatttaaccagatttgatatcagatctggtgcttccaatgccattttgggttatctgtcagggctagagccttccgctgtggtgcctgttttagttcctggcgctgctttgacttatatgcgccctgttctggctgagcgtagggactcacctgtaccagtcgctttcaatgtttctgcattggattatgattttgaaacctctaccctggtgtccaactatgttgaaccattgaatgctgcccgttatttgggttactctgtgttcactccattgagcaaaaacgaggctcaaagcatcgccattttaactcatgcgctggccaacattgagccaaccctcaatttgtacgatggcccttcttacctcaaacaatctggaaaaatcgaaggcatattaactggtgaaaagctgttccagctttaccagaaactgctagctgagatcccttcttggtcgaaaatagagtcctacaagagacctgctgctgctttagcctccttgagcaaactcaccggttctagactgaaatctttcgaatacgccggccacaattcaccttcgaccgtttttgttatccatggatcagtagaatctgaacttttgttgcacactgtagaacgctttgctgagaaagacgtccaaattggcgctattgcagttagagttccgctccccttcaatattgacgagtttgcttcttcttttccatcttctaccagaagaattgtcgtcattggccaggttcaaagctcttcttcttcttctttaaagaaagatgtcgctgcctctttgttctggaaactcggtgcttctgctccagctgtcgcagagtttgtctatgagccaagcttcaattggagtagcgattccttggagtcgattattgcctcttatgaagtccttccaaaatcaacctcagccaccaaaggagactacattttctggaccgctgacaatggtcgttttgcggaagttgcttccaagattgcctattccttttcacttagggatgacaacaagctaagttacagagcaaaatttgacaatatcaatggtgcgggcgtactgcaggctcaactaagaactaattctcttgttgccaccgatattgatgcggcagacattgtcttcgtagagggtttcaagttgttgcaagccttcgatgtggtttcaaccgccaaagaaggtgctacgttaattattgcatcttcagactcaattgaagatttggacaaggttgtagagtcatttcccactactttcaaacgtgatgctgctacaaagaatttgaagattcttctcatcgacttggcatctgttggtgagcaggaaggtcttggtgctagaacgggaccaattgcttgccaggctattttttatagggttgctcaacctgagttggctgaccagctgactcgttacttgtgggaaggagcagcctctgagactgaattattggcttcagttgttgctgaagttatttccaaagttgaagaagttggtatcaaggaactttccgtcgataaagaatgggcctctcttccaacaggggaagaagaagaagtcattttaccccctagaccgcttgaaacttcatttgagcccaatcttagggaatctgcaattgtccctcctccagccatcagttccaagctcgaactctcaaagaaactcgttttcaaggagagttatggtttgactaacagcctaagacctgacttacccgttaggaattttatcgtcaaagtcaaggaaaacagacgtctgacccccgacgattactcacgtaatattttccatattgagttcgatgtctctggtaccggattgacttatgacattggagaagcgcttggaattcatggtcgtaacgaccctgcactggtcgaagagttcatccaatggtatggtctcaatggtgaagaccttatcgatgttccttctagagatgatcctaacacattagaaacccggaccatcttccagagtttggtggaaaacattgatttgtttggaaaaccacctaaacgtttctacgaggcattggctccattcgctcttgacagcagtgaaaaagctaaattggagaaattggcttctcctgaaggagctccgctgcttaaggcttatcaagaggacgaattttactcttttgcggacattttggaactgttcccatctgccaaaccaactgccagcgatttggttcagattgtctctccgctgaagagacgtgaatactccattgcttcctctcagaagatgcatcctaatgaggtccatctgctcattgttgttgtcgattggattgacaaaagaggtcgtcaaagatttggacagtgctcccattacctttctgaacttagtgttgggtctgaactggttgtcagtgttaaaccttcggtcatgaagctgccaccattgtctacccagcctattgttatggctggtctgggtacaggattagccccattcaaggctttcgtcgaagagaaaatctggcagaagcaacaaggaatggagattggtgaagtttatctgtatttgggtgctcgtcaccgtaaagaggaatacctgtatggagaattgtgggaagcttacatggacgccggaattgtcacacatgtaggagctgctttctccagagaccagcctcacaagatttacattcaagatcgtattagagagaacttgaaagagttgacctctgccatcgctgacaagaatggttctttctacctatgtggtccaacttggccagttccggacattacggcctgtttgcaagatatcatcgaaagtgatgctgctagacgtggagtcaaggttgacgctgaccatgagattgaggagatgaaggaatccggtcgttacatcttagaggtttattagagaattatgtaatctcaagcattaatttcagtagatccccgcggccttttccgcggcaaactgtatattccccacccatcgtgcgataacagagcgataagcacaactgctagtatttataagtgatagctttcccatggtctttagtctttgacatgaacttgtgatgctgtctggatgtgtgatttcggagattcaccaacaggaatacgctaataatgagtccgagatctacttggataacgcaggaatgcccatgtttgccaaatcagtgctggctgaatcaatgcaaatgatgatgttgggtccttggggcaatccacattcacagtctttggcttctcaga

SEQ ID NO: 14 - protein, 1060 aa, Pichia pastoris
MPSISQRGSRNNSFSSETTVPSLAEASAVSPFGLPTDPESLYGTTLTSAHTVITTVPYYLSDRLFSYAAPGADGALDAAAHLWRTYLRPNAQGNVPHLTRFDIRSGASNAILGYLSGLEPSAVVPVLVPGAALTYMRPVLAERRDSPVPVAFNVSALDYDFETSTLVSNYVEPLNAARYLGYSVFTPLSKNEAQSIAILTHALANIEPTLNLYDGPSYLKQSGKIEGILTGEKLFQLYQKLLAEIPSWSKIESYKRPAAALASLSKLTGSRLKSFEYAGHNSPSTVFVIHGSVESELLLHTVERFAEKDVQIGAIAVRVPLPFNIDEFASSFPSSTRRIVVIGQVQSSSSSSLKKDVAASLFWKLGASAPAVAEFVYEPSFNWSSDSLESIIASYEVLPKSTSATKGDYIFWTADNGRFAEVASKIAYSFSLRDDNKLSYRAKFDNINGAGVLQAQLRTNSLVATDIDAADIVFVEGFKLLQAFDVVSTAKEGATLIIASSDSIEDLDKVVESFPTTFKRDAATKNLKILLIDLASVGEQEGLGARTGPIACQAIFYRVAQPELADQLTRYLWEGAASETELLASVVAEVISKVEEVGIKELSVDKEWASLPTGEEEEVILPPRPLETSFEPNLRESAIVPPPAISSKLELSKKLVFKESYGLTNSLRPDLPVRNFIVKVKENRRLTPDDYSRNIFHIEFDVSGTGLTYDIGEALGIHGRNDPALVEEFIQWYGLNGEDLIDVPSRDDPNTLETRTIFQSLVENIDLFGKPPKRFYEALAPFALDSSEKAKLEKLASPEGAPLLKAYQEDEFYSFADILELFPSAKPTASDLVQIVSPLKRREYSIASSQKMHPNEVHLLIVVVDWIDKRGRQRFGQCSHYLSELSVGSELVVSVKPSVMKLPPLSTQPIVMAGLGTGLAPFKAFVEEKIWQKQQGMEIGEVYLYLGARHRKEEYLYGELWEAYMDAGIVTHVGAAFSRDQPHKIYIQDRIRENLKELTSAIADKNGSFYLCGPTWPVPDITACLQDIIESDAARRGVKVDADHEIEEMKESGRYILEVY

SEQ ID NO: 15 - DNA, 1448 bp, Pichia pastoris
tcgctatattggagaagtcagcaaggaaaacgatccaacaagccacatctctcaaacgctattgttgacagaatctgtagtgatggcacatttgtacaacaatgaccgagagtttgcatatctactgaacgatggtgtcattactaataaagttatagagggagatacctccattaaccgtttaaaactgcttttcaagaaatacggacaggcaatcagcgatgaaaaagacaccgaaacttccaaagaacaattaaagatccaacttctagacgcaatagagtcgctttaagctggaccctgactaccgcacctcacttcccaagaggatgattatcggggactggaacctgtctcactatggatacctcactccgcaaagtatcacgtatgagcacgtgactacatctatttttcaatattcgggggactgtctacaatgtatattgtacctataattcccactgaataatcgacaattcccacggagcaaaagaaagatggctactaatatcacatggcatgaaaatctcactcacgatgagcgcaaggaattgactaaacaaggcggtgtcactgtctggcttaccggactcagtgccagtggaaaaagcactatcggttgtgccttagaacagagcctgctacagagaggaaacaatgcatacagactggacggtgacaacatccgcttcgggttgaacaaggaccttggattcagcgaggatgatcgtaacgaaaacatcagaagaatcagtgaggtttccaagctgtttgcagactcttgttctgttgctattacttcattcatttcaccttacagggaagagagaagaaaagccagggaactgcacaacaaagatggattgccattcgtggaagtatatgttgacgttcctattgagatcgctgaacaaagagaccccaagggattgtacaagaaggccagagagggaatcatcaaggaattcaccggtatttctgctccttacgaagcacctgagaaccccgagctgcacgtccacacagacaagcaaactgttgaggagagtgctaaaatcattattgattatttattggagaagaaactaatcaaatagagtttgtagaataagatgatttttaagtttgtatttctagttcgtgctgatcttcttctccaatttcttccgttgagcgaccagcattttgacagcagttaaccatcggattaagtcttcttcatttggggcgcaaaacttgattcttttttccctagttatcagcaaaaaacaccatttcctgatcttgctcaatggctctaattcggttatatcaattatgtcattcagattgaaaacttgaaatggtttttcctccttggacttgaacattgacagcttcttgttagtcaagaccagcttgacagttttccattggttgtaagctttttgtttttccaatgttcc

SEQ ID NO: 16 - protein, 199 aa, Pichia pastoris
MATNITWHENLTHDERKELTKQGGVTVWLTGLSASGKSTIGCALEQSLLQRGNNAYRLDGDNIRFGLNKDLGFSEDDRNENIRRISEVSKLFADSCSVAITSFISPYREERRKARELHNKDGLPFVEVYVDVPIEIAEQRDPKGLYKKAREGIIKEFTGISAPYEAPENPELHVHTDKQTVEESAKIIIDYLLEKKLIK

SEQ ID NO: 17 - DNA, 1845 bp, Pichia pastoris
caacttcctcaccacctccacaaactcacgcgtgtatatatcagggtttctaccgtcttcgatataattgactacgtccacggggatgggaatgttcaaatctgtgttgtggagcttttgcaagtgctctacaaccttgttaatgttgttggaaagacccaattgactttccgctgtaccggcgtaatcgtgcacctgaacacccaaatggatgagggtttcgatgagttgacttagttcattttcaacttgatctaatgttgtcgcaggtgcactcatacttgtcatggagaatgaaagtaagttgatagagagcagacttcgaggatgggatgaacttgattaggtaatctttgacaatgtcttagaggtaggcagaggatgctggaaaaaaaaaattgaaaacgcccaagcttccagctttgcaaggaaagaagaaaagggagttgccagcacgaaatcggcttcctccgaaaggttcacaattgcagaattgtcaccattcaaatgcctttacccttcatctgtggtacctcaggctaagaacgggtcacgtgatatttcgacactcatcgccacaatatgtactagcaagaacttttcagatttagtaatccgttcgaaacgggaaaaaatgtttttacccttctatcaactgctaatctttctaggtttatactgccagcagcccgttccagataccaacatgccattcactataggccagtcaaaaaccagtttgaacctctccaaggtccaagtggaccaccttaacctttctcttcagaatctcagtccagaagaaatcatacaatggtctatcattaccttcccacacctgtatcaaactacggcattcggattgactgggttgtgtataactgacatggttcacaaaataacagccaaaagaggcaaaaagcatgctattgacttgattttcatagacaccttacatcattttccacagactttagatctcgttgaacgagtcaaagataaataccactgcaatgttcatgtcttcaaaccacagaatgccactactgagctcgagtttggggcgcaatatggcgaaaacttatgggaaacagatgataacaagtatgactacctcgtaaaagttgaaccctcacaacgtgcctaccatgcattagacgtctgcgccgtcttcacaggaagaagacggtctcaaggtggtaaaaggggagaattgcccgtgattgaaattgatgaaatttctcaggtggtcaagattaatccgttagcatcctgggggtttgaacaagttcaaaactatatccaagctaatagcgttccatacaacgaattgctggatttgggatacaagtcagttggagattaccattccacacaacccactaaaaatggtgaagatgaaagagcaggcaggtggagaggtaaacaaaagagtgagtgtggtatccacgaagcttctagatttgcacaatatttgaaagctcagcaaaacatatgaatataattttttttttctctacactatttatcctgtaagtttctgtttccccatgtaggatctttttctccttctctgtctcccattttttttgttccctgtagtcttgccttgcctgagatgcgagctcgtccgcccatccagtcgtgtgaagggcctagcttttcaaaaagaaaatacctcccgctaaaggaggcgttgccccttctatcagtagtgtcgtaaccaattttcacaaacaataaaaaaaggacaccaacaacgaaatcaactatttacacacatccagatccgtccccctccccatccaagagttaaagacaaatatggctgttaataatccgtct

SEQ ID NO: 18 - protein, 287 aa, Pichia pastoris
MFLPFYQLLIFLGLYCQQPVPDTNMPFTIGQSKTSLNLSKVQVDHLNLSLQNLSPEEIIQWSIITFPHLYQTTAFGLTGLCITDMVHKITAKRGKKHAIDLIFIDTLHHFPQTLDLVERVKDKYHCNVHVFKPQNATTELEFGAQYGENLWETDDNKYDYLVKVEPSQRAYHALDVCAVFTGRRRSQGGKRGELPVIEIDEISQVVKINPLASWGFEQVQNYIQANSVPYNELLDLGYKSVGDYHSTQPTKNGEDERAGRWRGKQKSECGIHEASRFAQYLKAQQNI

SEQ ID NO: 19 - DNA, 2290 bp, Pichia pastoris
cccagtatgagaggaacaggagatgagctggaatttggaaacaggaacgttcaattgccaaggagaagtttgagaggagagagtggcaaagagaatggagtcacttcctatccatgcttacaacaagatctctggaatatgacatacaacatagcaacaaagagggggtgcatcaaaaaaaaattacacgttttcccaccctttccaacgaacccccacaccagtgaggtgaacagatttaacgggtctcagataaacgaaaaaatgctaacaataccatctatcgtgagggggcggcccactgccacatttccaaaagatacccccctccgcttcagattgtaattgtctgttttatagtactgcagtgaagcgccacagctccaaaacttaatttgacttctttatcaattaccgtaatattagtcgggccttgccgcatcacgtgacccgatttcactataaaactctccgttcccataaagttttaccacatcacgtgagttgtcaacattgaaacccctcgatgtaatgcttcacaggttggttatttaaatcatccaatcgccgaccaaatgaaatgatttctaacgtttccttattcacatacaaagatgccttctcacttcgacactttgcagctgcacgccggtcagaccgctgaagctccacacaatgccagagctgttcctatctacgctacctcgtcttacgttttcagagactctgagcacggtgccaagctgttcggtttggaggagccaggttacatctactctcgtttgatgaaccctactcagaacgtctttgaagagagaattgccgctttggagggtggtgccgctgctttggctgttggatccggtcaagctgctcaattcctggctattgctggtttggctcacactggtgacaacgtcatctccacctctttcttgtacggtggaacttacaaccaattcaaggtcgccttcaagagactgggaattgaatccagatttgtccatggtgatgacccagctgaattcgagagactgatcgatgataagaccaaggccatctacgttgagtccattggtaacccaaagtacaatattccagattttgaggctctcgcagagcttgcccacaagcacggtatcccattagttgttgacaacacctttggtgccggtggttactacgttagaccaatcgagcttggtgctgacatcgtcacccactccaccactaagtggatcaatggtcacggtaacaccatcggtggtgttgtcgttgactctggtaagttcccatggaaagactacccagagaagtaccctcaattctccaagccatctgagggttaccacggtttgatcttgaatgacgcctttggaccagctgccttcattggtcacttgagaactgaactgctaagagatttgggtcctgcttcaagtccattcggtaacttcttgaacataatcggtttggagaccttatctctgagagctgagagacacgctgagaatgctttgaagctggccaaatacttggaaacctctccatacgtcagctgggtctcttaccctggtttggagtctcacgactaccacgaggccgctaagaagtacttgaagaacggtttcggtgctgtattgtcttttggagtcaaggatcatggcaagccagcgctcactcccttcgaagaggctggtcctaaggttgtagactccctgaaggttttctccaacttggctaacgttggtgactccaagtctttgatcattgctccttactacactactcaccaacagttgtctcacgaggagaagctggcttccggtgtcaccaaggactctatccgtgtttctgtcggaacagagttcatcgatgatcttattgcagaccttgaacaggcctttgcccttgtttacgaggaggcaaacacaaagttgtgagttagtttaacagttgtaattgatcaataatgtatgtgtagagtttagaatacgataatgtgtatatcattatgtcatttccattgatagtaactattggtaagtagcacagctatttgtatgtatataatttgagtaatcaaggttaaatgtaaaaataaatataagtgtcatcatcgttgtctttgacagtaagaactagttaatcatctccgtgtttgaagcagcatcttttaccgtagcggcatttgccgaacttggtccagttggcacaaggtttcgtcttccagttggaaggtctcttcacggacttcagttcgtgagtcccgtgagcaaattgacacttt

SEQ ID NO: 20 - protein, 442 aa, Pichia pastoris
MPSHFDTLQLHAGQTAEAPHNARAVPIYATSSYVFRDSEHGAKLFGLEEPGYIYSRLMNPTQNVFEERIAALEGGAAALAVGSGQAAQFLAIAGLAHTGDNVISTSFLYGGTYNQFKVAFKRLGIESRFVHGDDPAEFERLIDDKTKAIYVESIGNPKYNIPDFEALAELAHKHGIPLVVDNTFGAGGYYVRPIELGADIVTHSTTKWINGHGNTIGGVVVDSGKFPWKDYPEKYPQFSKPSEGYHGLILNDAFGPAAFIGHLRTELLRDLGPASSPFGNFLNIIGLETLSLRAERHAENALKLAKYLETSPYVSWVSYPGLESHDYHEAAKKYLKNGFGAVLSFGVKDHGKPALTPFEEAGPKVVDSLKVFSNLANVGDSKSLIIAPYYTTHQQLSHEEKLASGVTKDSIRVSVGTEFIDDLIADLEQAFALVYEEANTKL

SEQ ID NO: 21 - DNA, 2728 bp, Pichia pastoris
ggtgaaaaataccaagggcgatggaaatttcaaaggccgatctggggatgtgtggggtaaagactttggatggaatccaggggcaaagacaagggctagacttcactatattggtggtaaaagtgaatctactagaagtttgagtcaacgacgatatggagtaaccaagtgaagacgatatctttagttcgttatggccaccttaaaagaagcccactcagtccatgtgagttctgaaacttttaaagacagttaacccaaggttcacaattgtgtgaccttatgtcaactgtactagaaggccaaagattattggacgattgggttatctatttccttgataagcatgtgctccaatcaatacacccacctgtcaggggatacacagtgcggagctccgttttctcccagaaattcggttggagctcttttcttaaacttcgaaagtcccccgacagagaagtgccgttagccaatagtgtccctgcattctggttcctccccactgcagcgtcagctggaaagggctctattctaagctattctaaagcaatccaaaggtgggggtcggatcaatgcgcgatctttcgtcgccagtgtcggggcccggcacgggggccgtaaccggcttttctctaggttgacaccatgggatatcccctgattgggcaaatcccacataagtatggcttgcggcttactaatcgcgtaagtcgcgcattctctttttcctgatccttaatatcaatcctccggcaccatcatcgtagtttgcgagattccataaactttttggccccctaactttttttttgttgccatcctttacttccatctaaaaaaaccgacacagaatctgccaaacaatgaccgatacgaaagccgtagaatttgtgggccacacagccattgtagtctttggagcttcaggggacctggctaagaagaagactttccctgccctcttcggactttaccgtgagggatacctgtccaacaaggtgaagattattggctatgctagatcaaagctggatgacaaggagttcaaggatagaattgtgggctatttcaagacaaagaacaagggcgacgaggacaaagttcaagaattcttaaagttgtgctcatatatttcagctccttatgacaaaccagatgggtatgaaaagttgaatgaaactattaacgaattcgaaaaggaaaacaacgtcgaacagtctcacaggttgttctacttagctttgcccccttctgttttcatacctgttgctacggaggtcaagaagtatgttcatccaggttctaaagggattgctcggattatcgtggaaaaacctttcgggcacgacttgcagtcagcagaagagcttttgaatgctttgaagccgatctggaaagaagaggaattgtttagaatcgaccactatctaggtaaggagatggttaagaatttgttggccttccgttttggaaacgcattcatcaatgcttcttgggacaacagacatatcagctgtatccaaatctcgttcaaggagccttttggaacagaaggtcgtggtggctattttgactcaattggtataataagagacgtcattcagaaccacttgcttcaagtgttaaccctcttaaccatggagagacccgtctctaatgaccctgaggctgttagagatgaaaaggttcgcattctgaagtcaatttctgagctagatttgaacgacgttttggtgggtcaatacggcaaatctgaggatggaaagaagccagcttatgtggatgatgaaactgttaagccaggttctaaatgtgtcacatttgcagccattggcttgcacatcaacacagaaaggtgggaaggtgtcccaatcattttaagagctggtaaggctttgaacgaaggtaaagttgagattagagtgcaatacaaacagtctactggatttctcaatgatattcagcgaaatgaattggtcatccgtgtgcagcctaacgaagccatgtacatgaaactgaactccaaagtcccaggtgtttcccaaaagactactgtcactgagctagacctcacttacaaagaccgttacgaaaacttttacattccagaggcatatgaatcacttatcagagatgctatgaagggagatcactctaattttgtcagagatgacgagttgatacaaagttggaagattttcactcctttactgtatcacttggagggccctgatgcaccggctccagaaatctatccctacggatccagaggtccagcttcattgaccaaattcttgcaagatcatgattacttctttgaatcacgcgacaattaccaatggccagtgacaagacccgatgtgctgcacaagatgtaaattattctatagatttaggacgattacagatatcaatgatagtttagcttgtttcagtattacgtaataaatgactcagaggtatctcaggatctgtggggcaggaagtggcattgcatttgctcgctcctattagcttatcagggaagaggaaagaaaaattcttgcatataaagtgctgggccagcccacatccttagcacgttatcagcttttcacaactctactcctgattttctgatggaaaccccaagctatccactgaaagcaaaaaccaaagatgaaggggaaataattgtaagggatatcattctaactaaccacgaagagacacagggtcattcttc

SEQ ID NO: 22 - protein, 504 aa, Pichia pastoris
MTDTKAVEFVGHTAIVVFGASGDLAKKKTFPALFGLYREGYLSNKVKIIGYARSKLDDKEFKDRIVGYFKTKNKGDEDKVQEFLKLCSYISAPYDKPDGYEKLNETINEFEKENNVEQSHRLFYLALPPSVFIPVATEVKKYVHPGSKGIARIIVEKPFGHDLQSAEELLNALKPIWKEEELFRIDHYLGKEMVKNLLAFRFGNAFINASWDNRHISCIQISFKEPFGTEGRGGYFDSIGIIRDVIQNHLLQVLTLLTMERPVSNDPEAVRDEKVRILKSISELDLNDVLVGQYGKSEDGKKPAYVDDETVKPGSKCVTFAAIGLHINTERWEGVPIILRAGKALNEGKVEIRVQYKQSTGFLNDIQRNELVIRVQPNEAMYMKLNSKVPGVSQKTTVTELDLTYKDRYENFYIPEAYESLIRDAMKGDHSNFVRDDELIQSWKIFTPLLYHLEGPDAPAPEIYPYGSRGPASLTKFLQDHDYFFESRDNYQWPVTRPDVLHKM

SEQ ID NO: 23 - DNA, 2056 bp, Pichia pastoris
tgccatgggcttttgtcactgggttgtaagcctctagccattcggggtcatcttcactacctatgacgtgaaaaaagtctcctttcttgaaagtgagctcaccagggccctgggccttgtagtcatacagagatttgatgacttttttgggcgtatcgagaacctcggagtgggaggtatcgacttgtattggttcagccttggtgatcttgggacccttagaatgcttgtctttagaagatcttttgaaacttatcattggaagagattggtatgaaatgagagactttatgaatagcttgacaagagaagagggaagggagagaaaaggagtcgatcactgtgaaagtaatttcctttcaggtaattacgaatgttgagagtgagaatgacaagaatggtgctgggatgcaatattccgtacctttctgcatcaccccctctcaagtacgagttgtccacctgcaagaaaaaaaagcactgcgttcaggagaaaaaatatgttcagcagggaagttaagctagcccaattggctgtcaaaagggcatctctattgactaagaggataagtgatgagattgcagctcgcacagttggcggaatttcgaaatcggacgattctccagtcactgtgggggactttgctgctcagtctatcatcatcaacagcatcaagaaagccttccccaatgatgaggttgttggagaagaagactctgcgatgttgaagaaagacccaaagctggctgaaaaggtgttggaagagatcaagtgggttcaagagcaggacaaagccaacaatgggtcgttatctctgttgaactcggtagacgaagtttgcgatgctatcgacggcggcagctctgaaggtggccgtcaaggaagaatttgggccttggatcccattgatggtactaagggcttcctgagaggcgaccaatttgccgtttgtctggcattaatcgtggatggggttgtaaaagttggtgtaattgggtgtccaaatctaccgtttgacctacaaaataagagcaagggaaaaggaggacttttcaccgcagctgaaggcgtaggatcatactatcagaacttgtttgaagagatcttgcctctggaatcatcaaaaagaatcacaatgaacaattctctttcttttgatacctgcagagtctgtgaaggtgttgagaagggtcattcaagtcatgggttgcaaggattaataaaagaaaagctccagatcaagtccaagtccgccaacttggattctcaagccaagtactgtgctctgtcgagaggagatgctgaaatatatttgaggttgccaaaagatgtgaattaccgagagaaaatatgggatcatgctgctggcaacattctgatcaaggaaagcggaggcattgtgtctgatatttatggtaaccagttggattttggcaacggtcgggagctcaactcgcagggaataatcgcggcatcaaaaaatttacatagcgatatcatcactgcagtgaaaagtattattggagatagaggccaagatttggagaagtatatatagatatagcttgtactagaatatgatcacgaggctaaagaacaaaagtaaggagaggacagccgctttgaagggcaaaaagcgggcacaggaaggtattgaagcgcaagaacggaaagatctaccacccagtaagattacgcaaaggacgaagagctcaaataaagtcaccaagatgggaaaacagagctggtataacgatctttcaaagtacaatcacattaaaccattgacgtccaaagttagaggaatggtcagtaatatgactaattacaatcatctcttgatgagatctattgagaatcctcactatagacagaaactattagacattgaagaaaggaagctgcgcttgaatagctatccgctgcccaaggtacaaaatgaccagagcttgaaagatgccttgaaccactttagaattgatagacagggcagatcaattccgatactggatagaaatcctcatgtgtgttcttcattcaaagagaataagcatt

SEQ ID NO: 24 - protein, 352 aa, Pichia pastoris
MFSREVKLAQLAVKRASLLTKRISDEIAARTVGGISKSDDSPVTVGDFAAQSIIINSIKKAFPNDEVVGEEDSAMLKKDPKLAEKVLEEIKWVQEQDKANNGSLSLLNSVDEVCDAIDGGSSEGGRQGRIWALDPIDGTKGFLRGDQFAVCLALIVDGVVKVGVIGCPNLPFDLQNKSKGKGGLFTAAEGVGSYYQNLFEEILPLESSKRITMNNSLSFDTCRVCEGVEKGHSSHGLQGLIKEKLQIKSKSANLDSQAKYCALSRGDAEIYLRLPKDVNYREKIWDHAAGNILIKESGGIVSDIYGNQLDFGNGRELNSQGIIAASKNLHSDIITAVKSIIGDRGQDLEKYI

SEQ ID NO: 25 - DNA, 3340 bp, Pichia pastoris
attctctttggggtttgtctagcggctaatctgaacattttgtgtttgttgcaaggtaatagaactaaagagagttactattggagaggtatcgtgcaagaaaagagtagtccgggtaacaacgatcaatagtaggaggtgagaggtcacctcatagaatttcgtgtatttcctttacgctttttgccaatcttctgattggctggatcccccaaaatatgtcgcgcgcagcctctcactggagggccagtcggcccatattcacgtgacgcaccttcgaacccaaagggtaagctaactaaccaagaaaatactactttcccttttcaaataccaacacatagaaacaatggctgcagcttcattaaccagaattcaaggatctgtcaagagaagaatcttgaccgacatctcagttggcctgaccctcggtttcggctttgcttcctactggtggtggggagtccacaagccaaccgtagcccacagagagaactactacattgagttggctaagaagaagaaggccgaggaagcttaacttatttaaacctgtgacaaagatcaagagctgcacagtactttatattgtgtatttttaaagagcatattttgcatgacttttattggtgaacacggagatggactgtgtctttgatgatgctagcgtggtattgcaaggtgaaattaatggttttggagggcagattttagtttagcaaacttcttgccttgcgagtgaccgtccgctgtccaatccaaatacttgtagaattttctgacctggttctccccagtcaacctagaaatttgctgacatgagcccttcaaatgaagaacgttgatactttaaaactggtggctatgctgttattaaccctggtatattctctgatttctgagctaaaacatggaaggtggaaagtagcctttttgctcccaagagcacccaaagtgactctcgaaataattcttatccaaaagtaatttgttaacactgatgatagatctcagctcagttgattccaagccagtcgatgatctgtttgcaatctttgacgagatcaatcgaaagcttaacatacaatgcgatcatctgctgatcttggaaaaaaaactatctcagccaatcaactttttgacgccgttcagcgctcttcaaaaggtcaccagaataaccaaggtcatatggttagagaaccttaccgatgaaactttgcatgcagctctgaatgaatttaattctgttgtgttcttctgcgaggatagtttgcaaaacgttggacgggtggcaaaactgttccgatccaccattctacccatcactgagacgaattcaatgatgaacacatcactaataactctgggatccttaaaccaatcaattcgtctatatctgtcagagctatcattggagaatgacattgactactattcgtgggattctattctgttcagaatagacaaagatctactttctctaaattcttcctcagatttgaaaaagttgtaccaattgcaatctatcgaacctttgtatgccctggcaaatggtttgctgcatttggtgattcattctaacttcaagttaagattcacaaataaatttatcaagggtgccaattcagccaagttttatgatatctatcagaaattatacaccaactacactctgaataaattgagtccggaaaaaagaaaaatcctggaagatgtggacgagacattgttcatggatattcactcattctacaacaatcaatgcgacctgtttgtttttgagagaagcgttgattttataaccccgttattaacacaactcacatactgtggtttggtgcatgataactttaacgttgaatacaacaccgtcaacttgaaatctgaaacgataccactgaatgatgagctctaccaggaaatcaaagatttaaatttcactgttgtgggatctttgctcaattctaaagctaaatcgttacaagaatcatttgaagaaaggcacaaggctaaagacattgcacaaataaaggattttgtttccaacttaacgaacctcacaaaggaacaacaatcgttgaagaatcatactaacttggctgaggcagttctagcaaaagtacatgatgaaacgggcaacagtgaaaaccactcggaggacagcttgttcaatcagttcttggaactccaacaagatatcttatccaacaaactagacaataaaaccacctacaaatcaattcaaacttttttctgcaaatacaaccctcctcctttgctacctcttaggttgatgatcctctcctcaattgttaagaatgggataagggattatgaatttaatgcattgaagaaggatttcgttgattactatggtgtggactatcttcccgtaataaacacgcttgccgagctctcacttttgacaagtaagaagagccagcccttagaacaaaatcctaattcacaactcatcaaagacttccataatttgagcacttttctgaaccttttgcctggaacggaagaaacaaatcttctaaaccctaccgaattagattttgctctcccagggtttgttcctgtcattactagattaattcagtcggtttatacccgatctttcattgggccgaattccaatcctgtaattccatacattgcgggatctaacaaaaagtacaactggaagggtctcgatatcatcaacacatacttgactggtaccatgcagtccaaactgttgataccaaaatcaaaagagcaaatattcacccacagaactgcagcgcctcctcattcacgtaagggtgttctcagaaatgaggagtatattatagtagtcatgctgggaggtatatcgtacggagaattgtcaaccttaagggtcgccatatcgaagatcaacgagtctatgaacttgaacaaaaagcttcttgtgctcacaagttctgttctcaaaagtgatgatataatcaagctgactaaataatattgttgccctattaacgactgtacagttcatatctccttcgcttcgattcctatccctgactttcccttacagagatagagttagatgcctttagaatcagatactctagtattatcgcgcgcagtaagtgctcctaaattttcttttttttctggtttcaaacttagttaagaaagagtggacatgagaaaccttgtggtcctgaacaaaggagagatcgtggttgaatcacgaacctatcctgagttgagagtgctggattcagtatttgactccatttcagacacaattaccgtggcacttggtaagaatgaatctggaataattgaagttcaccagttcatg

SEQ ID NO: 26 - protein, 663 aa, Pichia pastoris
MIDLSSVDSKPVDDLFAIFDEINRKLNIQCDHLLILEKKLSQPINFLTPFSALQKVTRITKVIWLENLTDETLHAALNEFNSVVFFCEDSLQNVGRVAKLFRSTILPITETNSMMNTSLITLGSLNQSIRLYLSELSLENDIDYYSWDSILFRIDKDLLSLNSSSDLKKLYQLQSIEPLYALANGLLHLVIHSNFKLRFTNKFIKGANSAKFYDIYQKLYTNYTLNKLSPEKRKILEDVDETLFMDIHSFYNNQCDLFVFERSVDFITPLLTQLTYCGLVHDNFNVEYNTVNLKSETIPLNDELYQEIKDLNFTVVGSLLNSKAKSLQESFEERHKAKDIAQIKDFVSNLTNLTKEQQSLKNHTNLAEAVLAKVHDETGNSENHSEDSLFNQFLELQQDILSNKLDNKTTYKSIQTFFCKYNPPPLLPLRLMILSSIVKNGIRDYEFNALKKDFVDYYGVDYLPVINTLAELSLLTSKKSQPLEQNPNSQLIKDFHNLSTFLNLLPGTEETNLLNPTELDFALPGFVPVITRLIQSVYTRSFIGPNSNPVIPYIAGSNKKYNWKGLDIINTYLTGTMQSKLLIPKSKEQIFTHRTAAPPHSRKGVLRNEEYIIVVMLGGISYGELSTLRVAISKINESMNLNKKLLVLTSSVLKSDDIIKLTK

SEQ ID NO: 27 - DNA, 1409 bp, Pichia pastoris
acaaacataagaaaaaatccaagaataagagcaagaatgtcaggtttttggacgacctggaatccaacctggatcttgacaacacagacgataagaaggacaatagtgtgatgagcaaacttctcagctcaatgggctaccaggcgcaagaaccttacaaaccgctagataagggtgcaaacgccgatcttgacattgagatggacagtcatggtacctcggaaaagtagggctaagccaaccaatgaaatgtatagagtatgttgaaaaggtgttaggtgaataatattaaaagtgtactattcgactccggcgtttttccacgctttgaaattttccatagcctaccgcttacaaaagttgactctgtcaccccccaacaagattaccaatcttcaatggaaaaactaggtgtgctcgaaacatgggcgacggggaaaaaaagtgaaaaaaaagaaagagtcatccgagaaattcctcgtacttgatcaaacacccgagatgtctttcgaacagccaatctacaatgatttggattacaaagggtttgagctggggcaggactcgacaattgatttgtcattgttcaccaacaaccaattttttgatctagacgtttttgctgacggagtaaccgaactgaagcctgaagtcgttgatccatcaccacagaatgacatttcagtttcccaaacgcctattctttccgttgaaagctctccggacaacaaggtgcagaagcctctagatgataagcgaaggagaaacacggcggcttctgcccgtttcagaatgaagaagaagcagaaaggaaaagagatggaagagaaagccaagcagctgacggagaccgttgagcgtctcaaccaaaggatcaggactctagagatggagaataaatgtttgaagaaccttatgtcacaaagaggggccattgaagacaccaaagactcatctgccgaccctatttccaagattgccggctctacatccaattacgaactattgaaactattgaagagcaatagcaatgacgacggttttaccatgacgcatctatagtagcatgtatctcactgattagggaggggaaggttttctgtatattaaaagacaaaaataataaactagaattattcataaagtctcgtctagaactgttttggctcgggaaatgtaagaagcggagtcttctgtaggatggtctaattgccatactagcaacttgtccatcaaaggcttcatccatgggccgggtttcttgcctagttctttgcaaagtgttttgccgtccacgagaggtcttaaagagtgaacctgggacagatcctgatttttgatgtgttgatatgtggaatgatacttttcaatggcgttactgtcagctccctcaaaaatgctgagcaaaa

SEQ ID NO: 28 - protein, 186 aa, Pichia pastoris
MSFEQPIYNDLDYKGFELGQDSTIDLSLFTNNQFFDLDVFADGVTELKPEVVDPSPQNDISVSQTPILSVESSPDNKVQKPLDDKRRRNTAASARFRMKKKQKGKEMEEKAKQLTETVERLNQRIRTLEMENKCLKNLMSQRGAIEDTKDSSADPISKIAGSTSNYELLKLLKSNSNDDGFTMTHL